SEQUENCE LISTING (29 sequences)

SEQ ID NO: 1 - RNA, 23 nt, synthetic construct
  source 1..23: mol_type = other RNA
caccggagttaatataaaaaaca

SEQ ID NO: 2 - RNA, 23 nt, synthetic construct
  source 1..23: mol_type = other RNA
aaactgttttttatattaactcc

SEQ ID NO: 3 - RNA, 25 nt, synthetic construct
  source 1..25: mol_type = other RNA
caccgaagaattacaatgaaaaata

SEQ ID NO: 4 - RNA, 25 nt, synthetic construct
  source 1..25: mol_type = other RNA
aaactatttttcattgtaattcttc

SEQ ID NO: 5 - RNA, 24 nt, synthetic construct
  source 1..24: mol_type = other RNA
caccggcagcgcggctatcgtggc

SEQ ID NO: 6 - RNA, 24 nt, synthetic construct
  source 1..24: mol_type = other RNA
aaacgccacgatagccgcgctgcc

SEQ ID NO: 7 - RNA, 21 nt, synthetic construct
  source 1..21: mol_type = other RNA
gagggcctatttcccatgatt

SEQ ID NO: 8 - DNA, 21 bp, Mus musculus
  source 1..21: mol_type = other DNA
aagaacctgctagaccacctg

SEQ ID NO: 9 - DNA, 20 bp, Mus musculus
  source 1..20: mol_type = other DNA
tgcttcctcagtctgctcac

SEQ ID NO: 10 - DNA, 18 bp, Mus musculus
  source 1..18: mol_type = other DNA
gcagcagcccagtacctc

SEQ ID NO: 11 - DNA, 18 bp, Mus musculus
  source 1..18: mol_type = other DNA
gtcatcaggcacgaagca

SEQ ID NO: 12 - DNA, 22 bp, Mus musculus
  source 1..22: mol_type = other DNA
cctcacatcttctccatctctg

SEQ ID NO: 13 - DNA, 22 bp, Mus musculus
  source 1..22: mol_type = other DNA
ttggatgaccctcttagtgttg

SEQ ID NO: 14 - DNA, 19 bp, Mus musculus
  source 1..19: mol_type = other DNA
gctccaagcagatgcagca

SEQ ID NO: 15 - DNA, 18 bp, Mus musculus
  source 1..18: mol_type = other DNA
ccggatgtgaggcagcag

SEQ ID NO: 16 - DNA, 4334 bp, Homo sapiens
  source 1..4334: mol_type = other DNA
gctcatcgtttgttgtttagataatatcatgaactgataaatgcagttgccacgttgattccctagggcctggcttaccgactgaggtcataagatattatgccttctctttagacttggtcagtggagaggaaatgggcaaagaaccagcctatggaggtgacaaggccttagggccaaaagtcttgagggtgaaggtttagggcctgcgcagcttccctgccatgccccgcaaggtctcgcattcgcaaggcttgtgacagtgggagcctcattacggactctcctaaagtccatggtgtcctcttttcgcatttgcgccccgtgggtgatgcccgatgccgcccttcccatcgctctcttccccttcaagcgtatcgcaactgcaaaaacacccagcacagacactccattttctatcttaatgcatttaactagcacaacctacaggttgttccatcccagagactacccttttctccatagacgtgaccatcaaccaaccagcggtcagaatcagtcagcctctgtcatgttcctaggtccttggcgaactggctgggcggggtcccagcagcctaggagtacagtggagcaatgcctgacgtaagtcaacaaagatcacgtgagacgaatcagtcgcctagattggctacaactaagtggttgggagcggggaggtcgcggcggctgcgtggggttcgcccgtgacacaattacaactttgtgctggtgctggcaaagtttgtgattttaagaaattctgctgtgctctccagcactgcgagcttctgccttccctgtagtttcccagatgtgatccaggtagccgagttccgctgcccgtgcttcggtagcttaagtctttgcctcagcttttttccttgcagccgctgaggaggcgataaaattggcgtcacagtctcaagcagcgattgaaggcgtcttttcaactactcgattaaggttgggtatcgtcgtgggacttggaaatttgttgtttccatgaaatcctgcgcagtgtcgctcactaccgccgctgttgccttcggtgatgaggcaaagaaaatggcggaaggaaaagcgagccgcgagagtgaagaggagtctgttagcctgacagtcgaggaaagggaggcgcttggtggcatggacagccgtctcttcgggttcgtgaggcttcatgaagatggcgccagaacgaagaccctactaggcaaggctgttcgctgctacgaatctttaatcttaaaagctgaaggaaaagtggagtctgacttcttttgccaattaggtcacttcaacctcttgttggaagattattcaaaagcattatctgcatatcagagatattacagtttacaggctgactactggaagaatgctgcgtttttatatggccttggtttggtctacttctactacaatgcatttcattgggcaattaaagcatttcaagatgtcctttatgttgaccccagcttttgtcgagccaaggaaattcatttacgacttgggctcatgttcaaagtgaacacagactacaagtctagtttaaagcattttcagttagccttgattgactgtaatccatgtactttgtccaatgctgaaattcaatttcatattgcccatttgtatgaaacccagaggaagtatcattctgcaaaggaggcatatgaacaacttttgcagacagaaaaccttcctgcacaagtaaaagcaactgtattgcaacagttaggttggatgcatcataatatggatctagtaggagacaaagccacaaaggaaagctatgctattcagtatctccaaaagtctttggaggcagatcctaattctggccaatcgtggtattttcttggaaggtgttattcaagtattgggaaagttcaggatgcctttatatcttacaggcaatctattgataaatcagaagcaagtgcagatacatggtgttcaataggtgtgttgtatcagcagcaaaatcagcctatggatgctttacaggcatatatttgtgctgtacaattggaccatgggcatgccgcagcctggatggacctaggtactctctatgaatcctgcaatcaacctcaagatgccattaaatgctacctaaatgcagctagaagcaaacgttgtagtaatacctctacgcttgctgcaagaattaaatttctacagaatggttctgataactggaatggtggccagagtctttcacatcatccagtacagcaagtttattcgttgtgtttgacaccacagaaattacagcacttggaacaactgcgagcaaatagagataatttaaatccagcacagaagcatcagctggaacagttagaaagtcagtttgtcttaatgcagcaaatgagacacaaagaagttgctcaggtacgaactactggaattcataacggggccataactgattcatcactgcctacaaactctgtctctaatcgacaaccacatggtgctctgaccagagtatctagcgtctctcagcctggagttcgccctgcttgtgttgaaaaacttttgtccagtggagctttttctgcaggctgtattccttgtggcacatcaaaaattctaggaagtacagacactatcttgctaggcagtaattgtatagcaggaagtgaaagtaatggaaatgtgccttacctgcagcaaaatacacacactctacctcataatcatacagacctgaacagcagcacagaagagccatggagaaaacagctatctaactccgctcaggggcttcataaaagtcagagttcatgtttgtcaggacctaatgaagaacaacctctgttttccactgggtcagcccagtatcaccaggcaactagcactggtattaagaaggcgaatgaacatctcactctgcctagtaattcagtaccacagggggatgctgacagtcacctctcctgtcatactgctacctcaggtggacaacaaggcattatgtttaccaaagagagcaagccttcaaaaaatagatccttggtgcctgaaacaagcaggcatactggagacacatctaatggctgtgctgatgtcaagggactttctaatcatgttcatcagttgatagcagatgctgtttccagtcctaaccatggagattcaccaaatttattaattgcagacaatcctcagctctctgctttgttgattggaaaagccaatggcaatgtgggtactggaacctgtgacaaagtgaataatattcacccagctgttcatacaaagactgatcattctgttgcctcttcaccctcttcagccatttccacagcaacaccttctcctaaatccactgagcagagaagcataaacagtgttaccagccttaacagtcctcacagtggattacacacagtcaatggagaggggctggggaagtcacagagctctacaaaagtagacctgcctttagctagccacagatctacttctcagatcttaccatcaatgtcagtgtctatatgccccagttcaacagaagttctgaaagcatgcaggaatccaggtaaaaatggcttgtctaatagctgcattttgttagataaatgtccacctccaagaccaccaacttcaccatacccacccttgccaaaggacaagttgaatccacccacacctagtatttacttggaaaataaacgtgatgctttctttcctccattacatcaattttgtacaaatccaaaaaaccctgttacagtaatacgtggccttgctggagctcttaaattagatcttggacttttctctaccaaaactttggtagaagctaacaatgaacatatggtagaagtgaggacacagttgctgcaaccagcagatgaaaactgggatcccactggaacaaagaaaatctggcgttgtgaaagcaatagatctcatactacaattgccaaatacgcacaataccaggcttcctccttccaggaatcattgagagctggaatgcaatggtgtgatctcagctcactgcagcctccgcctcctgggttcaagcgattctcccacctcagcctcccgaatagctggaattacaggcacctgccatcatgcccaactaatttttgtatttttgtagagacagggtttcaccatgttggccaggcttgtcttgaactcctgacctcaggtggtctgcttgcctcagcatcccaaagtgctgggattacaggtgtgagccaccatgcccggtaaacttttaaaaatgtaagcaaaattacagtatgtaaaacacacattgctaatggagaaataaagttcctactttacatctaaaaaaaaaa

SEQ ID NO: 17 - protein, 1079 aa, Homo sapiens
MKSCAVSLTTAAVAFGDEAKKMAEGKASRESEEESVSLTVEEREALGGMDSRLFGFVRLHEDGARTKTLLGKAVRCYESLILKAEGKVESDFFCQLGHFNLLLEDYSKALSAYQRYYSLQADYWKNAAFLYGLGLVYFYYNAFHWAIKAFQDVLYVDPSFCRAKEIHLRLGLMFKVNTDYKSSLKHFQLALIDCNPCTLSNAEIQFHIAHLYETQRKYHSAKEAYEQLLQTENLPAQVKATVLQQLGWMHHNMDLVGDKATKESYAIQYLQKSLEADPNSGQSWYFLGRCYSSIGKVQDAFISYRQSIDKSEASADTWCSIGVLYQQQNQPMDALQAYICAVQLDHGHAAAWMDLGTLYESCNQPQDAIKCYLNAARSKRCSNTSTLAARIKFLQNGSDNWNGGQSLSHHPVQQVYSLCLTPQKLQHLEQLRANRDNLNPAQKHQLEQLESQFVLMQQMRHKEVAQVRTTGIHNGAITDSSLPTNSVSNRQPHGALTRVSSVSQPGVRPACVEKLLSSGAFSAGCIPCGTSKILGSTDTILLGSNCIAGSESNGNVPYLQQNTHTLPHNHTDLNSSTEEPWRKQLSNSAQGLHKSQSSCLSGPNEEQPLFSTGSAQYHQATSTGIKKANEHLTLPSNSVPQGDADSHLSCHTATSGGQQGIMFTKESKPSKNRSLVPETSRHTGDTSNGCADVKGLSNHVHQLIADAVSSPNHGDSPNLLIADNPQLSALLIGKANGNVGTGTCDKVNNIHPAVHTKTDHSVASSPSSAISTATPSPKSTEQRSINSVTSLNSPHSGLHTVNGEGLGKSQSSTKVDLPLASHRSTSQILPSMSVSICPSSTEVLKACRNPGKNGLSNSCILLDKCPPPRPPTSPYPPLPKDKLNPPTPSIYLENKRDAFFPPLHQFCTNPKNPVTVIRGLAGALKLDLGLFSTKTLVEANNEHMVEVRTQLLQPADENWDPTGTKKIWRCESNRSHTTIAKYAQYQASSFQESLRAGMQWCDLSSLQPPPPGFKRFSHLSLPNSWNYRHLPSCPTNFCIFVETGFHHVGQACLELLTSGGLLASASQSAGITGVSHHAR

SEQ ID NO: 18 - DNA, 5270 bp, Mus musculus
  source 1..5270: mol_type = other DNA
attggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagatagcagcttcttcgggttcttgacccgatgcaaagatggcgccaagatgaagacgctgttgaacaaggcaattcacttctatgagtctttaatagtaaaagctgaaggaaaagtggaatcagatttcttttgtcaattaggtcacttcaatctcttactggaagattattcaaaagcattgtcatcttaccagagatactacagtttacagactgactactggaagaatgctgcctttttatatggtcttggtttggtctatttctactacaatgcatttcagtgggcaattagagcttttcaggaagtcctttatgttgaccctaatttttgcagagccaaagaaatccatttacgacttggttttatgttcaaaatgaatacagattatgaatctagtttaaagcattttcagctggccctgatcgactgtaatgtctgtactttgtccagtgtcgaaattcagtttcatattgctcacttatatgaaacccagaggaaataccattctgcaaaggcagcctatgaacaacttttacagattgaaagcttgccatcacaagtcaaagcaactgtattgcaacagttaggttggatgcaccataatatggatctaattggagacaataccaccaaagagagatatgctattcagtatctccaaaagtcattggaggaagatcctaattctggtcaatcttggtatttccttggaaggtgttactcatgtattgggaaggttcaggatgcctttgtatcttacaggcaatccattgataaatcagaagcaagtgcagacacatggtgttcaataggtgtgctgtatcaacagcaaaatcaacctatggatgccttgcaagcctatatatgtgctgtacagttggatcatggacatgcagcagcctggatggacttaggtattctttatgaatcttgcaaccaaccccaggatgccattaaatgctacttaaatgcagctcggagcaaatcttgtaataacacctctgcacttacatcaagaattaagtttttacaggctcagttgtgtaaccttccacaaagtagtctacagaataaaactaaattacttcctagtattgaggaggcatggagcctaccaatacccgcagagcttacctccaggcagggtgccatgaacacagcacagcagagtgtttctgatacctggaatagtgtccagacagcttcacatcattcagtacagcagaaagtttatacacagtgcttcacagcacagaaattacagagttttggtaaagatcaacaacctccatttcaaactgggtccacacgatatctccaagcagctagcactaatgatcagaatcagaatggaaatcatactctgcctcagaattcaaaaggggatgctcaaaatcacttcttacgtattcctacctcagaagaacaaaagatcattaactttaccaaagagagcaaggattcaagaagtaaatccttgacttctaaaacaagtaggaaagatagagacacatctaatatctgtgttaatgcaaagaagcattctaatcatatttatcagatctcatcagttccgatttccagccttaataataaagaatctgtgtcaccagatttaataattgtagacaatcctcagctttctgtcttagttggagaaactatcgacaatgtggaccatgacattggaacttgtgacaaagtcaataatgttcatctagccattcacaaaaagcctgataatctttctgcttcttcaccctcttcagccatttccacagaaacactttctctaaagctcactgaacagacacacattgttaccagctttatcagtcctcacagtggattacacacaattaatggagaagggcatgagaacttagagagttctgccagtgttaatgttggcctcagacctagatctcaaatcataccatcaatgtctgtgtccatatactccagttcaacagaagttctgaaagcgtgcaggagtctaggtaaaaatggtttgtctaatggtcatattctcttggatatatgcccacctccaagaccaccaacttcgccatatcctcctttaccaaaggagaagttgaatccacccactcctagtatttatttggaaaataagcgagatgccttttttcctccattacatcaattttgcatcaatccaaaaaaccctgttacagtaattcgcggtcttgctggagctcttaagttagatcttggacttttctctaccaaaactttggtagaggctaacaatgaacatatcgtagaagtgaggacccaattgttacagccagcagatgaaaactgggatccctctggaacaaagaaaatctggcgatatgaaaataaaagttctcatactacaattgctaaatatgcacagtaccaggcctgctccttccaggaatcattgagagaagaaaatgaaagaagaacacaagttaaagactattcagataatgaatcaacatgttcagataattctggaaggagacagaaagcaccctttaaaaccataaaatgtgggattaacattgatctgtctgataacaaaaagtggaagttgcagttacatgagctgacaaagcttcctgcttttgtacgtgttgtatcagcagggaatcttctcagtcatgttggctatactatattggggatgaattcagttcaactatgtatgaaagttccagggagtagaataccaggtcaccaagaaaataacaatttctgttctgttaacataaatattggtccaggtgattgtgaatggtttgttgtacctgaagattattggggtgttctgaatgacttctgtgaaaaaaataatttgaatttcctgatgagttcttggtggccgaaccttgaagatctttatgaagcaaatgttcctgtttataggtttattcaacgacctggtgatttggtttggataaatgcaggcactgttcactgggttcaggctattgggtggtgtaacaacatcacctggaatgttggtccactcacagccttccaatataaattggcagttgaacgatatgaatggaataaattgcaaagtgtcaagtcagtagtacccatggttcatctttcatggaatatggcacgaaatatcaaagtctcagatccaaagctttttgaaatgattaaatattgtctcctcaagattctaaagcattgtcagactttgagagaagctcttgttgcagcagggaaagaggttttgtggcatgggaggataaatgatgaaccagctccttactgtagcatctgtgaggtggaagtttttaatttactttttgtcactaatgaaagcaattctcaaaagacatatatagtacattgtcagaactgtgcacgaaaaacaagtggaaatttggaaaattttgtagtgctagaacaatacaaaatggaggacctgattcaagtctatgaccaatttacattagctccttcgttatcttctgcatcttaatattgttccagaaatactgaggcgtttgttcttcttgaaatatactacttattttcttgttattctcataagactgctatctcaaagctaaatttatgcaacctttcaagagtagagaatcaggagactaaacagagcattacttctattccaagatttacaaagttgacccaaatgtatttcataattaaaagttaatagtaatagtcacatgatttgtatatatctgccagaaataacattttgacacaaacaaatacctggaaaacaactttttatatctccatttcctggatgacagatttctttatgcttgtaagttaccccaaattttttgtttagaatcaaatagtatcttatttggctacttctttttaagataactaacaaattgaaaaaaaattttttcttttaccaattttttgctaaagtcaaaataatatgaaaaaggtttatattgctctgtcactattgttttgaagtcttgatttcatgtgattacaaaataaaaatattaacagactggtaaaaagattataggttgcatataaataatttcaaagtgatggttttttgttttgttttgaatcttgacacaaactcaaactattcataaatacatatatatatgtgtatatatgtatgtatacatatatgtacatatatatatatatacacatacatacatatatacatatatatgtatattgctggaaaatccttgtgaattgaatgttatttttttctagatttacttgccattaaatactaaggagttgtataattttaaatgccacaattaatttttatatgtgtaaataaagctacttagtattgtacataaactcttattaaaattatttagtgtataaagaatttttcaatgtttgagttttgaagattatatagttccaatctttgttccttttcaaagttaatgaatatatgtatttaagtctgccaacacatgtatgtatgtagagatctcagtatttcactcaacaagaaaatcacaatttttcaattttttatggctgaaagtttttgaaggtatttgcttacaaaataggtatgcttatttttaaagaccatcagtgtcttttagtctgattaggttacaccttatgaaatgacaagtagttgctgtcagaaatattttttaagggtagaaaataaaggtaaattaagatttttttataagaaacatagatttgacaagtgcttatcatttagagttttgctttaatggaaaagttcattgcaatgatgatctctattttagagtttaatattcctaggaacttttaaaacagataattttgaaattccatgtattcctgcttgttcaatctggtgtgtgttgggagacttacgcattaaaatttattattagtaaatcttaaaaaaaaaaaaaaaaaaaaaaa

SEQ ID NO: 19 - protein, 1212 aa, Mus musculus
MKSYGLSLTTAALGNEEKKMAAEKARGEGEEGSFSLTVEEKKALCGLDSSFFGFLTRCKDGAKMKTLLNKAIHFYESLIVKAEGKVESDFFCQLGHFNLLLEDYSKALSSYQRYYSLQTDYWKNAAFLYGLGLVYFYYNAFQWAIRAFQEVLYVDPNFCRAKEIHLRLGFMFKMNTDYESSLKHFQLALIDCNVCTLSSVEIQFHIAHLYETQRKYHSAKAAYEQLLQIESLPSQVKATVLQQLGWMHHNMDLIGDNTTKERYAIQYLQKSLEEDPNSGQSWYFLGRCYSCIGKVQDAFVSYRQSIDKSEASADTWCSIGVLYQQQNQPMDALQAYICAVQLDHGHAAAWMDLGILYESCNQPQDAIKCYLNAARSKSCNNTSALTSRIKFLQAQLCNLPQSSLQNKTKLLPSIEEAWSLPIPAELTSRQGAMNTAQQSVSDTWNSVQTASHHSVQQKVYTQCFTAQKLQSFGKDQQPPFQTGSTRYLQAASTNDQNQNGNHTLPQNSKGDAQNHFLRIPTSEEQKIINFTKESKDSRSKSLTSKTSRKDRDTSNICVNAKKHSNHIYQISSVPISSLNNKESVSPDLIIVDNPQLSVLVGETIDNVDHDIGTCDKVNNVHLAIHKKPDNLSASSPSSAISTETLSLKLTEQTHIVTSFISPHSGLHTINGEGHENLESSASVNVGLRPRSQIIPSMSVSIYSSSTEVLKACRSLGKNGLSNGHILLDICPPPRPPTSPYPPLPKEKLNPPTPSIYLENKRDAFFPPLHQFCINPKNPVTVIRGLAGALKLDLGLFSTKTLVEANNEHIVEVRTQLLQPADENWDPSGTKKIWRYENKSSHTTIAKYAQYQACSFQESLREENERRTQVKDYSDNESTCSDNSGRRQKAPFKTIKCGINIDLSDNKKWKLQLHELTKLPAFVRVVSAGNLLSHVGYTILGMNSVQLCMKVPGSRIPGHQENNNFCSVNINIGPGDCEWFVVPEDYWGVLNDFCEKNNLNFLMSSWWPNLEDLYEANVPVYRFIQRPGDLVWINAGTVHWVQAIGWCNNITWNVGPLTAFQYKLAVERYEWNKLQSVKSVVPMVHLSWNMARNIKVSDPKLFEMIKYCLLKILKHCQTLREALVAAGKEVLWHGRINDEPAPYCSICEVEVFNLLFVTNESNSQKTYIVHCQNCARKTSGNLENFVVLEQYKMEDLIQVYDQFTLAPSLSSAS

SEQ ID NO: 20 - DNA, 1784 bp, Homo sapiens
  source 1..1784: mol_type = other DNA
acttccttctttcgtgagcaagatggcgggcggagaagccggtgtcactctcgggcagccgcatctttctcgtcaggatcttgccactttggatgttaccaagttgactccgctttcacgtgaaattatcagcagacaagccacaattaatataggcacaattggtcatgttgctcatggaaaatctacagttgtaaaagccatttctggtgttcacactgtccgattcaaaaatgaactagaaaggaatattaccataaaacttggatatgctaatgccaaaatttataagcttgatgactcaagttgtcctcgaccagaatgttacagatcttgtggaagtagtacacctgatgagtttccttcagatattccagggaccaaaggaaacttcagactagtcagacatgtttcctttgttgattgtcctggtcatgatattttgatggcaactatgctgaatggggcagcagtgatggatgcagctcttctgttgatagctggtaatgaatcttgtcctcaacctcagacttctgaacacctggctgccattgaaattatgaagctaaaacatattttgattctgcaaaataaaattgatttggtgaaagaaagccaggctaaagaacagtatgaacagatacttgcatttgtacagggtacagtagccgaaggagctcctattattccaatttctgctcagttaaaatacaatattgaagttgtatgtgagtatatagtaaagaaaattccagtacctctaagagactttacttcagaaccccgacttattgttattcggtcttttgatgttaacaaacctggctgtgaagttgatgaccttaaagggggtgtagctggtggtagtattttaaaaggcgtattaaaggtgggacaagagatagaagtgagacctggtattgtttctaaagacggagaagggaagcttatgtgtaaaccaatcttttccaagattgtatccctttttgcagaacacaatgatcttcagtatgctgctccaggtggtcttattggagttggaacaaaaattgacccaacgttatgccgagcagatagaatggttgggcaggtccttggtgctgttggagcattacctgagatattcacagagttagaaatttcctacttcctactgagacggctcctaggtgtacgtacagaaggagacaagaaagcagcaaaagttcaaaagctatccaagaatgaagtactcatggtgaacatagggtccttgtctacaggaggcagagttagtgcagtcaaggcagatttgggtaaaattgttctaaccaatccagtatgcacagaagtaggagaaaaaattgctctaagccgacgagttgagaaacactggcgtttaattggttggggccagataagaagaggcgtgactatcaagccaacaatagatgatgaatgagatgatacatttggatgaaaccaggaattttttcataacagtcttagagatattttcaaatctaaaaagtaggacatctaaagaaggaaataaatttcacagcttattattatattagtaaaaattgccaggttattttcattttcagtagaaaaaaattgatgtctgtaaaaaaaaatgacaaacttcttgatacatttaatctgcaattcaacaaatagtattattgttgtagcttgatagttaacaactgttgctgtgcaccactgaacttacagtaaagaaagtttttcttatgtttgaaggattgttactctaataaaaacttgaaggctggattcaa

SEQ ID NO: 21 - protein, 472 aa, Mus musculus
MAGGEAGVTLGQPHLSRQDLATLDVTKLTPLSREIISRQATINIGTIGHVAHGKSTVVKAISGVHTVRFKNELERNITIKLGYANAKIYKLDDSSCPRPECYRSCGSSTPDEFPSDIPGTKGNFRLVRHVSFVDCPGHDILMATMLNGAAVMDAALLLIAGNESCPQPQTSEHLAAIEIMKLKHILILQNKIDLVKESQAKEQYEQILAFVQGTVAEGAPIIPISAQLKYNIEVVCEYIVKKIPVPLRDFTSEPRLIVIRSFDVNKPGCEVDDLKGGVAGGSILKGVLKVGQEIEVRPGIVSKDGEGKLMCKPIFSKIVSLFAEHNDLQYAAPGGLIGVGTKIDPTLCRADRMVGQVLGAVGALPEIFTELEISYFLLRRLLGVRTEGDKKAAKVQKLSKNEVLMVNIGSLSTGGRVSAVKADLGKIVLTNPVCTEVGEKIALSRRVEKHWRLIGWGQIRRGVTIKPTIDDE

SEQ ID NO: 22 - DNA, 4640 bp, Mus musculus
  source 1..4640: mol_type = other DNA
ccaaaactgtgtacgtaagctcttgcgttcaccgtgaggaaacaggtctacggtggctgttccgtgagaagttctcaatttttttcagcaatgagtcaagtggcagcggaaagtacggccggcctggaccagcaatttgttggtcttgacctgaaatcatcagataatcagaatggaggaggaaatacagagagcaaagggcgctatatacctcctcacttaaggaacagagaaacatctaaaggggtctgtgataaggacagttcaggatggagctgtagtaaagataaagatgcctacagcagttttggatctcgtgattccagagggaagcccaattatttcagtgatcgtggaagtggatccaggggaaggttcgatgatcatggtcgaaatgactatgatggtattggtggtcgtgaccgaactggatttggcaaatttgaacggagtggtcatagtcgttggagtgacagatcagatgaagatgactggtcaaaaccacttccaccaagtgaacgcttagagcaagaacttttttctggaggaaacacagggattaactttgagaaatatgatgatataccagtagaagcaactggaaataattgtcctcctcacattgaaaatttcagtgatattgagatgggagaaattatcatggggaacattgaacttactcgttacactcgtcctactccagtgcaaaaacatgctattccaattattaaagaaaaaagggacttaatggcttgtgctcaaacagggtctggaaaaactgctgcatttctactacccattctcagtcagatttatacagatggtccaggagaggctttgaaggctatgaaggaaaatggaagatatggtcgccgtaaacaatacccaatctccttggtgttagccccaacaagagaattggctgtgcagatctatgaggaagccagaaaattttcataccgatctagagtccgtccttgtgtagtgtatggtggtgctgatactgttcagcagattcgggacttagaacgtggatgccacttgttagttgccacaccaggacgtctagtggacatgatggaaagaggaaagattggattagatttctgcaaatacttagtattggatgaagctgataggatgctggatatgggatttgaacctcaaatacgtcgtatagttgagcaggacacaatgccaccaaagggggttcgtcacactatgatgtttagtgctacttttcctaaggagatacagatgcttgctcgtgactttttagatgaatatatctttctggctgtaggcagagtaggatctacttctgagaacatcacacagaaagtagtatgggttgaagaactagacaaacggtcatttttacttgatctcttaaatgcaacagggaaggattcattgactttagtatttgtggagactaaaaagggtgcagattcgctggagaacttcttatttcaagaaagatatgcgtgtactagtattcatggagacagatcacagaaagatcgagaggaggcccttcaccagtttcgctcagggcgaaagccaattttagtggctacagctgtggcagcaagaggtctagacatttcaaatgtgaaacatgttatcaattttgatttgccaagcgatattgaagaatatgtgcatcgaattggccgcacaggacgtgtaggaaaccttggacttgccacctcttttttcaatgagaggaatttgaacatcacaaaagatttgttagatctccttgttgaggctaaacaagaagtgccttcttggttggaaagtatggcttatgaacaccactacaagggaagtagccgtggacgttctaaaagcagattcagtggaggatttggagccagagactatcgacagagcagtggttctgcaaatgctggatttaatagtaaccgtgccaatagcagccgaagtagtggtagtagccacaacagaggatttggtggaggtggctatggaggtttctacaataatgatggttatggaggaaattataactcccaggcagttgactggtggggcaattgaatttgtatcattatttcatacagctaatatggaaaccacatgtaacttagccagactgtattgtgtagcttcaagaacttgcagtacattaccagctgtgattcttctaaaagttcatgagagctcaaagtcacaagaaattaagcaacaacctgtagccctattcaaagttgatgagaaaaatttttgtaactggattaattcacttcctgcttggttgtaccctaaacagcatttataatttgtgagtgaggatcatttgttaatgtactatgacttttaactttaaacaacttatttaatgtcctgttgtctcaataatgctcaagatataaattgttttaataagttaattgaattttggctacaagcaaaccttggcacacaagtgtaatacaccttaataggaatcaattcattgctaagtatgatggtgaatgaacttatcctggataataaatttttggtatatgcagagttgttggtaaaacttcttttttaagtttgtaatgtggcagacatacaaaactaaagttctgaactgctcactatatcctgcattgacacttagaaccttaaagtattaaatatatgattatatatggttatatcctttattttcaaaagcactaatgcagataactacagtgattcctcatttactgttttaagtaatttatattggcatactcctacatgaaagtttaaaattttggtgaagaatttgggatataaagtttaggtatatttaatgcctgccatgcataaaaaatggtaaattactaggtgactatggcaatgaccagaccttattaaagctttgttaattctggtctttaatgcatgctagtgttggtgttttttggtcaagagagtatgaacagattatgcagcagcaggctttacttttacacagtcaggtttttctgtaacaactatgttgaaatgtaaaaataaaataaaataaaataaaaaaggcttacatacatactatgtaaatataagaataaaaaattgaaattgctgagatctcacaggttggatttatgtactaattatacatgatggtcagtacagtgcagttttcatctattttcgttgcataggtttccattgtattatagtcatattttaattggccaaaaatgattgtccctcacaaaaaatgccttttcctttgagaatttcctggaaagaatacaataaatttaaaaacgtaagtccttaaaaatactataagttgataaatttttctaaaagtgcaatagattttcaagtgtattgtgccttgtattaaaattttaaagtaggtgtacttgacagtattatcaaggtcatttgttacggtgctatttcaattatagaagtttagactgtacattgcccataactttttacaaagtacttattttattgcacatgaagagaattttatatgtgtatcttatgtacaagtccttgaagttccaattggatattgttgattacagtgtgtcaagaacttggttcagaaaattttccatggaccatagaatgatagttaagaaaaactatttgcagcctgaagtggtggcacacgcctttaattgcagcactcaggaggcagaggcaggtggatttctgaatttgtggccagcctgttctgcatagtgagttccaggacagctagggctctacagagaaaccttgtctcaaaaaaccaaaaaaaagaaaaaaaagaaaaagaaaaaaaagaaagaaaaactatttgcacataacagactttttgctgctaggttgtgtaatatttattgaacattttgacaaatatttatttttgtaagcctaaaaatgattctttgaaagcttaaagaaacttgaccaaaagacagtattaaaaaaaacactggtatttgattgtcactgtatgtgaaatagtatattttgggtaatgtgagctttttaatgttaagttctactaaacttgagtcaaattgatctaacccagtattagtgatgtagctacactatttttctttttttcttttgacaaaatgaaaggcaattggaataatgaaaatttaaaacatgccaaggttttgatgtatttaagatattagtgaaaactaaaaattttctcagtttgtttgaatttaccttacttacatgatctatttgttcagtattgtaactgtctggcatatttataatcttcaaacatgtggttatatctttgtattgccataatcagtgatgttttacattcagtttatcagccagcaatactttcagtaaataatacactaaatttgctgactgtagacatggaactttaagtttctataaagttagtaattgctttattgtattagtattaggtgctagcactgcatatgtctgatctattctcattttattaaaataaaaagtatagcaaaatgtttactgtaacttgtgacttaatgttcttgtgaacttgcatgcttgaactgttggaaatctcatttttagttaataaaagaaggtatttgtaaagtacactgttatgtatgtgtttgctttttagttaataaaagaaggtatttgtaaagtac

SEQ ID NO: 23 - protein, 658 aa, Mus musculus
MSQVAAESTAGLDQQFVGLDLKSSDNQNGGGNTESKGRYIPPHLRNRETSKGVCDKDSSGWSCSKDKDAYSSFGSRDSRGKPNYFSDRGSGSRGRFDDHGRNDYDGIGGRDRTGFGKFERSGHSRWSDRSDEDDWSKPLPPSERLEQELFSGGNTGINFEKYDDIPVEATGNNCPPHIENFSDIEMGEIIMGNIELTRYTRPTPVQKHAIPIIKEKRDLMACAQTGSGKTAAFLLPILSQIYTDGPGEALKAMKENGRYGRRKQYPISLVLAPTRELAVQIYEEARKFSYRSRVRPCVVYGGADTVQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEELDKRSFLLDLLNATGKDSLTLVFVETKKGADSLENFLFQERYACTSIHGDRSQKDREEALHQFRSGRKPILVATAVAARGLDISNVKHVINFDLPSDIEEYVHRIGRTGRVGNLGLATSFFNERNLNITKDLLDLLVEAKQEVPSWLESMAYEHHYKGSSRGRSKSRFSGGFGARDYRQSSGSANAGFNSNRANSSRSSGSSHNRGFGGGGYGGFYNNDGYGGNYNSQAVDWWGN

SEQ ID NO: 24 - DNA, 5471 bp, Mus musculus
  source 1..5471: mol_type = other DNA
acattgtgatctaaaaatggtggaaccgaacgaggaggtgaatatttgtaagaatgtggaattaagcaaactctgaaatttagaagaaaataaagttcctgggtgccacgcagtgagtaggcaattccattcgccctttaatatcgaatcactttttataaccgcgttttgagcgaacatgaagccaggatctgacgactttctaccgccgcctgagtgccctgtgtttgagcccagttgggcggaattccgcgatcctcttggctatatagctaaaatcagacccattgcggagaaatcaggcatttgcaagatacgcccacctgcggattggcaacctccttttgctgtagaagttgataacttcagatttactcctcgaattcagaggctaaatgaactggaggcccagaccagagtaaaactgaactatttggatcagattgcaaagttctgggaaattcaaggttcttccttaaagatacctaatgtggagaggaagatcttggacctttacagccttaataagattgtgatggaggaaggtggatatgaagcaatctgcaaggaccgtcggtgggcccgtgttgcccaacgccttaattacccatcagggaaaaacattggttccctgcttcggtcccactacgaacggatcatttacccttatgaaatatttcagtctggagctaatcttgtgcaatgtaacacagatccatttgacagtgaagaaagagacaaggaatacaaacctcatagcattcctcttagacagtctgttcagccatcaaaattcagttgctacagtagacgaggaaaaaggctacagcctgagccagaacccacagaagaagacattgagaaaaacccagagctaaagaaactacagatatatggggcaggccccaagatgataggcttgggcctcaaggctaaggagaaaactctgagaaagaaagatagcaaacaaccagataaagaagaagtgacatgccctgcaactattgtagtgaagggggaagccagtgagtttggaaaggtgacatcagcattttcagacaagaacttaaatcacagttttgaaccttgtatgaagatgactatgcagctacggaacaatcacagcagtacccagtttatgaattcatatgtttgccgaatatgctctcgtggggatgaagtcgataagtttctgctctgtgatggctgcagtgataattaccacatcttttgcctattgccaccgctttctgaagtccccaagggagtatggagatgcccgaagtgtatcttagcagagtgtaagagtccccctgaagcttttggctttgagcaggctacacaggagtatactttgcagagttttggtgagatggctgactccttcaaggctgactacttcaacatgcctgtgcatatggtacctacagaagttgtggaaaaggaattctggaggctggtgagcagcattgaggaggatgtgacagttgaatatggagcagacattcactccaaagaatttggcagtgggtttcctgtcaacaatagcaaatgggacttatctcctgaagaaaaggaatatgctgcttgtggttggaatctcaatgtgatgccagtgctggatcagtctgttctctgccacatcaatgcagacatctcaggcatgaaagtgccctggttatatgtgggcatggtgttttcagcattttgttggcatattgaggatcactggagttattccattaactacctgcactggggtgaaccaaagacctggtatggagtaccttcgctagcagcagaacacttagaggatgtaatgaagagacttacaccagagttgtttgacagccaacctgacctcctgcaccaacttgtcactctgatgaatcctaacactctcatgtcacatggagtgccagttgttcgtacaaaccagtgtgcaggggaatttgtcattacttttccccgtgcttaccacagtggctttaaccaaggctataactttgctgaggcagtgaacttttgcactgccgattggctacctgttggacgccagtgtattgaacactaccgccgactccggcgctattgtgtcttctcccatgaagaacttatctgcaagatggctgcatttccagagaagttggatctaaatctagcagtggcagttcacaaggagatgttcattatggtacaggaggagcgacgcctacgaaagaccttgttggagaagggcatcacggaagctgagcgagaggcttttgagctactcccagatgatgagcgtcagtgcatcaagtgtaagaccacgtgtttcctatcagccttggcctgttatgactgtccagatagtcttgtctgcctttcccatatcaatgatctgtgcaagtgctcaaggaatcgacagtacttacggtacaggtacacattggatgaactgcctgctatgctacaaaagctgaagattcgagctgagtcatttgacaactgggccaacaaagtacaagcagccctagaggtggaggatggtcggaaacggagttttgaagagctaagagcattggaatctgaggctcgtgatagacgatttcctaatagtgagctgcttcagcgattgaagaaatgcctaactgaagcagaggcttgtatttcccaagttcttggactgatcagtaactcagaggacaggttacaaacaccgcaaatcacccttactgaactacagcttcttctcaaacagatgggcactctgccctgtaccatgcatcagattgatgaagttaaggatgttcttcaacaggtggaatcataccaaattgagacccgtgaggctctaacttcactgccttacagtttagaaatacttcagtccctgatggaaaagggtcagcagcttcgtgtggaagtgcctgaagctcatcaacttgaagagctgttggagcaggcacaatggctagatcaagtgaagcaggccctggctccctcaggccagaggcactctttggttatcatgaagaagcttttagttatgggaaccaaggtagcctccagcccttctgtgaacaaagctcgagcagagttacaagaactgctgactattgcagaatgttgggaggaaaaggcccatttttgcttaaaagctagccaaaagcattctccagccactttggaagttataatacgtgaggcagaaaacatccctgtctacctacctaacatccagtctctgaaggaagctctgactaaggcacaggcttggattgctgatgtgaatgagattcaaaatggtgatcactacccctgtttggatgatttggagggccttgtagctgttggtcgggatttacctgtggagctagaggaattgagacagctggagaatcaggtactaacagcacattcctggaaagagaaagcttctaagacttttcttaagaagaattcttgctacacattgttggaggttctctgcccatgtgcagatgctggttcagtcagcaccaagcgcagtcggtggattgaaaaggagatgggattatacaaatatgacacagaattgctggggctgtctgcacaggacctcagggacccagggtctgtgatcatggcctttaaggaaggggaagagaaagaaaaggaaggtatcttgcacttacgtcacatcaactcagccaagcccagtccaatgtcctcatcgatgaatgcctctgcaacctccatctgtatatgtgggcaggtctgtgcaggagtggaatctctgcagtgtgatctttgtcatgactggttccatggacaatgtgtgactgtaccccacctactcagctcagtaagggccagtcatacctcatctcaactgctagcttggtgggaatgggacaccaagttcctgtgcccactgtgcatgcgatcaaggcgtccacgcctggagaccatcttgtcattgctggtaggactacagagactgtctgtaaggctacctgagggtgaggccctacagtgcctcacagagagagccattggttggcaaggccgtgccaggcaggctctagcttctgaggatgtgactgctttgttgaaacagctggaaaaatcccgccaacaactacaggatgaattgagacataagaagccccctaccttgccttcaggctttgcctttgactgtctcacagagaatagtggcaaggatatccttaaggaagaggaggagttggtactaaatgaagaaaggattaaaagttctgagaagattgttccaaaggaaagttcatgtaaaggagataaggaactgctgccttcacttttatcccaattaactggcccagtactggaactgcctgaagccacccgggcacccctagaggagcttatgatggaaggagatctacttgaagttaccctagatgagaactacagcatttggcagctgctgcaggctgggcagaatcccaatctagagcgcattcatacacttctggagcttgagaagcctgaaaacccaggaaattggtcagaggaacagacaccagagaggcgacggcagaggaggcaaaaggtggttctgagtagaaagggtgaggattttactcaaaaagagctagaatcaaaaagagtcaagagttcaaggattaagcctaaggaagaaaagtttcaaaagccaatacttggggataatgtgctgtacacacaccatacagaacatacaaacatcttaaaagagcacataaacagtgtacaaggaaaggatccaagcccttcatcttcttttccttccttaacacctttactacacctatcttattttcatcagcaaaaattataacaatagtatgtgattataacaatagtatgtgattgttcttttttcctaccttagtcttttccattctccctcccttgttctttctttccttcttctttttcatttctttctgttggtcccttcctttcctccattttactctattcctacagcttcctctgcccttaatccctcagatacccccaaagtatttgtttcttatctagagcttgaccacaaattattttatccttactcctaatacatttttatgccataattcctaagaagtattccctgtatgcgtttcccaaaattttaccatcaatattcctgggactagcatagataaagtgatgttcttagttatctctattctaatctggagagacaaaaataaaatttgagaatgataacatttattaatctttatttttaaatccttcatcaatcacacatcaaaacaagtacttattaaaacaagtgtcttttagtttataattttcattctatttttctctttggtttacattctaaaagaacgttgtatttttaattcttaagaaaaaggaatatatacagtaatgcaaacttaccataattgtttatttttaattttgttccttttgattttgttgttaaaaataaaacagtatggttagaactg

SEQ ID NO: 25 - protein, 658 aa, Mus musculus; identical to SEQ ID NO: 23

SEQ ID NO: 26 - DNA, 2780 bp, Homo sapiens
  source 1..2780: mol_type = other DNA
gccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatgcagggactctgataacacccattttaaaggtgaggaaacaagcccagagaggttaagggaggagttcctgcccaccaggaacctgctttagtgggggatagtgaagaagacaataaaagatagtagttcaggccaggcggggtggctcacgcctgtaatcctagcacttttgggaggcagagatgggaggattacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 27 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 28 - DNA, 2191 bp, Mus musculus
  source 1..2191: mol_type = other DNA
cgccgccgccgccgcccttcgcgccccaggccgtccccctcctcctcccgccgcggatcctccagacagccaggcccccggccggggcaggggggacgccccttcggggcacccccggctctgagccgcactcggagtcggcctccgctgggagccggcaaaggagcagccgaggagccgtccgaggccccagagtctgagaccagccgccgccgcagggaggagggggaggaggagtgggaggagggacgagctggttgagagaagaggaaaaaagttttgagacttttccgctgctactgcaagtcagagacgtggggacttcttggcactgcgctgtctcgcaaggaggcaggacctgaggactccagacagccctgctcaccgtcgtggacactcgatcgctacccggcgttcctcagacgcccctattccggaccagccctcgggagccacaaaccccgcctcccgcgaagacttcaccccaaagctggggcgcaccccttgcacgccgccctccccccagcctgcctcttgagtccctcgcatcccaggaccctctctcccccgagaggcagatctccctcggacctgctggcagtagctcccctatttaagaacacccacttttggatctcagagagcgctcatctcgatttttaccctggtggtatactgagacaccttggtgtcagagcctcaccgcgactcctgctgctttctccctcaacctcaaattattcaggactatcacctacctttccttgggagaccccaccccacaagccctgcaggggcggggcctccgcatcccacctttgccgagggttcccgctctccgaagtgccgtggggcgccgcctcccccatgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctgaagccccgccccgccccgcccctcccggcaggcccggccccgcccccgccccgcccgcagcgcccggggctgtatttaaggacacctgcaccccccctcaagcccacctggggggcccattaaaggtgacagaggaaaaaaaaaaaaaaaaa

SEQ ID NO: 29 - protein, 390 aa, Mus musculus
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

(19) United States
(12) Patent Application Publication
(10) Pub. No.: US 2025/0297310 A1
(43) Pub. Date: Sep. 25, 2025

(54) HEMATOPOIETIC LOSS OF Y CHROMOSOME LEADS TO CARDIAC FIBROSIS AND DYSFUNCTION AND IS ASSOCIATED WITH DEATH DUE TO HEART FAILURE

(71) Applicant: University of Virginia Patent Foundation, Charlottesville, VA (US)

(72) Inventors: Kenneth Walsh, Charlottesville, VA (US); Soichi Sano, Hikone City (JP)

(73) Assignee: University of Virginia Patent Foundation, Charlottesville, VA (US)

(21) Appl. No.: 18/849,053

(22) PCT Filed: Mar. 21, 2023
(86) PCT No.: PCT/US2023/064786

§ 371 Date: Sep. 20, 2024

Related U.S. Application Data
(60) Provisional application No. 63/322,165, filed on Mar. 21, 2022.

(51) Int. Cl. C12Q 1/6883; A61K 31/4412; A61K 35/17; A61K 40/11; A61K 40/31; A61K 40/42; A61K 45/06; C07K 16/22; C12Q 1/686

(52) U.S. Cl. CPC C12Q 1/6883; A61K 31/4412; A61K 35/17; A61K 40/11; A61K 40/31; A61K 40/4247; A61K 45/06; C07K 16/22; C12Q 1/686; C12Q 2600/158

(57) ABSTRACT

Provided are methods for identifying subjects at risk for reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function. In some embodiments, the methods include determining if the subject has mosaic loss of chromosome Y in blood (mLOY). Also provided are methods for preventing and/or treating diseases, disorders, and/or conditions associated with mLOY, which in some embodiments also include administering an anti-fibrotic therapy to the subject. Also provided are uses of inhibitors of TGFβ signaling or senolytic agents for prevention and/or treatment of diseases, disorders, and/or conditions associated with mLOY.

### CROSS REFERENCE TO RELATED APPLICATIONS

The presently disclosed subject matter claims priority to and the benefit of U.S. Provisional Patent Application Ser. No. 63/322,165, filed Mar. 21, 2022; the disclosure of which is incorporated herein by reference in its entirety.

### GOVERNMENT INTEREST

This invention was made with government support under Grant No. AG073249 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 57,609 byte UTF-8-encoded XML file created on Mar. 21, 2023, and entitled “3062_182_PCT.xml”. The content of the Sequence Listing XML submitted via Patent Center is incorporated herein by reference in its entirety.

### TECHNICAL FIELD

The presently disclosed subject matter relates generally to compositions and methods for preventing and/or treating diseases, disorders, and/or conditions associated with mosaic loss of chromosome Y in blood (mLOY) in subjects in need thereof. In some embodiments, the presently disclosed methods comprise, consist essentially of, or consist of administering to the subject an inhibitor of TGFβ signaling via a route and in an amount sufficient to inhibit TGFβ signaling in the subject and/or administering an effective amount of a senolytic agent to the subject.

### BACKGROUND

The human male-specific Y chromosome is relatively small in size and contains a limited number of genes that regulate sex-determination and spermatogenesis (Jobling & Tyler-Smith, 2017). Beyond sex-determination, there is a paucity of information about the biological role of Y chromosome partly due to challenges in determining genetic variation caused by the inter- and intra-chromosomal repeat sequences. However, insights into the physiological role of the Y chromosome can be aided by studies that address the mosaic loss of chromosome Y in blood (mLOY), a condition where a fraction of hematopoietic cells display loss of the Y chromosome. This phenomenon is the most prevalent postzygotic mutation in leukocytes (Forsberg et al., 2014). The frequency of hematopoietic mLOY increases with age and smoking status (Dumanski et al., 2015; Thompson et al., 2019), and it is associated with the condition of clonal hematopoiesis of indeterminate potential (CHIP; Zink et al., 2017; Ljungstrom et al., 2022). While the technology to assess mLOY is evolving, it has recently been reported that mLOY is detectable in 40% of 70-year-old males and 57% in 93-year-old males (Forsberg et al., 2019; Thompson et al., 2019).

Loss of the Y chromosome is prevalent in hematologic malignancies and may be a factor in the prognosis of these diseases. While most males with mLOY never progress to a hematologic cancer, epidemiological studies have shown that mLOY in blood is associated with shorter lifespan (Forsberg et al., 2014; Loftfield et al., 2018) and increased incidence of various age-associated diseases including solid tumors and Alzheimer's disease (Forsberg et al., 2014; Dumanski et al., 2016). Furthermore, mLOY has previously been associated with secondary major cardiovascular events in atherosclerotic patients after carotid endarterectomy (Haitjema et al., 2017) and prior heart attack and stroke that was self-reported at baseline in the UK Biobank study (Loftfield et al., 2018). It has been reported that mLOY is in part a manifestation of inherited genomic instability and a marker of biological aging (Thompson et al., 2019). However, due to descriptive nature of epidemiological studies, the causal role of mLOY in disease development is largely unknown.

Disclosed herein is the modeling of hematopoietic mLOY in mice and an examination into its role in fibrosis and cardiac dysfunction. The presently disclosed results suggest that mLOY in leukocytes is a causal risk factor for heart failure.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to methods for identifying a subject at risk for reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function. In some embodiments, the methods comprise, consist essentially of, or consist of determining if the subject has mosaic loss of chromosome Y in blood (mLOY), wherein the presence of mLOY in the subject is indicative of the subject being at risk for reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function.

In some embodiments, the determining comprises assaying gene expression in macrophages isolated from the subject, optionally from the subject's blood or heart, for mLOY or comprises detecting a presence or absence of a Uty gene in sample from the subject.

In some embodiments, the determining employs RT-PCR analysis of RNA isolated from a cell isolated from the subject, optionally wherein the cell is a macrophage.

In some embodiments, the gene expression assayed comprises TGFβ1 gene expression or Uty gene expression.

The presently disclosed subject matter also relates in some embodiments to methods for preventing and/or treating a disease, disorder, and/or condition associated with mosaic loss of chromosome Yin blood (mLOY) in a subject in need thereof. In some embodiments, the methods comprise, consist essentially of, or consist of administering to the subject an inhibitor of TGFβ signaling via a route and in an amount sufficient to inhibit TGFβ signaling in the subject and/or administering an effective amount of a senolytic agent to the subject, whereby a disease, disorder, and/or condition associated with mosaic loss of chromosome Y in blood (mLOY) in the subject is treated.

In some embodiments, the inhibitor of TGFβ signaling is an anti-TGFβ antibody or a fragment thereof that binds to a TGFβ polypeptide to inhibit TGFβ signaling in the subject; a nucleic acid molecule that binds to and inhibits expression of a TGFβ gene product in the subject; a small molecule inhibitor of TGFβ signaling, optionally pirfenidone; or any combination thereof.

In some embodiments, the senolytic agent is selected from the group consisting of a FOXO4-related peptide, a BCL-2 inhibitor, a Src inhibitor, a USP7 inhibitor, N-(2-chloro-6-methylphenyl)-2-[[6-[4-(2-hydroxyethyl)-1-piperazinyl]-2-methyl-4-pyrimidinyl]amino]-5-thiazole carboxamide monohydrate and 2-(3,4-dihydroxyphenyl)-3,5,7-trihydroxychromen-4-one, Fisetin (3,3′,4′,7-tetrahydroxyflavone), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-trifluoromethanesulfonyl)benzene-1-sulfonyl)benzamide, azithromycin and roxithromycin, a senescence-specific killing compound 1, (2R,3S,4S,5R,6S)-2-(acetoxymethyl)-6-(4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)-2-nitrophenoxy)tetrahydro-2H-pyran-3,4,5-triyl triacetate, a BIRC5 inhibitor, a glutaminase-1 (GLS1) inhibitor, an anti-Glycoprotein Nmb (GPNMB) vaccine, a cardiac glycoside, 25-hydroxycholesterol (25HC), (2R,3R,4S)-2-(3,4-dihydroxyphenyl)-4-[(2R,3R)-2-(3,4-dihydroxyphenyl)-3,5,7-trihydroxy-3,4-dihydro-2H-chromen-8-yl]-8-[(2R,3R,4R)-2-(3,4-dihydroxyphenyl)-3,5,7-trihydroxy-3,4-dihydro-2H-chromen-4-yl]-3,4-dihydro-2H-chromene-3,5,7-triol, (3E,5E)-3,5-bis[(2-fluorophenyl)methylidene]piperidin-4-one, a heat shock protein 90 (HSP90) inhibitor, and any combination thereof.

In some embodiments, the disease, disorder, and/or condition associated with mLOY in the subject is a reduced lifespan, age-associated cardiomyopathy, reduced cardiac function, heart failure, increased fibrosis, optionally increased fibrosis of the myocardium, lung, and/or kidney; idiopathic pulmonary fibrosis (IPF); elevated left ventricular filling pressure (E/e′) indicative of diastolic dysfunction; and/or reduced cognitive function in the subject.

In some embodiments, the presently disclosed methods further comprise, consist essentially of, or consist of administering an anti-fibrotic therapy to the subject.

In some embodiments, the anti-fibrotic therapy comprises administering to the subject an effective amount of a small molecule anti-fibrotic, an anti-fibroblast antibody or a fragment thereof that binds to a polypeptide expressed by a fibroblast; a nucleic acid molecule that binds to and inhibits expression of a gene product expressed by a fibroblast in the subject; or any combination thereof.

In some embodiments, the small molecule anti-fibrotic is a withanolide compound (a group of naturally occurring steroids built on an ergostane skeleton), a fused ring derivative of 2-{[(2E)-3-(3,4-dimethoxyphenyl)prop-2-enoyl]amino}benzoic acid, tranilast (n-[3,4-dimethoxycinnamoyl]anthranilic acid), metabolites thereof, precursors thereof, or any combinations thereof.

In some embodiments, the anti-fibrotic therapy comprises an anti-fibroblast CAR-T cell therapy, and further wherein the anti-fibroblast CAR-T cell therapy employs CAR-T cells that are directed to fibroblast activation protein (FAP).

In some embodiments, the presently disclosed subject matter also relates to uses of inhibitors of TGFβ signaling for prevention and/or treatment of diseases, disorders, and/or conditions associated with mLOY. In some embodiments, the diseases, disorders, and/or conditions associated with mLOY are selected from the group consisting of age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and reduced cognitive function in a subject, as well as any combination thereof.

In some embodiments, the presently disclosed subject matter also relates to method for preventing and/or treating a disease, disorder, and/or condition associated with mosaic loss of chromosome Y in blood (mLOY) in a subject in need thereof, the method comprising: determining if the subject has mosaic loss of chromosome Y in blood (mLOY), wherein the presence of mLOY in the subject is indicative of the subject being at risk for reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function; and administering to the subject an inhibitor of TGFβ signaling via a route and in an amount sufficient to inhibit TGFβ signaling in the subject and/or administering an effective amount of a senolytic agent to the subject, whereby a disease, disorder, and/or condition associated with mosaic loss of chromosome Y in blood (mLOY) in the subject is treated.

In some embodiments, the determining comprises assaying gene expression in macrophages isolated from the subject, optionally from the subject's blood or heart, for mLOY or comprises detecting a presence or absence of a Uty gene in sample from the subject.

In some embodiments, the determining employs RT-PCR analysis of RNA isolated from a cell isolated from the subject, optionally wherein the cell is a macrophage.

In some embodiments, the gene expression assayed comprises TGFβ1 gene expression or Uty gene expression.

In some embodiments, the inhibitor of TGFβ signaling is an anti-TGFβ antibody or a fragment thereof that binds to a TGFβ polypeptide to inhibit TGFβ signaling in the subject; a nucleic acid molecule that binds to and inhibits expression of a TGFβ gene product in the subject; a small molecule inhibitor of TGFβ signaling, optionally pirfenidone; or any combination thereof.

In some embodiments, the senolytic agent is selected from the group consisting of a FOXO4-related peptide, a BCL-2 inhibitor, a Src inhibitor, a USP7 inhibitor, N-(2-chloro-6-methylphenyl)-2-[[6-[4-(2-hydroxyethyl)-1-piperazinyl]-2-methyl-4-pyrimidinyl]amino]-5-thiazole carboxamide monohydrate and 2-(3,4-dihydroxyphenyl)-3,5,7-trihydroxychromen-4-one, Fisetin (3,3′,4′,7-tetrahydroxyflavone), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-trifluoromethanesulfonyl)benzene-1-sulfonyl)benzamide, azithromycin and roxithromycin, a senescence-specific killing compound 1, (2R,3S,4S,5R,6S)-2-(acetoxymethyl)-6-(4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)-2-nitrophenoxy)tetrahydro-2H-pyran-3,4,5-triyl triacetate, a BIRC5 inhibitor, a glutaminase-1 (GLS1) inhibitor, an anti-Glycoprotein Nmb (GPNMB) vaccine, a cardiac glycoside, 25-hydroxycholesterol (25HC), (2R,3R,4S)-2-(3,4-dihydroxyphenyl)-4-[(2R,3R)-2-(3,4-dihydroxyphenyl)-3,5,7-trihydroxy-3,4-dihydro-2H-chromen-8-yl]-8-[(2R,3R,4R)-2-(3,4-dihydroxyphenyl)-3,5,7-trihydroxy-3,4-dihydro-2H-chromen-4-yl]-3,4-dihydro-2H-chromene-3,5,7-triol, (3E,5E)-3,5-bis[(2-fluorophenyl)methylidene]piperidin-4-one, a heat shock protein 90 (HSP90) inhibitor, and any combination thereof.

In some embodiments, the disease, disorder, and/or condition associated with mLOY in the subject is a reduced lifespan, age-associated cardiomyopathy, reduced cardiac function, heart failure, increased fibrosis, optionally increased fibrosis of the myocardium, lung, and/or kidney; idiopathic pulmonary fibrosis (IPF); elevated left ventricular filling pressure (E/e′) indicative of diastolic dysfunction; and/or reduced cognitive function in the subject.

In some embodiments, the presently disclosed methods further comprise, consist essentially of, or consist of administering an anti-fibrotic therapy to the subject.

In some embodiments, the anti-fibrotic therapy comprises administering to the subject an effective amount of a small molecule anti-fibrotic, an anti-fibroblast antibody or a fragment thereof that binds to a polypeptide expressed by a fibroblast; a nucleic acid molecule that binds to and inhibits expression of a gene product expressed by a fibroblast in the subject; or any combination thereof.

In some embodiments, the small molecule anti-fibrotic is a withanolide compound (a group of naturally occurring steroids built on an ergostane skeleton), a fused ring derivative of 2-{[(2E)-3-(3,4-dimethoxyphenyl)prop-2-enoyl]amino}benzoic acid, tranilast (n-[3,4-dimethoxycinnamoyl]anthranilic acid), metabolites thereof, precursors thereof, or any combinations thereof.

In some embodiments, the anti-fibrotic therapy comprises an anti-fibroblast CAR-T cell therapy, and further wherein the anti-fibroblast CAR-T cell therapy employs CAR-T cells that are directed to fibroblast activation protein (FAP).

In some embodiments, the presently disclosed subject matter also relates to uses of inhibitors of TGFβ signaling for prevention and/or treatment of diseases, disorders, and/or conditions associated with mLOY. In some embodiments, the diseases, disorders, and/or conditions associated with mLOY are selected from the group consisting of age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and reduced cognitive function in a subject, as well as any combination thereof.

Accordingly, it is an object of the presently disclosed subject matter to provide compositions and methods for identifying subject at risk for diseases, disorders, and/or conditions associated with mosaic loss of chromosome Y in blood (mLOY). This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE FIGURES

FIGS. 1A-1I. Y chromosome deficiency in hematopoietic cells shortens life span and accelerates age-related cardiac dysfunction. FIG. 1A. Schematic of an exemplary approach for investigating mLOY in a mouse model. Lethally irradiated male C57BL6/J mice were reconstituted with hematopoietic stem cells transduced with lentivirus encoding Y chromosome targeting gRNA (LOY-gRNA, SEQ ID Nos. 1-4) or control gRNA (SEQ ID Nos. 5-6) and designated as mLOY and control mice, respectively. Phenotypic differences between mLOY and control mice during the natural aging process were analyzed. FIG. 1B. The efficiency of Y chromosome ablation analyzed by FISH. tRFP positive peripheral blood cells were collected from mice reconstituted with bone marrow cells transduced with lentivirus encoding a control gRNA (SEQ ID Nos. 5-6) or either of two different LOY-gRNAs (LOY-gRNA1,2; SEQ ID Nos. 1-4). The percentage of Y chromosome deficient (LOY) cells (light gray bars) and Y chromosome sufficient cells (XY; dark gray bars) in total blood cells are shown. Approximately 200 cells were analyzed for each condition. Unless otherwise indicated, all subsequent studies were performed with hematopoietic stem cells transduced with LOY-gRNA1. FIG. 1C. Representative images of FISH analysis of peripheral blood cells collected from mLOY and control mice. Green and red fluorescence indicate X and Y chromosomes, respectively. Arrows point out X and Y chromosomes, respectively. FIG. 1D. Karyotype analysis of LOY and control cells. Hematopoietic stem cells collected from mLOY and control mice were immortalized by lentivirus-mediated HoxB8 transduction and subjected to karyotype analysis. The arrow in the Control panel points to the Y chromosome, which is absent in the LOY panel. FIG. 1E. Bar graph of relative mRNA expression levels of genes on Y chromosome in RFP⁺ peripheral blood leukocytes in mLOY (black bars) and control (gray bars) mice (n=3 per group). Kdm5d: lysine demethylase 5D. Uty: ubiquitously transcribed tetratricopeptide repeat containing, Y-linked. Eif2s3y: eukaryotic translation initiation factor 2, subunit 3, structural gene Y-linked. Ddx3y: DEAD-box helicase 3 Y-linked. FIG. 1F. Kaplan-Meier survival curve for mLOY and control mice after bone marrow transplantation. X axis indicates time after bone marrow transplantation (day) (control n=37, mLOY n=38). FIG. 1G. Sequential echocardiographic analysis of mLOY and control mice after bone marrow transplantation at the indicated time points (month) (n=8-10 per group). FIG. 1H. Quantitative analysis of fibrotic area in heart section at 15-month after bone marrow transplantation (n=8-10 per group) (dark gray circles=mLOY; light gray circles=control). FIG. 1I. Flow cytometric analysis of fibroblast counts in heart tissue at 15-month after bone marrow transplantation (n=8-10 per group). The absolute numbers of cells were normalized by tissue weight. Dots in all panels represent individual samples (dark gray circles=mLOY; light gray circles=control). Data are shown as mean±SEM. Statistical analyses was performed using un-paired Student's t test (FIGS. 1E and 1H), Student's t test with Welch's correction (FIG. 1I) and log-rank test (FIG. 1F) and 2-way repeated measure ANOVA with Sidak's multiple comparison tests (FIG. 1G). LOY; loss of chromosome Y, mLOY; mosaic loss of chromosome Y, FS; fractional shortening, RFP; red fluorescent protein, Con; control. (*p<0.05, **p<0.01, ****p<0.0001). Error bars are mean±standard error of the mean (SEM).

FIGS. 2A-2C. Hematologic parameters of mLOY model. Lethally irradiated male C57BL6/J mice were reconstituted with hematopoietic stem cells transduced with lentivirus encoding Y chromosome targeting gRNA or control gRNA. FIG. 2A. Chimerism in mLOY model. Sequential flow cytometric analysis of peripheral blood leukocytes collected from mLOY and control mice at indicated time points. The frequency of donor cell chimerism is reflected by the percentage of RFP⁺ cells. No statistically significant differences were found by analysis of 2-way repeated measure ANOVA with Sidak's multiple comparison tests (dark gray circles=mLOY; light gray circles=control).

FIG. 2B. Peripheral blood chimerism in immune cell populations in blood of mLOY (dark gray circles) and control (light gray circles) mice. The percentage of RFP-positive cells were analyzed at 4 weeks after bone marrow transplantation using flow cytometry (n=6-7 per group). Statistical analysis was performed using un-paired Student's t test (Ly6C^{lo} Mono, CD4⁺, CD8⁺), Student's t test with Welch's correction (Neut, Ly6C^{hi} mono, B). Neut; neutrophils, Mono; monocytes, B; B cells, CD4⁺; CD4⁺ T cells, CD8⁺; CD8⁺ T cells. FIG. 2C. Mice with mLOY do not show hematologic abnormalities. The results of sequential hematological analysis of mLOY and control mice (n=8-10 per group). Data are shown as mean±SEM. Statistical analyses were performed using 2-way repeated measure ANOVA with Sidak's multiple comparison tests (dark gray circles=control & left bar light gray circles=mLOY & right bar; X axis indicates time after bone marrow transplantation (months).

FIGS. 3A-3F. Mice with mLOY display age-dependent cardiac phenotypes. FIG. 3A. Time-dependent changes in the echocardiographic parameters posterior wall diameter (PWd), left ventricular systolic diameter (LVDs), left ventricular diastolic diameter (LVDd), and Heart rate, in beats per minutes (bpm), in lightly anesthetized mice at the time of echocardiography. FIG. 3B. Heart weight (HW) relative to tibial length (TL) at 15 months after bone marrow transplantation (left). Time dependent change of body weight (BW) in mLOY (light gray circles; left bar in left panel) and control (dark gray circles; right bar in left panel) mice (n=8-10 per group). FIG. 3C. Representative images of cardiac fibrosis at 60 weeks after bone marrow transplantation in control mice (top panel) and mLOY mice (bottom panel). Scale bar: 0.1 mm. X axis indicates time after bone marrow transplantation (months). FIG. 3D. Representative pulse wave Doppler (top of left and center panels) and tissue Doppler (bottom of left and center panels) tracings, and ratio between mitral E wave and e′ wave (E/e′; bar graph in right panel) (n=3 per group). FIG. 3E. Systolic blood pressure (sBP), diastolic blood pressure (dBP) and mean BP in conscious mLOY (dark gray circles) and control (light gray circles) mice. Measurements were made on mice at 2 months post-BMT with non-invasive tail cuff blood pressure measurement system (n=8 per group). FIG. 3F. Serum levels of Renin 1 and Angiotensin II in mice at 4 months post-BMT in mLOY (dark gray circles) and control (light gray circles) mice. The measurement was done by ELISA. (n=6 per group). Statistical analyses were performed using 2-way repeated measure ANOVA with Sidak's multiple comparison tests (FIGS. 3A and 3B BW) and un-paired Student's t test (FIGS. 3B HW/TL, 3D, 3E, and 3F). *p<0.05, **p<0.01, ***p<0.005.

FIGS. 4A-4C. Organ fibrosis in mLOY mice. FIG. 4A. Pulmonary fibrosis analysis in mLOY (dark gray circles) and control (light gray circles) mice. Quantitative analysis of fibrotic area in lung section in mLOY and control mice at 60 weeks after bone marrow transplantation (n=8-10 per group). *p<0.05. FIG. 4B. Representative image and quantitative analysis of fibrotic area in lung sections at 4 weeks after bleomycin injection in mLOY and control mice (saline n=3, bleomycin n=7). Scale bar: m Statistical analyses were performed using Mann-Whitney U test (FIG. 4A) and 1-way ANOVA with Tukey's multiple comparison tests (FIG. 4B). FIG. 4C. Renal fibrosis analysis in aging mLOY (dark gray circles) and control (light gray circles) mice. Quantitative analysis of fibrotic area in kidney sections, and representative images, at 60 weeks after bone marrow transplantation in mLOY and control mice (n=8-10 per group). Statistical analyses were performed using un-paired Student's t test. Scale bar: 0.1 mm. **p<0.01, ***p<0.005, ****p<0.001. Bleo; bleomycin.

FIGS. 5A and 5B. Age-dependent cognitive decline in mLOY mice. FIG. 5A. Percentage spontaneous alternation in the Y-maze. FIG. 5B. percentage time spent with novel object from mLOY and control mice (n=3-8 per group). “Young” and “Old” mice were analyzed at 2 and 15 months after bone marrow transplantation, respectively. The cognitive function was assessed by two methods. Briefly, Y-maze test was used to evaluate spatial working memory. Mice were placed in the center of a Y-shaped maze (FIG. 5A, left) and allowed to freely explore the three arms, The number of arm entries and the number of triads were recorded in order to calculate the percentage of alternation. Percentage alternation was calculated as (number of alternations)/(number of arm entries−2)×100. Alternatively, novel object recognition test was used to evaluate recognition memory. Mice were familiarized to two identical objects placed in the arena as illustrated in the figure (FIG. 5B, left). After 10 min of interval away from the arena, mice were placed back but with one of the objects switched for a novel object. Time spent exploring each object was measured and percentage time spent with novel object calculated as follows: ((time exploring novel object)/(time exploring novel object+time exploring familiar object))×100. Statistical analyses were performed using un-paired Student's t test. *p<0.05. Dark gray circles: mLOY mice. Light gray circles: control mice. Error bars are mean±SEM.

FIGS. 6A-6H. Y chromosome deficiency in hematopoietic cells accelerates cardiac dysfunction in response to pressure overload. FIG. 6A. Schematic of experimental study for cardiac disfunction of mLOY mice in pressure overload model. mLOY mice were generated as described in previous section using LOY-gRNA1. At 4 weeks after bone marrow transplantation, mLOY mice or control mice were subjected to transverse aortic constriction (TAC) operation. FIG. 6B. Sequential echocardiographic analysis of mLOY and control mice after TAC operation at the indicated time points (control n=6, LOY n=7). FIG. 6C. Heart weight (HW) and lung weight (LW) relative to tibial length (TL) in sham and 4 weeks after TAC procedure (control sham n=6, control mLOY n=6, control TAC n=6, mLOY TAC n=7). FIG. 6D. Gene expression levels of heart failure markers (Nppa and b/a-MHC) in heart tissue in sham and 4-week after TAC operation (control sham n=6, control mLOY n=6, control TAC n=6, mLOY TAC n=7). FIG. 6E. Representative image and quantitative analysis (sham n=6, control mLOY n=6, control TAC n=6, mLOY TAC n=7) of fibrotic area in heart section in sham and 4-week after TAC operation. Scale bar: 100 m FIGS. 6F and 6G. Flow cytometric analysis of fibroblast (FIG. 6F) and endothelial cell (FIG. 6G) counts in heart tissue in sham and 4 weeks after TAC operation. Fibroblasts and endothelial cells are defined as CD45-CD31-MEF-SK4+ and CD45-CD31*, respectively. The absolute numbers of cells were normalized by tissue weight. (n=3-4 per group in sham, and n=12 per group in TAC mice). FIG. 6H. Mice with mLOY show comparable hypertrophic response of cardiac myocytes after TAC. Quantitative analysis of cross-sectional area of myocytes (CSA) in heart section at 4-week after TAC operation (n=6-7 per group). Dots in all panels represent individual samples. Data are shown as mean±SEM. Statistical analyses were performed using 2-way repeated measure ANOVA with Sidak's multiple comparison tests (FIG. 6B). 2-way ANOVA with Tukey's multiple comparison tests (FIGS. 6C-6G). mLOY; mosaic loss of chromosome Y, FS; fractional shortening. (*p<0.05, **p<0.01, ***p<0.001, ****p<0.0001). Dark gray circles: mLOY mice. Light gray circles: control mice. Error bars are mean±SEM.

FIGS. 7A-7C. Cardiac histology and echocardiographic parameters of TAC-treated mLOY and control mice. FIG. 7A. Sequential echocardiographic analysis of mLOY and control mice after TAC operation at the indicated time points (control n=6, LOY n=7). PWd, Posterior wall diameter; LVDs, Left ventricular end systolic diameter; LVDd, Left ventricular end diastolic diameter; HR, Heart rate in beats per minute (bpm). FIG. 7B. Fibrosis of the heart in sham and at 4 weeks after TAC operation is shown by Picro Sirius Red staining. Representative images of the fibrosis in the whole heart (top), left ventricle (middle) and perivascular area of coronary artery (bottom). The scale bar indicates 1000 m (top) and 100 m (middle and bottom). FIG. 7C. Representative images of the fibrosis in the left atrium of mLOY and control mice in sham and at 4 weeks after TAC operation, and the quantification of fibrosis is shown (control sham n=6, mLOY sham n=6, control TAC n=6, mLOY TAC n=7). Statistical analyses were performed using 2-way repeated measure ANOVA with Sidak's multiple comparison tests (FIGS. 7A and 7C). *p<0.05. ****: p<0.0001. Dark gray circles: mLOY mice. Light gray circles: control mice. Error bars are mean±SEM. X axis indicates time after bone marrow transplantation (Days).

FIGS. 8A-8F. Analysis of an independent CRISPR/Cas9-generated mLOY model. FIG. 8A. Efficiency of Y chromosome deletion in mLOY mice generated by LOY-gRNA2 and hematologic parameters in these mice. The percentage of RFP⁺ donor derived cells in peripheral blood of mLOY, generated with LOY-gRNA2, and control mice at 4 weeks after bone marrow transplantation. FIG. 8B. Hematological parameters in mLOY mice, generated with LOY-gRNA2, and control mice at 4 weeks after bone marrow transplantation. Data are shown as mean±SEM. Statistical analysis were performed using Mann-Whitney U test (WBC), two-tailed unpaired student's t test with Welch correction (Neut, Ly6C^{hi} mono) (FIG. 8A), two-tailed unpaired student's t test with Welch correction (WBC), Mann-Whitney U test (Hb), and two-tailed unpaired student t test (Plt) (FIG. 8B). FIG. 8C. Accelerated TAC-induced cardiac dysfunction in mLOY mice generated with LOY-gRNA2. Sequential echocardiographic analysis of mLOY (LOY-gRNA2) and control mice after TAC operation at the indicated time points (n=10 per group). At 4 weeks after bone marrow transplantation, mLOY (LOY-gRNA2) mice and control mice were subjected to transverse aortic constriction (TAC) operation. FS; fractional shortening, PWd; Posterior wall diameter, LVDs; Left ventricular end systolic diameter, LVDd; Left ventricular end diastolic diameter. FIG. 8D. Heart weight (HW) and lung weight (LW) relative to tibial length (TL) at 4 weeks after TAC. (n=8-9 per group). FIG. 8E. Serum levels of brain natriuretic peptide (BNP) at 4 weeks after TAC (n=10 per group). FIG. 8F. Flow cytometric analysis of fibroblast (left) and endothelial cell (right) counts in heart tissue at 4 weeks after TAC operation. Fibroblasts and endothelial cells were defined previously in FIG. 6F. 3. The absolute numbers of cells were normalized to tissue weight. (n=9-10 per group). Data are shown as mean±SEM. Statistical analysis were performed using 2-way repeated-measure ANOVA with post-hoc Sidak multiple-comparison tests (FIG. 8C), two-tailed unpaired student's t test (FIG. 8D), Mann-Whitney U test (FIG. 8E), two-tailed unpaired student's t test (fibroblast), and Mann-Whitney U test (endothelial cells) (FIG. 8F). *p<0.05, **p<0.01, ***p<0.005. Light gray circles left column: control mice; Dark gray circles, right column: mLOY mice. Error bars are mean±SEM.

FIGS. 9A-9E. Macrophage involvement in mLOY-mediated cardiac phenotype. FIG. 9A. Myocardial content of macrophage populations in control (light gray circles) and mLOY (dark gray circles) mice subjected to TAC. Flow cytometric analysis of cardiac macrophage populations at 1 week after TAC surgery. The absolute numbers of cells were normalized to tissue weight. (n=5 per group). Cardiac macrophages were defined as CD45⁺CD64⁺Ly6G⁻Ly6C^{low}. Statistical analyses were performed using two-tailed unpaired student's t test. *p<0.05. FIG. 9B. Cardiac parameters in control and myeloid cell-depleted mice. Sequential echocardiographic analysis of mLOY (dark gray circles) and control (light gray circles) mice after TAC at the indicated time points. At 4 weeks after bone marrow transplantation, mLOY mice or control mice were subjected to TAC. Anti-Gr-1 antibody or isotype control were intraperitoneally injected every 3 days for 4 weeks (n=5 per experimental group). FS: Fractional Shortening, PWd; Posterior wall diameter, LVDs; Left ventricular systolic diameter, LVDd; left ventricular diastolic diameter. FIG. 9C. Heart weight (HW) relative to tibial length (TL) at 4 weeks after the TAC procedure. FIG. 9D. Serum levels of brain natriuretic peptide (BNP) at 4 weeks after TAC surgery. FIG. 9E. Flow cytometric analysis of fibroblast (left) and endothelial cell (right) counts in heart tissue at 4 weeks after TAC. The absolute numbers of cells were normalized by tissue weight. (n=5 per group). Statistical analyses were performed using 2-way repeated measure ANOVA with Sidak's multiple comparison tests (FIG. 9A), 1-way ANOVA with Tukey's multiple comparison tests (FIGS. 9B-9D). *p<0.05, **p<0.01, ***p<0.005. (first bar—Control+isotype control, second bar—Control+anti-Gr1, third bar—mLOY+isotype control, fourth bar—mLOY+anti-Gr1).

FIGS. 10A-10E. Single cell RNA sequencing data from CD45*RFP⁺ cardiac cells at 7 days post-TAC. FIG. 10A. Plot shows UMAP dimensionality reduction with annotated clusters, separated by cells from control and mLOY samples, and expression of unique Y-chromosome genes separated by cells from control and mLOY samples. FIG. 10B. Dot plot of relative gene expression showing markers of annotated cell types, along with UMAP plots highlighting macrophage polarization markers. FIG. 10C. PHATE differentiation trajectory analysis of macrophagae clusters with identified and annotated clusters, and expression of genes identifying inflammatory (Il1b, Ccr2^{hi}) and fibrotic (Lyve1, Mrc1) macrophages. FIG. 10D. SCENIC analysis for transcription factor regulons that are significantly enriched in the control or mLOY cells within the inflammatory or fibrotic macrophage clusters. FIG. 10E. Expression of Tgfb1 within the inflammatory and fibrotic macrophage clusters. Uty: ubiquitously transcribed tetratricopeptide repeat containing, Y-linked. Kdm5d: lysine demethylase 5D. Eif2s3y: eukaryotic translation initiation factor 2, subunit 3, structural gene Y-linked. Ddx3y: DEAD-box helicase 3 Y-linked.

FIGS. 11A-11G. Inhibition of TGFβ1 reverses cardiac dysfunction in mLOY mice after TAC surgery. FIG. 11A. Single cell RNA sequencing from CD45+RFP+ cardiac cells 7 days post-TAC shown by UMAP dimensionality reduction with inflammatory and fibrotic macrophages and expression of Il1b and Lyve1 highlighted.

FIG. 11B. PHATE dimensionality reduction showing cells separated from control (light gray) or mLOY (dark gray) samples with non-activated, inflammatory, and fibrotic phenotypes labeled. FIG. 11C. Heatmap of transcription factor regulons within inflammatory or fibrotic macrophages related to Il1b or Tgfb1 expression, respectively, that was generated using SCENIC analysis of control and mLOY cells. Quantification of the relative percentage of control or mLOY cells contained in the Inflammatory or Fibrotic Macrophage clusters. FIG. 11D. Activation of TGFβ signaling in the heart was accessed by immunofluorescent staining of phosphorylated SMAD2 (pSMAD2). The number of pSMAD2 positive cells per view field and the percentage of pSMAD2 positive fibroblasts (right) is shown. Fibroblasts are defined as Vimentin positive cells. (n=6-7 fields per group). pSMAD2 positive and negative fibroblasts are indicated by green and orange arrows, respectively. Scale bar: 50 m. FIG. 11E. At 4 weeks after bone marrow transplantation, mLOY mice or control mice were subjected to TAC surgery. Anti-TGFβ antibody or isotype control were intraperitoneally injected every 3 days for 4 weeks. Sequential echocardiographic analysis of mLOY and control mice after TAC operation at the indicated time points (n=6-7 per group). FIG. 11F. Representative images and quantitative analysis of fibrotic area in heart section at 4-week after TAC procedure (n=6-7 per group). FIG. 11G. Flow cytometric analysis of fibroblast content in heart tissue at 4 weeks after TAC operation. The absolute numbers of cells were normalized by tissue weight. (n=6-7 per group). Dots in all panels represent individual samples. Data are shown as mean±SEM. Statistical analyses were performed using Chi-square test (FIG. 11B), Unpaired Student's t test (FIG. 11D), 2-way repeated measure ANOVA with Sidak's multiple comparison test (FIG. 11E), and 1-way ANOVA with Tukey's multiple comparison test (FIGS. 11F and 11G) FS; fractional shortening. (*p<0.05, **p<0.01, ***p<0.001, ****p<0.0001).

FIGS. 12A-12F. Cardiac macrophages from control and mLOY mice display differential gene expression. FIGS. 12A-12D. At 7 days post-TAC surgery, donor-derived blood neutrophils (Ly6G⁺RFP⁺ cells), cardiac neutrophils (CD45⁺Ly6G⁺RFP⁺ cells) and cardiac macrophages (CD45⁺Ly6G-CD64+RFP⁺ cells) were collected from mLOY and control mice using FACS, followed by ultra-low input RNA sequencing analysis. FIG. 12A. Principal component analysis of cardiac macrophages is shown along with representations of Y chromosome-expressed genes in cardiac macrophages in the mLOY and control conditions. FIG. 12B. Heatmaps of representative gene set enrichment analysis of TGFβ signaling and TGFβ binding in the bulk macrophage RNA to show statistically significant changes in expression. Principal component analysis of blood (FIG. 12C) and cardiac neutrophil (FIG. 12D) transcriptomes indicate limited differences between the mLOY and control conditions. Representations of Y chromosome-expressed genes in cardiac neutrophils. Kdm5d transcripts were not detectable. FIG. 12E. SCENIC analysis of neutrophil scRNAseq data is shown. Taf1 and Runx3 were the only significantly varied regulons in neutrophils, showing an increase in activity in the mLOY cells. Regulons identified by SCENIC analysis of the macrophage population (in FIGS. 10A-10E) did not display detectable differences between the mLOY and control conditions in the neutrophil population. FIG. 12F. SCENIC analysis of the monocyte population revealed changes in Klf2 and Jund regulons, but there was minimal impact on the regulons identified in the analysis of macrophage populations.

FIGS. 13A-13F. Role of TGFβ signaling in mLOY cardiac phenotype. Immunoblot analysis of TGFβ 1 (FIG. 13A) and SMAD2 phosphorylation (FIG. 13B) in heart tissue collected at 1 week after TAC operation. Activation of TGFβ signaling in the heart was accessed by immunoblot analysis of SMAD2 phosphorylation. Heart tissue at 1 week after the TAC procedure was homogenized and lysed. Equal amounts of protein were used for SDS-PAGE. The result of immunoblot (left) and quantification (right) are shown. (control sham n=1, mLOY sham n=1, control TAC n=3, mLOY TAC n=4). j-ACT; Beta-actin. FIGS. 13C-13F. At 4 weeks after bone marrow transplantation, mLOY mice or control mice were subjected to transverse aortic constriction (TAC) surgery. Anti-TGFβ antibody or isotype control were intraperitoneally injected every 3 days for 4 weeks. FIG. 13C. Sequential echocardiographic analysis of mLOY and control mice after TAC operation at the indicated time points. FIG. 13D. Heart weight (HW) and lung weight (LW) relative to tibial length (TL) at 4 weeks after TAC procedure (n=6-7 per group) (first bar—Control+isotype control, second bar—Control+anti-Gr1, third bar—mLOY+isotype control, fourth bar—mLOY+anti-Gr1). FIG. 13E. Serum levels of brain natriuretic peptide (BNP) at 4 weeks after the TAC procedure (n=6-7 per group)(first bar—Control+isotype control, second bar—Control+anti-Gr1, third bar—mLOY+isotype control, fourth bar—mLOY+anti-Gr1). FIG. 13F. Flow cytometric analysis of endothelial cell content in heart tissue at 4 weeks after TAC. The absolute numbers of cells were normalized by tissue weight. (n=6-7 per group). Statistical analysis was performed using 2-way repeated measure ANOVA with Sidak's multiple comparison tests. PWd, Posterior wall diameter; LVDs; left ventricular systolic diameter, LVDd; left ventricular end diastolic diameter. *p<0.05, ****p<0.001.(first bar—Control+isotype control, second bar—Control+anti-Gr1, third bar—mLOY+isotype control, fourth bar—mLOY+anti-Gr1).

FIG. 14. Y*^{X} model: An independent model of mLOY. Exemplary chromosome model showing Y chromosome replacement variant referred to as Y*^{X}. Control is the Y* variant that contains the Y*^{X} centromere and the full complement of the Y chromosome PAR region. (Y*=control; Y*^{X}=Loss of Y genes).

FIGS. 15A-15C. the Y*^{X} model of LOY leads to cardiac dysfunction. FIG. 15A. Pressure overload hypertrophy model showing Transverse Aortic Constriction (TAC). Aortic constriction to left and heart to right. FIG. 15 B. graph showing fractional shortening (FS, %). FIG. 15C. Graphs suggesting that the LOY effect in the TAC model may be due to loss of Y gene(s). **p<0.01. (Y*=control; Y*^{X}=Loss of Y genes).

FIG. 16. Four Y chromosome-encoded genes are appreciably expressed in mouse leukocytes. Y chromosome-encoded genes are void in the LOY condition.

FIGS. 17A-17H. CRISPR screening indicates only Uty loss affects cardiac function. FIG. 17A. Exemplary model TAC, a model of pressure overload cardiac hypertrophy. FIG. 17B. Graph showing fractional shortening associated with Eif2s3y. Ablation of Eif2s3y showed no detectable effects on cardiac dysfunction in the TAC model.

FIG. 17C. Graph showing fractional shortening associated with Ddx3y. Ablation of Ddx3y showed no detectable effects on cardiac dysfunction in the TAC model, however, the Ddx3y gene is severely downregulated when Uty is knocked out of the Y chromosome. FIG. 17 D. Graph showing fractional shortening associated with Kdm5d. Ablation of Kdm5d showed no detectable effects on cardiac dysfunction in the TAC model. FIG. 17E. Graph showing Fractional Shortening of CRISPR-mediated Uty ablation. FIG. 17F. Graph showing left ventricular posterior wall end-diastolic (LVPWTd) (millimeters) of CRISPR-mediated Uty ablation. FIG. 17G. Graph showing left ventricular internal diameter at end-sysolec (LVDs) of CRISPR-mediated Uty ablation. FIG. 17H. Graph showing left ventricular internal diameter at end-diastole (LVDd) of CRISPR-mediated Uty ablation. CRISPR-mediated Uty ablation revealed greater pathological cardiac remolding in response to TAC in FIGS. 17E-17H.

FIGS. 18A-18E. Bone Marrow Transplant (BMT) of Uty-deficient cells results in the same cardiac phenotype as the LOY mice, in the TAC model. FIG. 18A. Exemplary model of wild type (WT) or Uty-knockout (KO) bone marrow transplant mice in a TAC model. FIG. 18B. Uty express in bone marrow (BM) and peripheral blood (PB) cells, Y chromosome-encoded gene is void in the LOY condition. FIGS. 18C-18E. Graphs show that Uty as a gene target on the Y chromosome confers, at least in part, the effect of mLOY on cardiac pathology. Here, FS=fractional shortening, HW=heart weight and TL=Tibia length. *p<0.05, **p<0.01, ***p<0.005, ****p<0.0001.

FIG. 19. Kidney fibrosis detected in aging mLOY mouse model. Quantitative analysis of fibrotic area in kidney sections, and representative images, at 60 weeks after bone marrow transplantation in mLOY and control mice (n=8-10 per group). Statistical analyses were performed using un-paired Student's t test. Scale bar: 0.1 mm. ***p<0.005.

FIGS. 20A and 20B. Renal dysfunction in aging mLOY (CRISPR/Cas9) mice. FIG. 20A. Exemplary model of CRISPR-mediated LOY. FIG. 20B. mLOY mice show increased Blood Urea Nitrogen (BUN) levels, a biomarker of kidney dysfunction.

FIGS. 21A and 21B. Renal dysfunction in 15-month-old hematopoietic Uty-KO mice. FIG. 21A. Exemplary model of Uty-KO mice. FIG. 21B. Aged hematopoietic mosaic UTY^{-/-} mice display higher BUN levels

FIG. 22. Aristolochic Acid (AA)-induced Chronic Kidney Disease (CKD) model.

FIGS. 23A and 23B. AA CKD model with hematopoietic Uty-KO. FIG. 23A Exemplary model of AA Chronic Kidney Disease. FIG. 23B. Hematopoietic Uty^{-/-} mice show more severe renal dysfunction (Blood Urea Nitrogen).

FIGS. 24A-24E. AA model of CKD: elevated renal fibrosis with hematopoietic Uty-KO. FIG. 24A. Exemplary model of AA Chronic Kidney Disease. FIGS. 24B-24E. Graphs and staining show hematopoietic Uty^{-/-} mice display more severe renal fibrosis.

FIGS. 25A-25C. Kidneys of aging mLOY mice (CRISPR/Cas9) display elevated markers of cellular senescence. FIG. 25A. Exemplary model of CRISPR-mediated LOY. FIG. 25B. Kidneys of mLOY mice showed increased p16 & p21 expression. FIG. 25C. SA-3-gal staining showed positive in mLOY mice.

FIGS. 26A-26C. Senescent kidneys in aging hematopoietic Uty-KO mice. FIG. 26A. Exemplary model of wild type (WT) or Uty-knockout (KO) aged kidneys. FIGS. 26B and 26C. Kidneys in hematopoietic UTY−/− mice displayed increased expression of the Sen Mayo diagnostic gene set.

FIGS. 27A and 27B. The AA CKD model with hematopoietic Uty-KO (Analysis of cellular senescence markers). FIG. 27A. Exemplary model of AA Chronic Kidney Disease. FIG. 27B. Hematopoietic Uty^{-/-} mice showed a higher expression of senescence-marker genes in kidneys in the AA CKD model.

FIGS. 28A-28C. The effect of the senolytic ABT-263 on lifespan and kidney function in aging hematopoietic Uty-KO mice. FIG. 28A. Exemplary model of ABT-263 mice. FIGS. 28B and 28C. ABT-263 promoted survival and suppressed the progression of renal dysfunction in aging mice.

### DETAILED DESCRIPTION

Hematopoietic mosaic loss of Y chromosome (mLOY) is associated with increased risk of mortality and a variety of age-related diseases in males, but causal and mechanistic relationships have yet to be established. Here it is shown that mice reconstituted with bone marrow cells lacking the Y chromosome display increased mortality and age-related profibrotic pathologies including a progressive decline in cardiac function. Accelerated cardiac dysfunction and elevated fibrosis was also observed in younger mice subjected to a pressure-overload model of heart failure. Bone marrow-derived, cardiac macrophages lacking the Y chromosome exhibited polarization toward a more fibrotic and less inflammatory phenotype. Treatment with a TGFβ neutralizing antibody led to greater amelioration of heart failure in mice reconstituted with mLOY compared to wild-type bone marrow. Together, the presently disclosed results indicated that hematopoietic mLOY was causally linked to heart failure in males.

Additionally, provided herein are clinical data that show that loss of the Y chromosome (LOY) in men contributes to idiopathic pulmonary fibrosis (IPF). Also disclosed herein is the identification of Uty gene on the Y chromosome as being a regulator of the LOY disease phenotype. The presently disclosed subject matter was evaluated using independent methodologies (Y*^{X} and Uty-ablation/knock-out). The presently disclosed subject matter provides data implicating mLOY in chronic kidney disease and studies indicating that men with mLOY can exhibit a better response to anti-fibrotic drugs. The presently disclosed subject matter extends these observations to show that men with mLOY can exhibit responses to senolytic drugs.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about.” The term “about”, as used herein when referring to a measurable value such as an amount of mass, weight, time, volume, concentration, or percentage, is meant to encompass variations of in some embodiments ±20%, in some embodiments ±10%, in some embodiments±5%, in some embodiments ±1%, in some embodiments ±0.5%, and in some embodiments±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent,” as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising,” “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell,” refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in Table 1:

TABLE 1
Amino Acid Codes and Functionally Equivalent Codons

| | 3-Letter | 1-Letter | Functionally |
|---|---|---|---|
| Full Name | Code | Code | Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; |
| | | | CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; |
| | | | UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; |
| | | | CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the compositions of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the compositions of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

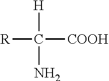

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fabi monomer. The Fabi monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain,” as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA or short hairpin RNA (shRNA).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible,” as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample,” as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound,” as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in Table 2:

TABLE 2
Exemplary Conservative Amino Acid Substitutions

| Group | Characteristics | Amino Acids |
|---|---|---|
| A. | Small aliphatic, nonpolar, or | Ala, Ser, Thr, Pro, Gly |
| | slightly polar residues | |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes, or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition,” “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment,” “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin & Altschul, 1990a, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated,” when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder.

A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex.

Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample,” as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

As used herein, the term FOXO4 related peptide refers to Forkhead box protein OA that is encoded by the FOXO4 gene. In some embodiments, the FOXO4 related peptide is a human peptide. In some embodiments, the FOXO4 related peptide is a synthetic peptide. The synthetic FOXO4 peptide includes but is not limited to FOXO4-D-Retro-Inverso (DRI) peptide and FOXO4-TP53 peptide.

As used herein, the term BCL-2 inhibitor refers to a selective inhibitor of the anti-apoptotic protein B-cell lymphoma 2 (BCL-2). In some embodiments, the BCL-2 inhibitor includes but is not limited to bcl-2 antisense oligodeoxynucleotide G3139, 4-{4-[(4′-Chloro[1,1′-biphenyl]-2-yl)methyl]piperazin-1-yl}-N-(4-{[(2R)-4-(dimethylamino)-1-(phenylsulfanyl)butan-2-yl]amino}-3-nitrobenzene-1-sulfonyl)benzamide, 4-(4-{[2-(4-Chlorophenyl)-5,5-dimethyl-1-cyclohexen-1-yl]methyl}-1-piperazinyl)-N-[(4-{[(2R)-4-(4-morpholinyl)-1-(phenylsulfanyl)-2-butanyl]amino}-3-[(trifluoromethyl)sulfonyl]phenyl)sulfonyl]benzamide and 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]45yridine-5-yloxy)benzamide.

As used herein, the term Src inhibitor refers to a class of inhibitors that target the Src Kinase family of tyrosine kinase, which is transcribed by the Src proto-oncogene. In some embodiments, Src inhibitor includes but is not limited to N-benzyl-2-(5-(4-(2-morpholinoethoxy)phenyl)pyridin-2-yl)acetamide, 4-(2,4-dichloro-5-methoxyanilino)-6-methoxy-7-[3-(4-methylpiperazin-1-yl)propoxy]quinoline-3-carbonitrile, N-(5-chloro-1,3-benzodioxol-4-yl)-7-[2-(4-methylpiperazin-1-yl)ethoxy]-5-(oxan-4-yloxy)quinazolin-4-amine, 1-tert-butyl-3-(naphthalen-1-ylmethyl)-1H-pyrazolo[3,4-d]pyrimidin-4-amine, 1-tert-Butyl-3-(4-chlorophenyl)-1H-pyrazolo[3,4-d]pyrimidin-4-amine and N-(2-chloro-6-methylphenyl)-2-({6-[4-(2-hydroxyethyl)piperazin-1-yl]-2-methylpyrimidin-4-yl}amino)-1,3-thiazole-5-carboxamide.

As used herein, the term USP7 inhibitor refers to a class of inhibitors that increase p53 levels, induce cell cycle arrest, and ultimately cause cell death in cellular and animal models. In some embodiments, the USP7 inhibitor includes but is not limited to small molecule inhibitors of the USP7.

As used herein, the terms anti-fibrotic agent and anti-fibrotic therapy are used interchangeably. Anti-fibrotic agents refer to a class of drugs for the treatment of idiopathic pulmonary fibrosis (IPF), systemic sclerosis-associated interstitial lung disease and non-small cell lung cancer. In some embodiments, the anti-fibrotic agent includes but is not limited to 5-methyl-1-phenyl-1,2-dihydropyridin-2-one and methyl (3Z)-3-[({4-[N-methyl-2-(4-methylpiperazin-1-yl)acetamido]phenyl}amino)(phenyl)methylidene]-2-oxo-2,3-dihydro-1H-indole-6-carboxylate.

As used herein, the term “UTY” refers to the ubiquitously transcribed tetratricopeptide repeat containing, Y-linked (UTY) genetic locus as well as all gene products derived therefrom, including nucleic acid gene products and polypeptide gene products. Exemplary biosequences that are derived from the UTY include human biosequences including, but not limited to Accession Number NM_182660.1 (Homo sapiens ubiquitously transcribed tetratricopeptide repeat containing, Y-linked (UTY), transcript variant 1, mRNA) of the GENBANK® biosequence database, which is set forth herein as SEQ ID NO: 16. SEQ ID NO: 16 encodes a polypeptide with the amino acid sequence disclosed as Accession No. NP_872601.1, which is SEQ ID NO: 17.

It is noted that the human UTY genetic locus encodes at least 30 different transcript variants, all of which are understood to be encompassed by the presently disclosed subject matter. Thus, the inclusion of SEQ ID NOs: 16 and 17 is understood to be representative only, and any and all UTY genetic loci and their gene products are encompassed by the term “UTY”. These include other transcript variants/isoforms such as the murine orthologs represented by Accession No. NM_009484.3 of the GENBANK® biosequence database (Mus musculus ubiquitously transcribed tetratricopeptide repeat containing, Y-linked (Uty), transcript variant 1, mRNA), which is SEQ ID NO: 18 and which encodes Accession No. NP_033510.2 (histone demethylase UTY isoform 1 [Mus musculus]), which is SEQ ID NO: 19.

As used herein, the term “Eif2s3y” refers to the eukaryotic translation initiation factor 2, subunit 3, structural gene Y-linked (Eif2s3y) genetic locus as well as all gene products derived therefrom, including nucleic acid gene products and polypeptide gene products. Exemplary biosequences that are derived from the Eif2s3y locus include human and mouse biosequences including, but not limited to Accession Number NM_012011.2 (SEQ ID NO: 20; eukaryotic translation initiation factor 2 subunit 3, Y-linked isoform 1 [Mus musculus]) of the GENBANK® biosequence database. SEQ ID NO: 20 encodes a polypeptide with the amino acid sequence disclosed as Accession No. NP_036141.1, which is SEQ ID NO: 21.

It is noted that the human and mouse Eif2s3y genetic loci also encode several different transcript variants, all of which are understood to be encompassed by the presently disclosed subject matter. Thus, the inclusion of SEQ ID NOs: 20 and 21 is understood to be representative only, and any and all Eif2s3y genetic loci and their gene products are encompassed by the term “Eif2s3y”. These include other murine transcript variants/isoforms and other vertebrate (in some embodiments, mammalian, and in some embodiments human) orthologs and variants.

As used herein, the term “Ddx3y” refers to the DEAD box helicase 3, Y-linked (Ddx3y) genetic locus as well as all gene products derived therefrom, including nucleic acid gene products and polypeptide gene products. Exemplary biosequences that are derived from the Ddx3y locus include human and mouse biosequences including, but not limited to Accession Number NM_012008.2 (SEQ ID NO: 22; Mus musculus DEAD box helicase 3, Y-linked (Ddx3y), mRNA) of the GENBANK® biosequence database. SEQ ID NO: 22 encodes a polypeptide with the amino acid sequence disclosed as Accession No. NP_036138.1, which is SEQ ID NO: 23. Thus, the inclusion of SEQ ID NOs: 22 and 23 is understood to be representative only, and any and all Ddx3y genetic loci and their gene products are encompassed by the term “Ddx3y”. These include other murine transcript variants/isoforms and other vertebrate (in some embodiments, mammalian, and in some embodiments human) orthologs and variants.

As used herein, the term “Kdm5d” refers to the lysine (K)-specific demethylase 5D (Kdm5d) genetic locus as well as all gene products derived therefrom, including nucleic acid gene products and polypeptide gene products. Exemplary biosequences that are derived from the Kdm5d locus include human and mouse biosequences including, but not limited to Accession Number NM_011419.3 (SEQ ID NO: 24; Mus musculus lysine (K)-specific demethylase 5D (Kdm5d), mRNA) of the GENBANK® biosequence database. SEQ ID NO: 24 encodes a polypeptide with the amino acid sequence disclosed as Accession No. NP_035549.1, which is SEQ ID NO: 25. Thus, the inclusion of SEQ ID NOs: 24 and 25 is understood to be representative only, and any and all Kdm5d genetic loci and their gene products are encompassed by the term “Kdm5d”. These include other murine transcript variants/isoforms and other vertebrate (in some embodiments, mammalian, and in some embodiments human) orthologs and variants.

As used herein, the term “TGFB1” refers to the transforming growth factor beta 1 (TGFB1) genetic locus as well as all gene products derived therefrom, including nucleic acid gene products and polypeptide gene products. Exemplary biosequences that are derived from the TGFB1 include human biosequences including, but not limited to Accession Number NM_000660.7 (Homo sapiens transforming growth factor beta 1 (TGFB1), mRNA) of the GENBANK® biosequence database, which is set forth herein as SEQ ID NO: 26. SEQ ID NO: 26 encodes a polypeptide with the amino acid sequence disclosed as Accession No. NP_000651.3, which is SEQ ID NO: 27. Thus, the inclusion of SEQ ID NOs: 12 and 27 is understood to be representative only, and any and all TGFB1 genetic loci and their gene products are encompassed by the term “TGFB1”. These include other transcript variants/isoforms such as the murine orthologs represented by Accession No. NM_011577.2 of the GENBANK® biosequence database, which is SEQ ID NO: 28 and which encodes Accession No. NP_035707.1, which is SEQ ID NO: 29.

As used herein, anti-fibroblast antibody refers to an antibody that detects fibroblasts which are a type of cell that contributes to the formation of connective tissue.

Chimeric antigen receptors (CARs) are artificially constructed hybrid proteins or polypeptides containing the antigen binding domains of an antibody (such as, but not limited to an scFv) linked to one or more T-cell signaling domains. Characteristics of CARs include their ability to redirect T-cell specificity and reactivity toward a selected target in a non-MHC-restricted manner, thereby exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. CARs and methods to prepare the same are described generally in U.S. Pat. Nos. 6,410,319; 8,389,282; and 10,059,923; as well as U.S. Patent Application Publication Nos. 2007/0036773, 2009/0180989, 2009/0257991, 2011/0038836, 2012/0058051, 2012/0213783, and 2012/0252742, each of which is incorporated herein by reference in its entirety.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Exemplary Embodiments

In some embodiments, the presently disclosed subject matter relates to methods for identifying subjects at risk for reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function. In some embodiments, the method comprises determining if the subject has mosaic loss of chromosome Y in blood (mLOY), wherein the presence of mLOY in the subject is indicative of the subject being at risk for reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function.

As disclosed herein, it has been determined that mLOY in blood of a subject is indicative of various diseases, disorders, and/or conditions, including but not limited to reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and reduced cognitive function. As such, in some embodiments white blood cells or macrophages isolated from the subject, optionally from the subject's blood or heart, are screened for the mLOY. In some embodiments, TGFβ 1 gene expression is determined in the subject. In some embodiments, downstream consequences of increased TGFβ1 gene expression are identified. In some embodiments, an effective amount of an inhibitor of TGFβ signaling is administered via a route and in an amount sufficient to inhibit TGFβ signaling in the subject.

While not wishing to be bound by theory, mosaic loss of chromosome Y and the Uty gene in blood cells can result in senescent cell accumulation, contributing to tissue dysfunction and biological aging. In some embodiments, hematopoietic Uty^{-/-} results in higher expression of senescence-marker genes in kidneys. Given that mLOY in blood of a subject can be indicative of certain diseases, disorders, and/or conditions, in some embodiments the presently disclosed subject matter relates to methods for treating and/or preventing diseases, disorders, and/or conditions associated with mLOY in subjects. In some embodiments, idiopathic pulmonary fibrosis (IPF) is treated in the subject, In some embodiments, the methods comprise, consist essentially of, or consist of administering to subject with mLOY an inhibitor of TGFβ signaling via a route and in an amount sufficient to inhibit TGFβ signaling in the subject and/or administering an effective amount of a senolytic agent to the subject, whereby a disease, disorder, and/or condition associated with mLOY in the subject is treated.

Various inhibitors of TGFβ signaling are known, and include anti-TGFβ antibodies (and/or fragments thereof that binds to TGFβ polypeptides to inhibit TGFβ signaling in the subject). In some embodiments, the TGFβ inhibitor comprises pirfenidone. Other inhibitors include nucleic acid molecules that bind to an inhibit expression of a TGFβ gene product in the subject. The presently disclosed subject matter thus encompasses the use of all types of inhibitors of the pathways described herein. The inhibitors include, but are not limited to, oligonucleotides, antisense oligonucleotide, nucleic acid, siRNA, shRNA, an antibody, antibody fragment, humanized antibody, monoclonal antibody, fragments thereof, aptamer, phylomer, protein, and small molecules such as drugs, optionally pirfenidone.

In some embodiments, the small molecule is a small molecule anti-fibrotic. In some embodiment, the small molecule comprises a withanolide compound, a fused ring derivative of 2-{[(2E)-3-(3,4-dimethoxyphenyl)prop-2-enoyl]amino}benzoic acid, tranilast (n-[3,4-dimethoxycinnamoyl]anthranilic acid), metabolites thereof, precursors thereof, or any combinations thereof.

Accordingly, in some embodiments the presently disclosed subject matter relates to uses of inhibitors of TGFβ signaling and/or a senolytic agent for prevention and/or treatment of age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function in a subject.

In some embodiments, an effective amount of the senolytic agent is administered to the subject. In some embodiments, an effective amount of a senolytic agent is administered to the subject, whereby a disease, disorder, and/or condition associated with mosaic loss of chromosome Y in blood (mLOY) in the subject is treated. In some embodiments the senolytic comprises a FOXO4-related peptide, a BCL-2 inhibitor, a Src inhibitor, a USP7 inhibitor, N-(2-chloro-6-methylphenyl)-2-[[6-[4-(2-hydroxyethyl)-1-piperazinyl]-2-methyl-4-pyrimidinyl]amino]-5-thiazole carboxamide monohydrate and 2-(3,4-dihydroxyphenyl)-3,5,7-trihydroxychromen-4-one, Fisetin (3,3′,4′,7-tetrahydroxyflavone), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-trifluoromethanesulfonyl)benzene-1-sulfonyl)benzamide, azithromycin and roxithromycin, a senescence-specific killing compound 1, (2R,3S,4S,5R,6S)-2-(acetoxymethyl)-6-(4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)-2-nitrophenoxy)tetrahydro-2H-pyran-3,4,5-triyl triacetate, a BIRC5 inhibitor, a glutaminase-1 (GLS1) inhibitor, an anti-Glycoprotein Nmb (GPNMB) vaccine, a cardiac glycoside, 25-hydroxycholesterol (25HC), (2R,3R,4S)-2-(3,4-dihydroxyphenyl)-4-[(2R,3R)-2-(3,4-dihydroxyphenyl)-3,5,7-trihydroxy-3,4-dihydro-2H-chromen-8-yl]-8-[(2R,3R,4R)-2-(3,4-dihydroxyphenyl)-3,5,7-trihydroxy-3,4-dihydro-2H-chromen-4-yl]-3,4-dihydro-2H-chromene-3,5,7-triol, (3E,5E)-3,5-bis[(2-fluorophenyl)methylidene]piperidin-4-one, a heat shock protein 90 (HSP90) inhibitor, and any combination thereof.

In some embodiments the senolytic agent is selected from the group consisting of a FOXO4-related peptide, a BCL-2 inhibitor, a Src inhibitor, a USP7 inhibitor, N-(2-chloro-6-methylphenyl)-2-[[6-[4-(2-hydroxyethyl)-1-piperazinyl]-2-methyl-4-pyrimidinyl]amino]-5-thiazole carboxamide monohydrate and 2-(3,4-dihydroxyphenyl)-3,5,7-trihydroxychromen-4-one, Fisetin (3,3′,4′,7-tetrahydroxyflavone), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-trifluoromethanesulfonyl)benzene-1-sulfonyl)benzamide, azithromycin and roxithromycin, a senescence-specific killing compound 1, (2R,3S,4S,5R,6S)-2-(acetoxymethyl)-6-(4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)-2-nitrophenoxy)tetrahydro-2H-pyran-3,4,5-triyl triacetate, a BIRC5 inhibitor, a glutaminase-1 (GLS1) inhibitor, an anti-Glycoprotein Nmb (GPNMB) vaccine, a cardiac glycoside, 25-hydroxycholesterol (25HC), (2R,3R,4S)-2-(3,4-dihydroxyphenyl)-4-[(2R,3R)-2-(3,4-dihydroxyphenyl)-3,5,7-trihydroxy-3,4-dihydro-2H-chromen-8-yl]-8-[(2R,3R,4R)-2-(3,4-dihydroxyphenyl)-3,5,7-trihydroxy-3,4-dihydro-2H-chromen-4-yl]-3,4-dihydro-2H-chromene-3,5,7-triol, (3E,5E)-3,5-bis[(2-fluorophenyl)methylidene]piperidin-4-one, a heat shock protein 90 (HSP90) inhibitor, and any combination thereof.

In some embodiments, the anti-fibrotic agent is a treatment for idiopathic pulmonary fibrosis (IPF). In some embodiments, the anti-fibrotic agent comprises nintedanib, pirfenidone and any combination thereof. In some embodiments, the anti-fibrotic agent is selected from the group consisting of nintedanib, pirfenidone, and any combination thereof. In some embodiments, the small molecule anti-fibrotic is a withanolide compound, a fused ring derivative of 2-{[(2E)-3-(3,4-dimethoxyphenyl)prop-2-enoyl]amino}benzoic acid, tranilast (n-[3,4-dimethoxycinnamoyl]anthranilic acid), metabolites thereof, precursors thereof, or any combinations thereof. In some embodiments, the anti-fibrotic therapy comprises an anti-fibroblast CAR-T cell therapy, and further wherein the anti-fibroblast CAR-T cell therapy employs CAR-T cells that are directed to fibroblast activation protein (FAP).

As used herein, the phrase “antigen-specific targeting region” (ASTR) refers to the region of a CAR that targets (i.e., binds to) specific antigens and/or epitopes. The CARs of the presently disclosed subject matter comprise in some embodiments one ASTR (i.e., are monospecific) and in some embodiments comprise two targeting regions which target two different antigens and/or epitopes (i.e., are bispecific). In some embodiments, CARs comprise three or more targeting regions which target at least three or more different antigens (i.e., are trispecific or multispecific). The targeting regions on the CAR are extracellular. In some embodiments, the antigen-specific targeting regions comprise an antibody or a functional equivalent thereof or a fragment thereof or a derivative thereof, and in some embodiments each of the targeting regions targets a different antigen or epitope. The targeting regions can comprise full length heavy chain, Fab fragments, single chain Fᵥ (scFv) fragments, divalent single chain antibodies or diabodies, each of which are specific to the target antigen. There are, however, numerous alternatives, such as linked cytokines (which leads to recognition of cells bearing the cytokine receptor), affibodies, ligand binding domains from naturally occurring receptors, soluble protein/peptide ligand for a receptor (for example on a tumor cell), peptides, and vaccines to prompt an immune response, which may each be used in various embodiments of the presently disclosed subject matter. In fact, almost any molecule that binds a given antigen with high affinity can be used as an ASTR, as will be appreciated by those of skill in the art.

Thus, as used herein, the terms “Chimeric Antigen Receptor”, “CAR”, and “CARs” refer to engineered receptors, which graft an antigen specificity onto cells (for example T cells such as naive T cells, central memory T cells, effector memory T cells, or combination thereof, which in some embodiments are referred to herein as “CAR-T cells”). CARs are also known as artificial T-cell receptors, chimeric T-cell receptors, or chimeric immunoreceptors. The CARs of the presently disclosed subject matter comprise one or more ASTRs, an extracellular domain, a transmembrane domain, one or more co-stimulatory domains, and an intracellular signaling domain. In those embodiments where two or more ASTRs are present, the two or more ASTRs can target at least two different antigens and can be arranged in tandem and separated by linker sequences. In some embodiments, the extracellular spacer domain is optional. In some embodiments, the CAR is a monospecific CAR that targets a fibroblast activation protein (FAP) antigen or epitope.

As used herein, the phrase “co-stimulatory domain” (CSD) refers to the portion of the CAR that enhances the proliferation, survival, and/or development of memory cells. The CARs of the presently disclosed subject matter can comprise one or more co-stimulatory domains. In some embodiments, each co-stimulatory domain comprises the costimulatory domain of one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40, or any combinations thereof. Other co-stimulatory domains (e.g., from other proteins) will be apparent to those of skill in the art and can be used in connection with alternate embodiments of the presently disclosed subject matter.

As used herein, the phrase “extracellular spacer domain” (ESD) refers to the hydrophilic region that is between the ASTR and the transmembrane domain. In some embodiments, the CARs of the presently disclosed subject matter comprise an extracellular spacer domain. In some embodiments, the CARs of the presently disclosed subject matter do not comprise an extracellular spacer domain. The extracellular spacer domains can include, but are not limited to, Fc fragments of antibodies or fragments or derivatives thereof, hinge regions of antibodies or fragments or derivatives thereof, CH2 regions of antibodies, CH3 regions of antibodies, artificial spacer sequences, or combinations thereof. Examples of extracellular spacer domains include, but are not limited to, CD8α hinge, and artificial spacers made of polypeptides which can be as small as, for example, Gly3 or CH1 and CH3 domains of IgGs (such as but not limited to human IgG4). In some embodiments, the extracellular spacer domain is any one or more of (i) a hinge, CH2, and CH3 regions of IgG4; (ii) a hinge region of IgG4; (iii) a hinge and CH2 of IgG4; (iv) a hinge region of CD8a; (v) a hinge, CH2, and CH3 regions of IgG1; (vi) a hinge region of IgG1; (vi) a hinge and CH2 region of IgG1; and/or (vii) a hinge region of IgD. Other extracellular spacer domains will be apparent to those of skill in the art and may be used in connection with any embodiments of the presently disclosed subject matter.

In some embodiments, the binding domain of a CAR of the presently disclosed subject matter is followed by a hinge region, which refers to the region that moves the antigen binding domain away from the effector cell surface to enable proper cell/cell contact, antigen binding, and activation (see e.g., Patel et al., 1999). In some embodiments, a hinge region is an immunoglobulin hinge region and can be a wild type immunoglobulin hinge region or a modified immunoglobulin hinge region. Other exemplary hinge regions used in the CARs described herein include the hinge region derived from the extracellular regions of type 1 membrane proteins such as CD8a, CD4, CD28, and CD7, which can be wild type hinge regions from these molecules or can be modified. In some embodiments, a hinge region is an IgD hinge region.

As used herein, the phrase “modified hinge region” refers to (a) a wild type hinge region with in some embodiments up to 30% amino acid changes (e.g., up to 25% amino acid changes, up to 20% amino acid changes, up to 15% amino acid changes, up to 10% amino acid changes, or up to 5% amino acid changes, including but not limited to amino acid substitutions, additions, and/or deletions); (b) a portion of a wild type hinge region that is in some embodiments at least 10 amino acids in length (e.g., at least 11, 12, 13, 14, 15, 16, 17, 18, 19, 20, 25, 30, 35, 40, 45, 50, or more amino acids) in length with in some embodiments up to 30% amino acid changes (e.g., up to 25% amino acid changes, up to 20% amino acid changes, up to 15% amino acid changes, up to 10% amino acid changes, or up to 5% amino acid changes, including but not limited to amino acid substitutions, additions, and/or deletions); or (c) a portion of a wild type hinge region that comprises the core hinge region (which in some embodiments can be 4, 5, 6, 7, 8, 9, 10, 11, 12, 13, 14, or 15, or at least 4, 5, 6, 7, 8, 9, 10, 11, 12, 13, 14, or 15 amino acids in length). When a modified hinge region is interposed between and connecting a binding domain and another region (e.g., a transmembrane domain) in the CARs described herein, it allows the chimeric fusion protein to maintain specific binding to its target (e.g., tMUC).

As used herein, the phrase “intracellular signaling domain” (ISD) or “cytoplasmic domain” refer to the portion of the CAR which transduces the effector function signal and directs the cell to perform its specialized function. Examples of domains that transduce the effector function signal include but are not limited to the zeta chain of the T-cell receptor complex or any of its homologs (e.g., the eta chain, FcR1 γ and β chains, MB1 (Igα) chain, B29 (Igβ) chain, etc.), human CD3 zeta chain, CD3 polypeptides (delta and epsilon), syk family tyrosine kinases (Syk, ZAP 70, etc.), src family tyrosine kinases (Lck, Fyn, Lyn, etc.), and other molecules involved in T-cell transduction, such as CD2, CD5, and CD28. Other intracellular signaling domains will be apparent to those of skill in the art and can be used in connection with any embodiments of the presently disclosed subject matter.

As used herein, the phrases “linker”, “linker domain”, and “linker region” refer to an oligo- or polypeptide region from about 1 to 100 amino acids in length, which links together any of the domains and/or regions of a CAR of the presently disclosed subject matter. In some embodiments, linkers comprise, consist essentially of, or consist of flexible residues like glycine and serine so that the adjacent protein domains are free to move relative to one another. Longer linkers can be used when it is desirable to ensure that two adjacent domains do not sterically interfere with one another, such as can be the case with bispecific, trispecific, and multispecific CARs. Linkers can be cleavable or non-cleavable. Examples of cleavable linkers include 2A linkers (for example T2A; see U.S. Pat. No. 8,802,374 to Jenson, incorporated herein by reference in its entirety), 2A-like linkers, or functional equivalents thereof, and combinations thereof. In some embodiments, the linkers include the picornaviral 2A-like linker, cis-acting hydrolase element (CHYSEL) sequences of porcine teschovirus (P2A), Thosea asigna virus (T2A), or combinations, variants, and functional equivalents thereof. In some embodiments, the linker sequences can comprise Asp-Val/Ile-Glu-X-Asn-Pro-Gly^{2A}-Pro^{2B} motif, which results in cleavage between the 2A glycine and the 2B proline. Other linkers will be apparent to those of skill in the art and may be used in connection with any embodiments of the presently disclosed subject matter.

As used herein, the phrase “transmembrane domain” (TMD or TD) refers to the region of the CAR that crosses the plasma membrane. The transmembrane domains of the CARs of the presently disclosed subject matter are the transmembrane regions of a transmembrane protein (for example Type I transmembrane proteins), an artificial hydrophobic sequence, or a combination thereof. Other transmembrane domains will be apparent to those of skill in the art and can be used in connection with any embodiments of the presently disclosed subject matter.

CARs and the T cells that have been modified to express CARs can be described as being “first generation”, “second generation”, “third generation”, or “fourth generation” based on the various components that are present in the CARs. “First generation” CARs include an antigen binding domain, transmembrane domain, and an intracellular domain, typically a CD3zeta intracellular domain. “Second generation” CARs further comprise a costimulatory domain. “Third generation” CARs further comprise other signaling domains, such as but not limited to 4-IBB signaling domains and/or OX40 signaling domains. “Fourth generation” CAR T cells typically are characterized by the presence of a second or third generation CAR, and have been further modified to express proliferative cytokines (e.g., IL-12; Pegram et al., 2012) or additional costimulatory ligands (e.g., 4-1BBL; Stephan et al., 2007).

In some embodiments, the presently disclosed subject matter relates to methods of preventing and/or treating a disease, disorder, and/or condition associated with mosaic loss of chromosome Y in blood (mLOY) in a subject in need thereof comprising detecting a missing gene and administering a treatment to a subject with the missing gene. In some embodiments, the missing gene comprises Kdm5d, Uty, Eif2s3y and Ddx3y and any combination thereof.

### Pharmaceutical Compositions and Administration

The presently disclosed subject matter is also directed to methods of administering the compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means. As such, in some embodiments the presently disclosed compositions are administered by injecting the composition subcutaneously, intraperitoneally, into adipose tissue, and/or intramuscularly into the subject.

In accordance with one embodiment, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments, or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion, intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1985; Gennaro, 1990; or Gennaro, 2003; each of which is incorporated herein by reference.

Typically, dosages of the compound of the presently disclosed subject matter which may be administered to an animal, in some embodiments a human, range in amount from 1 g to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

Materials and Methods for the EXAMPLES Mice strains. B6 mouse strain (C57BL/6J) and Rosa26Cas9 knock-in mouse strain (B6(C)-Gt(ROSA)26Sor^{em.1(CAG-cas9*.-EGFP)Rsky}/J) were obtained from The Jackson Laboratory (stock #000664 and #028555, respectively). All mice strains were maintained on a 12-hour light/dark schedule in a specific pathogen-free animal facility and given food and water ad libitum. The protocols for animal experiments described in this paper were approved by the Institutional Animal Care and Use Committee of the University of Virginia.

Plasmids. Single guide RNAs (sgRNA) targeting the centromere of the Y chromosome (gRNA1, gRNA2) were from a previous report (Adikusuma et al., 2017), and the control gRNA is non-targeting. sgRNA sequences are listed in Table 4. Single stranded sgRNA sequences were annealed and cloned into pLKO.sgRNA.EFS.tRFP following restriction digestion with Esp3I, and successful cloning was confirmed by Sanger sequencing with U6 primer (Table 4). Plasmids were from Addgene (pLKO5.0.sgRNA.EFS.tRFP, plasmid #57823; psPAX2; plasmid #12260; and pMD2.G, plasmid #12259).

Ex vivo genome editing of HSPC. sgRNAs were delivered lentivirally to bone marrow lineage-negative cells obtained from ROSA26Cas9 knock-in mice as described previously (Sano et al., 2019).

Lentivirus production. 6-well plates were coated with collagen solution (0.0005%) at 37° C., 5% CO₂ for 30 min. HEK 293T cells were seeded at a density of 1×10⁶ cells per well and incubated at 37° C., 5% CO₂ for 2 hours. The plasmids (0.9 mg of pLKO5.0.sgRNAs.EFS.tRFP, 0.6 mg psPAX2, and 0.3 mg of pMD2.G per well) were co-transfected to HEK293T cells with PEI MAX (Polysciences, 24765-1), and cell culture medium was replaced 3 hours after transfection. Culture supernatant was collected 48 hours after medium change, and centrifuged at 3,000×g for 15 minutes to remove free-floating cells. After filtration through 0.45 mm filter, virus particles were concentrated by ultracentrifugation at a speed of 72,100×g at rₘₐₓ for 3 hours. The virus pellet was suspended with StemSpan medium (Stemcell Technologies, Cat. #09600) without aeration and kept at −80° C. Lentiviral particle titer was determined using a Lenti-X qRT-PCR Titration Kit (Clontech, Cat. #631235).

Isolation of lineage-negative cells and lentivirus transduction. Lineage-negative cells were isolated from the bone marrow of Rosa26Cas9 knock-in mice using a Lineage Cell Depletion Kit (Miltenyi Biotec, Cat. #130-090-858). Cells were pre-incubated with StemSpan medium for 1.5h at 37° C. Lentivirus transduction was performed in the presence of 20 ng/ml of thrombopoietin, 50 ng/ml of stem cell factor 1 and 4 mg/ml of polybrene. Cells were washed and resuspended with RPMI medium before transplantation via the retro-orbital vein.

Bone marrow reconstitution. Due to the nature of this study, male mice were used for BMT recipients in all the experiments.

Whole-body irradiation method. 8- to 12-week-old C57BL/6J recipients were lethally irradiated (5.5Gy twice, 4 hours apart) using an RS-2000 X-ray irradiator (Rad Source Technologies, Inc.). After the second irradiation, each recipient was reconstituted with 0.5 million gene-edited HSPCs as indicated in the manuscript. Sterilized caging, food and water were provided during the first 14 days after transplantation, and water was supplemented with the antibiotics (5 mM sulfamethoxazole, 0.86 mM trimethoprim). To prevent weight loss during the recovery period after irradiation, we used a dietary supplement, DietGel (ClearH2O). Successful engraftment of donor cells was evaluated by flow cytometry one month after bone marrow transplantation.

Fluorescence in situ hybridization (FISH). FISH analysis was performed by collaboration with Cell Line Genetics, Inc. Briefly, slides were incubated in 2×SSC pH 7.2 at 73±1° C. for 2 min. Then, slides were transferred to 0.01 N HCl supplemented with 200 ml of Pepsin Stock Solution and incubated at 37° C. for 13 min. Cells were fixed in 1% formaldehyde for 5 min at room temperature and dehydrated in 70%, 85%, 100% ethanol for 2 min each. Cells were incubated with the commercial probes and slides were placed on the TermoBrite instrument overnight. Slides were immediately transferred to pre-warmed 0.4×SSC/0.3% NP-40 pH 7.2 and washed at 73±0.5° C. for 2 min. The slides were mounted by coverslip using 1:20 Vectashield with DAPI. 200 interphase nuclei were analyzed by two technologists using an Olympus fluorescence microscope (model BX-41).

Karyotype analysis. Lineage-negative cells isolated from mLOY or control mice were immortalized by transducing lentivirus encoding Hoxb8 (LV.T11.Hoxb8.Puro provided from Dr. Reinhold Forster, Hannover Medical School). In this system, Hoxb8 is induced by tetracycline, and the reverse transactivator M2 and puromycin resistance genes are constitutively expressed in bicistronic manner from human phosphoglycerate kinase promoter. Isolated lineage-negative cells were transduced with lentivirus particles (MOI 100) and cultured in StemSpan SFEM medium (STEMCELL technologies, Cat. #09600) supplemented with 50 ng/ml murine SCF (Peprotech, Cat. #250-03), 100 ng/ml human IL-11 (Peprotech, Cat. #200-11), 100 ng/ml human Flt3-Ligand (Peprotech, Cat. #300-19), 20 ng/ml murine IL-3 (Peprotech, Cat. #213-13). Sixteen hours after lentivirus transduction, cells were collected and resuspended in IMDM (Gibco, Cat. #12440053) supplemented with 10% heat inactivated FBS, 1% penicillin-streptomycin, and cytokines (100 ng/ml murine SCF, 100 ng/ml human IL-11, 100 ng/ml human Flt3-Ligand, 20 ng/ml murine IL-3) together with 2.4 g/ml of doxycycline hyclate (Sigma-Aldrich, Cat. #D9891). Immortalized cells were selected by treatment with puromycin (0.5 g/ml) for 48 hours. After transduction and selection by puromycin, RFP-positive fraction was sorted and further cultured for 7 days prior to karyotype analysis. Karyotype analysis was performed by KaryoLogic, Inc. of Durham, NC. Briefly, Colcemid Solution (ThermoFisher, 15212012) was added to the cell cultures at a final concentration of 0.5 g/ml, and incubated at 37° C., 5% CO₂ for 10 minutes. Cells were transferred to centrifuge tubes, spun at 500 g for 7 minutes, then resuspended in 0.075 M KCL hypotonic solution and incubated at room temperature (approximately 22° C.) for 6 minutes. Cells were then spun at 500 g, 7 minutes, and resuspended in 3:1 methanol:acetic acid fixative. Fixed cell suspensions were incubated at room temperature 30 minutes and centrifuged as above. Cell pellets in 0.5 ml of fixative were used to make slides by dropping a single drop onto each wet microscope slide. Slides were baked at 65° C., 20 hours, treated with 0.1% trypsin-EDTA, and stained with Giemsa in Gurr's Buffer at pH 6.8. Metaphase spreads were analyzed at a Leica DM2500 brightfield microscope at 1000×, using Leica Biosystems CytoVision software, version 7.4.

Animal models. mLOY mice. To construct mice deficient in the Y chromosome in blood cells, linage negative cells isolated from ROSA26-Cas9 knock-in mice were transduced with lentivirus vector encoding LOY-gRNA or control-gRNA at the same multiplicity of infection (MOI). 8-12-week-old lethally irradiated male mice were randomly assigned to mLOY group (received LOY-gRNA expressing cells) or control group (received control-gRNA expressing cells). The successful transduction of the gRNAs and engraftment were evaluated by flow cytometric analysis of blood cells at one month after bone marrow transplantation. Control-gRNA was designed to target no specific region of the genome.

Pressure overload model. Transverse aortic constriction (TAC) was performed as previously. Briefly, isoflurane-anesthetized mice were subjected to ligation of the transverse thoracic aorta between the innominate artery and left common carotid artery with a 27-gauge blunt needle; operated mice without constriction served as controls. Surgery for each group of mice was performed by individuals blinded to the identity of the mouse genotype (SS and KH).

Bleomycin-induced lung injury model. Mice received 1 U/kg of bleomycin solution intratracheally (Catalog No. NDC #71288-107-20; Meitheal Pharmaceuticals, Chicago, Illinois, United States of America). Intratracheal instillation was performed in mice that were anesthetized with isoflurane. Mice were intubated with a 20G catheter at an angle of −60° C., and the solution was injected into the trachea by pipet, followed by 150 ml of air.

Myeloid cell depletion. Myeloid cell depletion was achieved by intraperitoneal injection of anti-Gr-1 antibody or control IgG (Catalog Nos. BE0075 and BP0090, respectively; Bio X Cell, Inc., Lebanon, New Hampshire, United States of America). 0.5 mg of antibody per mice was injected every 3 days for 4 weeks. The injections were initiated at three days before TAC operation.

TGFβ signaling inhibition. TGFβ signaling inhibition was achieved by intraperitoneal injection of anti-TGFβ antibody or control IgG (Catalog Nos. BP0057 and BP0083, respectively; Bio X Cell, Inc., Lebanon, New Hampshire, United States of America). 5 mg/kg of antibody per mice was injected every 3 days for 4 weeks. The injections were initiated at three days before TAC operation.

Echocardiography. Cardiac function was assessed using Vevo 2100 ultrasound system equipped with MS550D probe (FUJIFILM VisualSonics, Toronto, Canada). Mice were anesthetized with isoflurane at a concentration of 5% (induction phase) and 1-1.5% (maintenance phase). Each animal was placed on the heating table in a supine position with the extremities tied to the table through four electrocardiography leads. Chest fur was removed with a chemical hair remover, and ultrasound gel was applied to the thorax surface to improve the visibility of the cardiac chambers. Systolic function parameters including posterior wall thickness dimension (PWTd, mm), fractional shortening (FS, %), left ventricular diameter at end-systole (LVDs, mm), and left ventricular diameter at end-diastole (LVDd mm) were measured from M-mode images obtained by short-axis view visualizing both papillary muscles. Apical four-chamber views were obtained for diastolic function measurements using pulse wave Doppler and tissue Doppler imaging at the level of the mitral valve. Parameters collected include peak Doppler blood inflow velocity across the mitral valve during early diastole (E wave), and peak tissue Doppler of myocardial relaxation velocity at the mitral valve annulus during early diastole (e′ wave). Measurements and analysis were performed by two individuals (KH and YW) who were blinded to the identity of the experimental groups of mice using Vevo Lab software (FUJIFILM VisualSonics).

BP measurement. Non-invasive blood pressure measurement of experimental animals was performed using CODA-8 (Kent Scientific Corp., Torrington, Connecticut, United States of America). For acclimation, measurement was performed 3 consecutive days and the result of day 3 was shown. Briefly, mice were placed into animal holders without anesthesia and located on a warming platform to maintain ideal body temperature. Blood pressure was measured by tail cuff and automatically recorded.

Hematological parameters. Peripheral blood cells were obtained from retro-orbital vein and collected into K2EDTA-added BD microtainer blood collection tubes (Catalog No. 365974, BD Biosciences, Franklin Lakes, New Jersey, United States of America). Hematological parameters were analyzed by Element HT5 Veterinary Hematology Analyzer (Heska Corp., Loveland, Colorado, United States of America).

Flow cytometry analysis. The antibodies used for flow cytometric analysis are listed in Table 5. BD LSRFortessa Flow Cytometer (BD Biosciences) was used for data acquisition through the University of Virginia Flow Cytometry Core, RRID: SCR_017829. Data were analyzed with FlowJo Software (BD Biosciences).

Peripheral blood. Peripheral blood cells were obtained from retro-orbital vein and collected into K2EDTA-added BD MICROTAINER® blood collection tubes (BD Biosciences, Cat. #365974). Red blood cells were lysed with EBIOSCIENCE™ 1×RBC Lysis Buffer with occasional vortex (Catalog No. 00-4333-57, Thermo Fisher Scientific, Waltham, Massachusetts, United States of America) for 5 minutes on ice. Incubation with fluorochrome-conjugated antibodies were done for 20 minutes at room temperature in the dark. Cells were defined as; Neutrophil=CD115⁻Ly6G⁺, Ly6C^{hi} Monocyte=CD115⁺Ly6G⁻ Ly6C^{high}, Ly6C^{lo} Monocyte=CD115⁺Ly6G⁻Ly6C^{low}, B cell=CD115⁻Ly6G⁻B220⁺, CD4⁺ T cell=CD115⁻Ly6G⁻CD3e⁺CD4⁺, CD8⁺ T cell=CD115⁻Ly6G⁻CD3e⁺CD8⁺. RFP positive cells were defined using fluorescence minus one control. Heart tissue. Hearts were flushed with 15 ml of cold PBS from apex and excised. Right ventricles were removed, and left ventricles were minced and digested in collagenase I (450 U/ml), collagenase XI (125 U/ml), hyaluronidase (450 U/ml), and DNase I (60 U/ml) (Sigma-Aldrich, Cat. #C0130, C7657, H3506, and D4513, respectively) at 900 rpm at 37° C. for 30 minutes using THERMOMIXER® C (Eppendorf, Framingham, Massachusetts, United States of America). Hearts were subsequently homogenized through a FISHERBRAND™ Cell Strainers. After dead cell staining with Live/Dead Fixable Aqua Dead Cell Stain Kit (Catalog No. L34957, Invitrogen, Waltham, Massachusetts, United States of America), single cell suspension of heart tissue was incubated with fluorochrome-conjugated antibodies for 20 minutes at room temperature. 123count EBEADS™ Counting Beads (Invitrogen, Cat. #01-1234-42) was used for data counting cell numbers. Cells were defined as described below; Neutrophil=CD45⁺CD64⁻Ly6G⁺, Ly6C^{hi} Monocyte=CD45⁺CD64⁺Ly6G⁻Ly6C^{high}, Macrophage=CD45⁺CD64⁺Ly6G⁻Ly6C^{low}, Endothelial cells=CD45⁻CD31⁺mEF-SK4⁻ Fibroblasts=CD45⁻CD31⁻mEF-SK4⁺.RFP positive cells were defined using fluorescence minus one control. Data were analyzed with FlowJo Software.

Histological measurements, Heart tissues were harvested at indicated time points after TAC surgery. Hearts were perfused with cold PBS from apex and fixed in 10% formalin at 4° C. overnight. Samples were processed for paraffin embedding and 7-μm-thick sections were made. Following de-paraffinization and rehydration, hematoxylin-eosin staining was performed using a standard method. Sections were incubated in filtered hematoxylin solution (Catalog No. GHS316, Sigma-Aldrich Corp., St. Louis, Missouri, United States of America) for 2 minutes, washed in tap water, and differentiated in 1% acid alcohol. After dehydration in 70% ethanol, sections were incubated in eosin solution (Sigma, Cat. #HT110116) for few seconds, and dehydrated in a series of ethanol (95% and 100%), cleared in xylene and mount in permanent mounting medium (Vector Laboratories, Cat. #H-5000). Images were taken by a Keyence BZ-X710 microscope to show the global change of heart size. For cardiomyocyte cross-sectional area (CSA) analysis, heart sections were stained with Alexa Fluor 594 conjugated-WGA (Catalog No. W11262, Life Technologies, Carlsbad, California, United States of America). An operator who was blinded to mouse genotype quantified cardiomyocyte CSA by computer-assisted morphometric analysis of microscopy images acquired on a Keyence BZ-X710 microscope. The average CSA of randomly selected 80-100 round-shaped cardiomyocytes per section was used for analysis. For Picrosirius red staining, sections were incubated with freshly prepared staining buffer comprised of 1.2%/w picric acid in water, 0.1%/w Fast Green FCF and 0.1%/w Direct Red 80 solved in PBS (Sigma-Aldrich, Cat. #197378, Cat. #F7252, and Cat. #365548, respectively) for 1 hour at room temperature. Sections were washed briefly in distilled H₂O and dehydrated. The slides were mounted by coverslip using permanent mounting medium. Images were analyzed by Image J software (NIH) for quantification of fibrosis. Myocardial fibrosis size was expressed as a percentage of total LV area.

Lung tissues were harvested, fixed in 10% phosphate buffered formalin, cut, and embedded in paraffin. Sections of 4 m thickness were cut and stained for hematoxylin & eosin (H&E), Masson Trichrome (MT) stain, and Picrosirius red stain (PR). Parenchymal fibrosis was evaluated by Ashcroft score (by 2 separate individuals blinded to the experimental conditions). The area of the red color in the Picro Sirius red stain image in a light microscope was quantified as previously described. The images taken by an BZ-X800 (Keyence) microscope were converted to gray scale, and the total number of white pixels per image was determined as a percentage of the total pixel area using Image J v. 1.8.0, developed for microscopy (USA National Institute of Health, https://imagei.nih.gov/ii/download.html). This procedure was applied to a total of 10 microscopic fields in ×40 magnification per sample, on alveolar septal regions that contained no large airways.

Immunofluorescence staining of heart sections. Following de-paraffinization and rehydration, antigen retrieval was performed in citrate buffer (Vector Labs, #H3300) with microwave oven. Slides were blocked in gelatin from cold water fish skin (Sigma, #G7765) for 1 hour at room temperature. Sections were incubated with primary antibodies either over night at 4° C. (anti-pSMAD2 antibody, Cell Signaling, #3108) or for 30 minutes at room temperature (anti-vimentin antibody, R&D Systems, #MAB2105). After washing, sections were incubated with fluorescence-conjugated secondary antibodies for 1 hour at room temperature. DAPI was used for nuclei staining. A total of 10 fields per section were taken using Leica SP8 Confocal Microscope and the number of positive cells (stained with anti-pSMAD2 antibody or anti-vimentin antibody) were manually counted. The number and percentage of positive cells in sections were calculated by the average of 10 fields. Fibroblasts were defined as vimentin positive cells. Staining was performed with the support of the core histology facility at the University of Virginia, Charlottesville, Virginia, United States of America.

Biochemical analyses. Mouse serum was collected from clotted blood. Briefly, peripheral blood was obtained from the retro-orbital vein and collected into clot activator- and serum separation gel-added BD SST™ MICROTAINER© tubes (BD Biosciences, Cat. #365967). Tubes were inverted five times, allowed 30 minutes clotting time, and centrifuged for 10 minutes at 6000 rcf (g), leaving the serum above the gel. Serum was snap frozen and kept at −80° C. until use. Serum brain natriuretic peptide (BNP) was quantified by the BNP Enzyme Immunoassay Kit according to the manufacturer's instructions (Catalog No. EIAM-BNP-1, RayBiotech Inc., Peachtree Corners, Georgia, United States of America). Serum levels of Renin 1 and Angiotensin II were measured using the mouse Renin 1 ELISA kit (RayBiotech, Cat. #ELM-Renin1-1) and mouse Angiotensin II EIA kit (Sigma-Aldrich, Cat. #RAB0010-1KT) according to the manufacturer's protocols.

### Transcript Expression Analysis,

Bulk RNA sequencing. For bulk RNA-Seq analysis of the peripheral blood neutrophils, whole blood was collected and incubated with RBC lysis buffer as described above in the section “Flow cytometry analysis.” After staining with fluorescent-labeled monoclonal antibodies, a total of 10,000 blood neutrophils (Ly6G⁺CD115⁻RFP⁺) were sorted directly into 350 ul of buffer RLT (Qiagen, Cat. #79216) containing 1% b-mercaptoethanol using Influx Cell Sorter platform with 100 mm nozzle and flow pressure set to 20 psi (BD Biosciences). For cardiac macrophages and neutrophils, heart digests were prepared as described above. A total of 6,000 macrophages (CD45.2⁺Ly6G⁻CD64⁺RFP⁺) or 800 cardiac neutrophils (CD45.2⁺Ly6G⁺CD64-RFP⁺) were sorted in the same setting as blood neutrophils. RNA extraction, RNA library preparations, sequencing reactions, and initial bioinformatics analysis were conducted at Genewiz, LLC. Total RNA was extracted using RNEASY® Plus Mini Kit (Qiagen). RNA was quantified using Qubit Fluorometer (Life Technologies) and RNA integrity was checked with TapeStation (Agilent Technologies). SMART-Seq v4 Ultra Low Input Kit for Sequencing was used for full-length cDNA synthesis and amplification (Clontech), and Illumina Nextera XT library was used for sequencing library preparation. Briefly, cDNA was fragmented, and adaptor was added using Transposase, followed by limited-cycle PCR to enrich and add index to the cDNA fragments. The final library was assessed with Agilent TapeStation. The sequencing libraries were multiplexed and clustered on one lane of a flowcell. After clustering, the flowcell was loaded on the Illumina HiSeq instrument. The samples were sequenced using a 2×150 Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mis-match was allowed for index sequence identification. The analysis of RNAseq data was performed with two pipelines. In the initial analysis, sequence reads were trimmed to remove possible adapter sequences and nucleotides with poor quality using Trimmomatic v.0.36. The trimmed reads were mapped to the Mus musculus GRCm38 reference genome available on ENSEMBL using the STAR aligner v.2.5.2b. Unique gene hit counts were calculated by using featureCounts from the Subread package v.1.5.2. After extraction of gene hit counts, the gene hit counts table was used for downstream differential expression analysis. A comparison of gene expression between groups of samples was performed using DESeq2. The Wald test was used to generate p-values and log 2 fold changes. Genes with an adjusted p-value <0.05 and absolute log 2 fold change >1 were called as differentially expressed genes for each comparison. In the alternative analysis, sequence trimming was performed with Trim Galore! (Babraham Bioinformatics, Babraham Institute, Cambridge, England) and sequence alignment and quantitation were performed by Kallisto 0.44.0. A Mus musculus GRCm38 FASTA transcriptome file from Ensembl database was used as an index for alignment. Differential gene expression analysis was performed using DEseq2 as described above. For the identification of statistically enriched pathways among the differentially expressed genes identified by RNA-Seq analysis, gene set enrichment analysis (GSEA) was performed. Data were deposited into the National Center for Biotechnology Information's Gene Expression Omnibus database under accession number GSExxx.

Single cell RNA sequencing. Cardiac immune cells derived from BMT were isolated from control and mLOY mice at 7 days post-TAC. Hearts were isolated from mice, tissues were minced and digested as described in the Flow cytometry method above, and viable BMT-derived cardiac immune (DAPI⁻CD45⁺RFP⁺) cells were FACS-isolated (n=3 per condition, pooled samples after FACS). Single cell suspensions of ˜8,000 cells per sample were then used to generate single cell RNA sequencing libraries (Chromium Single Cell v3.1 3′ Reagent Kit, 1Ox Genomics), and nucleotide reads were generated by paired-end next-generation sequencing (P2 2×100 Sequencing Kit, NextSeq 2000 Sequencer, Illumina). Library preparation and sequencing was performed according to validated standard operating procedures established by the University of Virginia Genome Analysis and Technology Core, RRID: SCR_018883. Sequencing of the single cell datasets yielded 7,276 control cells with 45,202 reads per cell and 1,800 genes per cell, and 5,475 knockout cells with 23,360 reads per cell and 1,247 genes per cell. Poor quality cells were filtered out with slightly different cutoffs in each sample related to the depth of sequencing coverage: control cells with fewer than 200 genes and greater than 4000 genes, fewer than 20000 reads, or percentage of mitochondrial reads greater than 10% were filtered out; mLOY cells with fewer than 200 genes and greater than 3000 genes, fewer than 8000 reads, or percentage of mitochondrial reads greater than 10% were filtered out. The SCTransform algorithm was used to normalize and scale the control and mLOY datasets before integration following the standard Seurat v4.0.4 vignette. Control and mLOY datasets were normalized and scaled, then combined through SCTransform integration through the standard Seurat v4.0.4 computational pipeline. UMAP dimensionality reduction was performed using 30 principal components, and cells were clustered using resolution=0.5. Gene expression was then re-normalized and re-scaled within the integrated dataset. Clusters were annotated based on expression of known markers: B cells (Cd79a), T cells (Cd3d), NK cells (Klrk1), Neutrophils (Csf3r), Dendritic cells (Cd209a), Monocytes/Macrophages (Cd68). Within the Monocytes/Macrophages population, sub-populations were identified by known markers: Proliferative (Mki67), Monocytes (Ly6c2), Pro-Inflammatory (Il1b, Ccr2^{hi}), and Pro-Fibrotic (Mrc1, Lyve1), with Non-Activated lacking enrichment for specific markers. Differentiation trajectory analysis was performed using the PHATE v1.0.7 R package, with parameters knn=20 and t=20. Gene regulatory network analysis was performed using the SCENIC v1.2.4 computational pipeline. Data were deposited into the National Center for Biotechnology Information's Gene Expression Omnibus database under accession number GSExxx.

Real-time PCR. Heart tissues were homogenized and lysed in a QIAZOL® Lysis reagent (Qiagen, Cat. #79306), then total RNA was purified using QIAcube with RNEASY® Mini QIAcube Kit (Qiagen, Cat. #74116). Total RNA from cells was isolated using RNEASY® Mini Kit or RNEASY® Micro Kit (Qiagen, Cat. #74104 and Cat. #74004, respectively). 1 μg of isolated RNA was used for reverse transcription with High-Capacity cDNA Reverse Transcription Kit (Thermo Fisher Scientific, Cat. #4368814). Quantitative real-time PCR (qRT-PCR) was performed with Power SYBRac Green PCR Master Mix (Applied Biosystems, Cat. #4367659) using a QuantStudio 6 Flex PCR system (Applied Biosystems). Primers for mouse gene expression studies are shown in the Table 6. 36b4 was used as a reference gene for normalization. Gene expression was evaluated with DDCT method.

Immunoblot analysis. Heart tissues were homogenized and lysed in lysis buffer containing 10 mM Tris, 150 mM NaCl, 5 mM EDTA, 1% Triton-X, proteinase inhibitor cocktail (Thermo Scientific, Cat. #87786) and phosphatase inhibitor cocktail (Thermo Scientific, Cat. #78420). Equal amounts of protein were used for SDS-PAGE and transferred to PVDF membranes. Membranes were incubated with the indicated primary antibodies (TGFβ1, Abcam, ab215715; pSMAD2, Cell Signaling, #3108; SMAD2/3, Cell Signaling, #8685 and β-actin, Cell Signaling, #4970S), followed by incubation with secondary antibodies conjugated with horseradish peroxidase. Signals were detected using ECL Prime System (GE Healthcare, Cat. #RPN2232).

### Example 1

### Age-Related Pathologies are Accelerated in Mice with mLOY in Blood

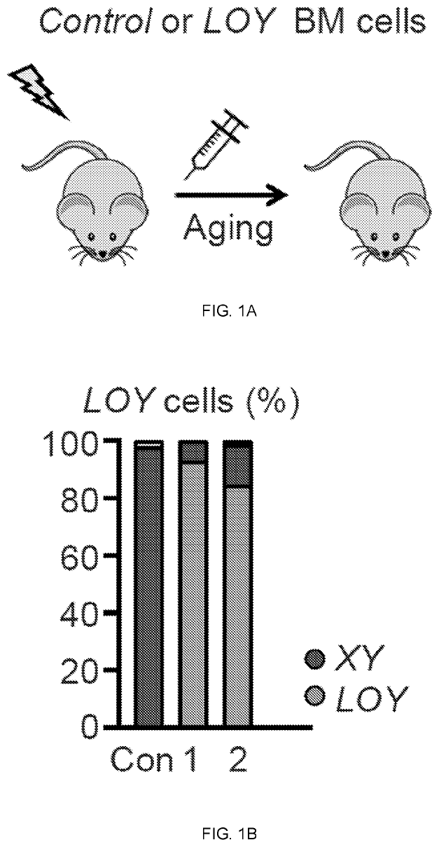

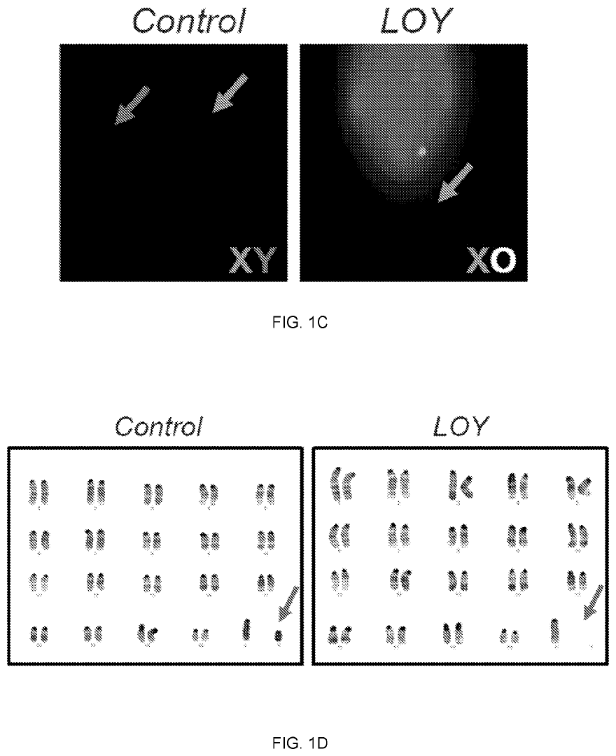

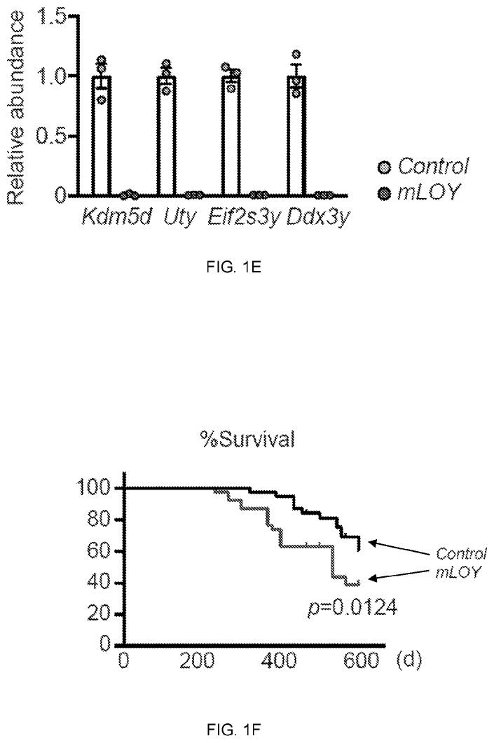

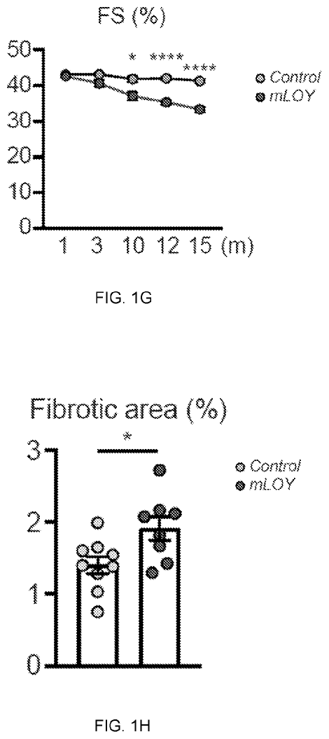

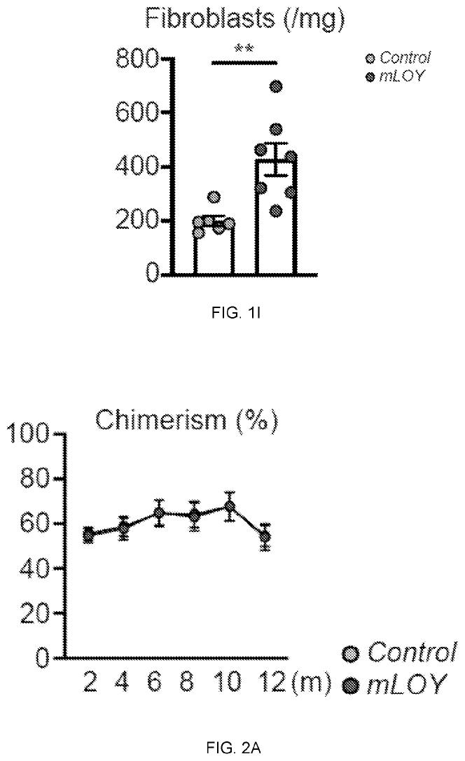

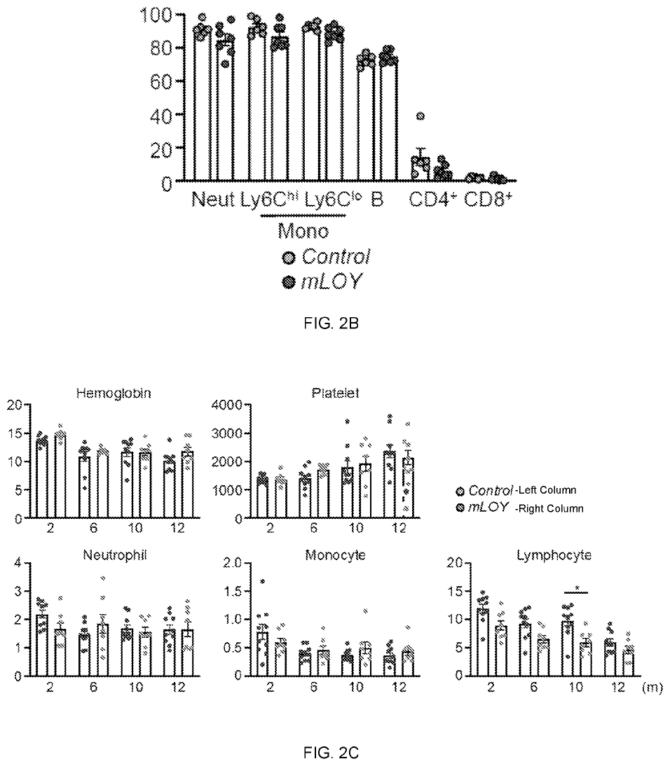

We constructed a mouse model of hematopoietic mLOY by employing CRISPR/Cas9 gene editing to target repeat DNA sequences that are specific to the centromere of the Y chromosome. Guide RNAs (gRNA) and a tRFP marker were delivered to lineage-negative bone marrow cells via lentivirus vector prior to transplantation to lethally-irradiated wild-type mice (FIG. 1A). To decouple the Cas9 gene from lentivirus vector and maximize gene editing efficiency, donor bone marrow cells were isolated from ROSA26-Cas9 knock-in mice that express the Cas9 endonuclease in a ubiquitous manner (Sano et al., 2019). Two LOY-gRNAs (LOY-gRNA1 and 2) targeting different repeat sequences within the centromere were evaluated for the efficiency of Y chromosome ablation by fluorescent in situ hybridization (FISH) analysis of X and Y chromosome in tRFP-positive blood cells collected from mice reconstituted with bone marrow cells. As a control, lentivirus encoding a gRNA designed to not target any region of the genome was employed in the ROSA26-Cas9 knock-in donor cells. Y chromosome ablation efficiency was approximately 95% and 80% for LOY-gRNA1 and LOY-gRNA2, respectively (FIGS. 1B and 1C). Thus, unless otherwise indicated, the mouse model of mLOY employed a lentivirus vector that expresses the LOY-gRNA1 transcript and the tRFP⁺ marker protein to assess cell transduction. Y chromosome ablation in vivo was validated by karyotype analysis using lineage-negative bone marrow cells isolated from mLOY and control mice that were immortalized by lentivirus-mediated HoxB8 overexpression (FIG. 1D). Consistent with the ablation of the male sex chromosome, the Y chromosome-encoded transcripts Kdm5d, Uty, Eif2s3y and Ddx3y were not detectable in the circulating tRFP⁺ leukocytes of the mLOY mice (FIG. 1E). Due to the inefficiency of bone marrow progenitor cell transduction by the lentivirus vector, blood chimerism defined as percentage of tRFP-positive white blood cells ranged from 49 to 81% in various experiments (mean=64.9±4.0%), in line with levels of mLOY in males that have been associated with various disease processes (Forsberg et al., 2014; Dumanski et al., 2016). These levels of chimerism were maintained for 12 months (FIG. 2A). Focusing on each immune cell population, chimerism was higher in myeloid cells compared to B and T cells, consistent with observations of mLOY in males (Dumanski et al., 2021; FIG. 2B).

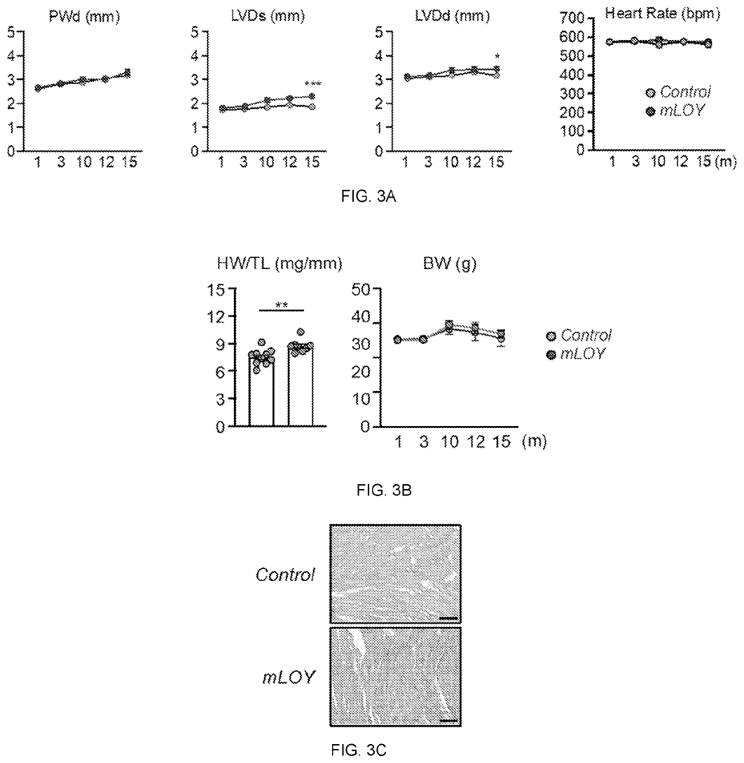

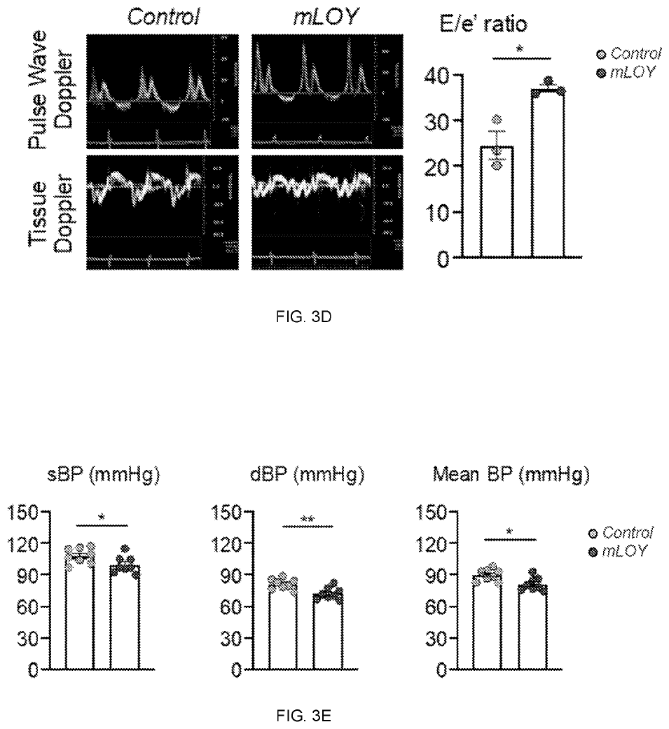

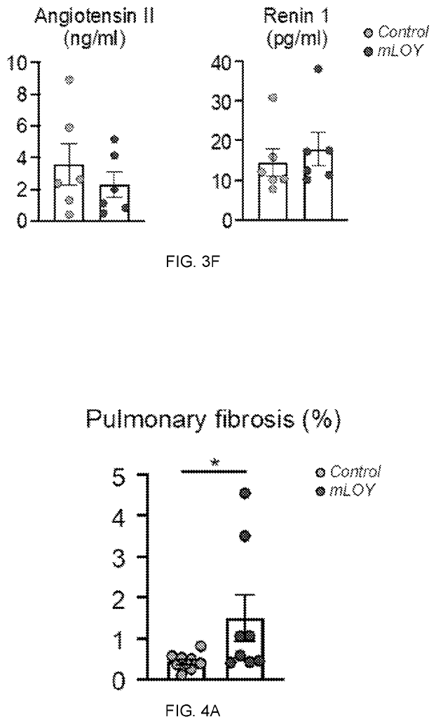

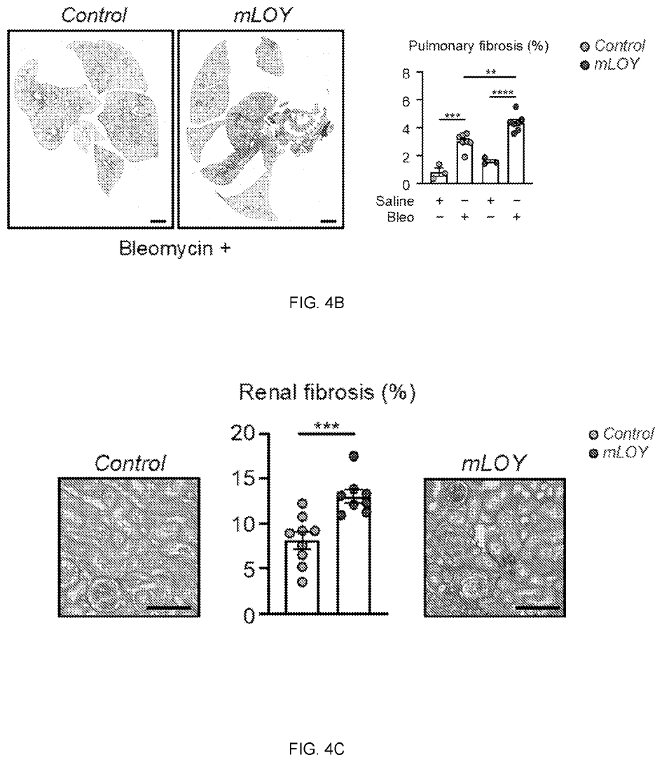

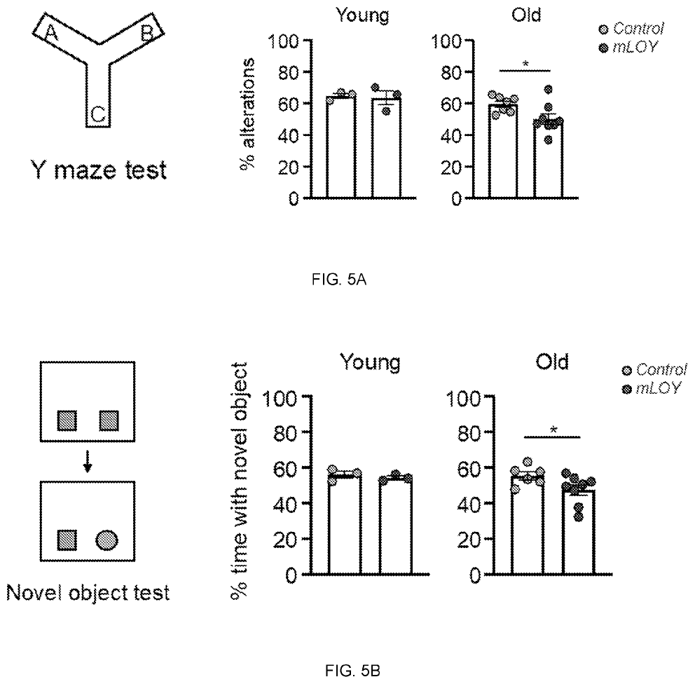

The phenotypic consequences of mLOY were evaluated in aging mice. No obvious hematological abnormalities were observed in mLOY and control mice during the follow-up period (FIG. 2C). However, mice with the mLOY condition displayed significantly shorter lifespans compared with control mice (FIG. 1F). Serial echocardiographic analyses revealed the development of an accelerated age-associated cardiomyopathy in the LOY mice, with greater cardiac dysfunction detected in the older mice (FIGS. 1G and 3A). Consistent with the development of an age-associated cardiomyopathy, mLOY mice displayed a small increase in heart mass at the termination of the experiment, but there were no differences in body weights between mLOY and control genotypes over the course of the experiment (FIG. 3B). Notably, the mLOY condition was associated with an increase in myocardial fibrotic area (FIG. 1H), determined by the quantitative analysis with Picrosirius red staining of cardiac sections (FIG. 3C), and an increase in the quantity of myocardial MEF-SK4⁺ fibroblasts as assessed by flow cytometry (FIG. 1I). Consistent with this elevated fibrotic response, the mLOY condition led to elevated left ventricular filling pressure (E/e′) that is indicative of diastolic dysfunction (FIG. 3D). These cardiac changes were observed despite modest reductions in blood pressure in the mLOY mice (FIG. 3E), and no changes in the serum levels of Renin 1 and Angiotensin II (FIG. 3F). At the 15-month timepoint post-BMT, mLOY mice also showed accelerated fibrotic response in the lung interstitium (FIG. 4A), and greater pulmonary fibrosis could also be observed in young mice after the intratracheal administration of bleomycin (FIG. 4B). Histological analysis of kidney also revealed a greater degree of fibrosis in the mLOY mice compared to control mice at the 15-months post-BMT (FIG. 4C). Finally, assessments of cognitive function revealed that aging mLOY mice (15 months post-BMT) have short-term working memory deficits in the Y-maze and novel object recognition test, but these phenotypes were not observed in young mLOY mice (2 months post-BMT; FIGS. 5A and 5B). Collectively, these results indicate that mLOY mice can recapitulate aspects of the mLOY phenotype observed in males and suggest that accelerated tissue fibrosis could be a mechanistic feature of this condition. Because a potential relationship between mLOY and cardiac dysfunction has not been reported previously, further investigations focused on the role of mLOY in heart failure.

### Example 2

### Mloy Worsens the Outcome of Experimental Heart Failure in Mice

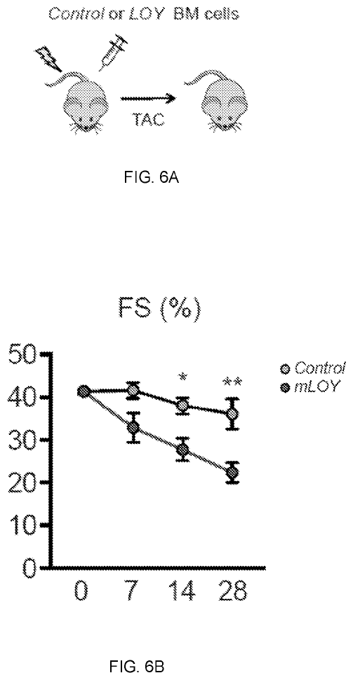

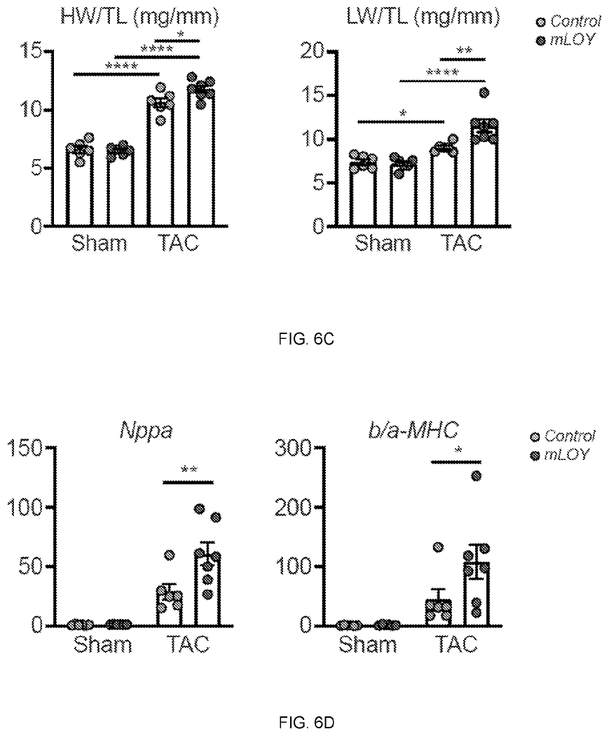

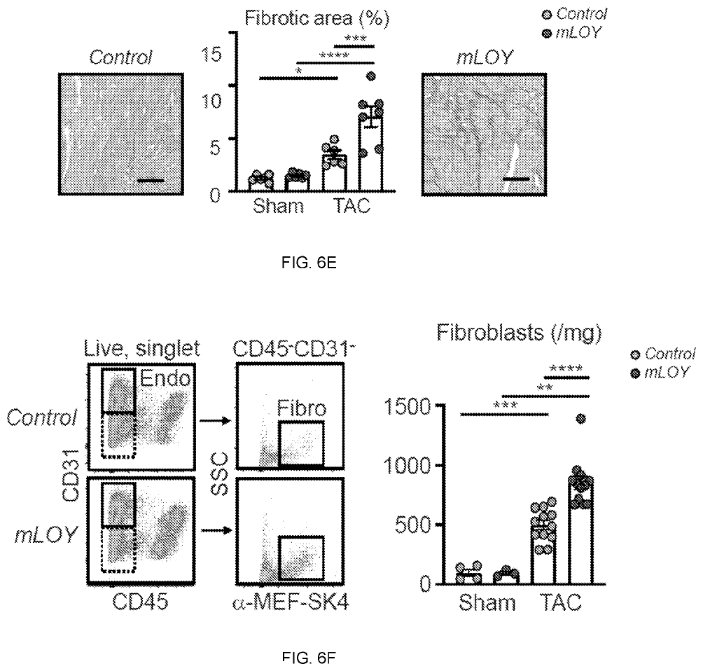

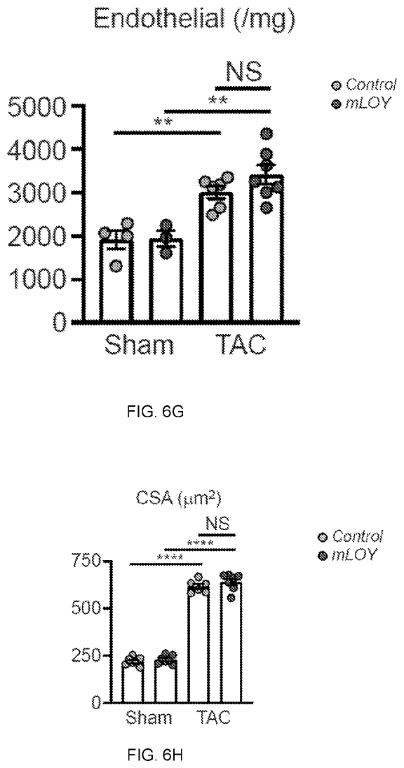

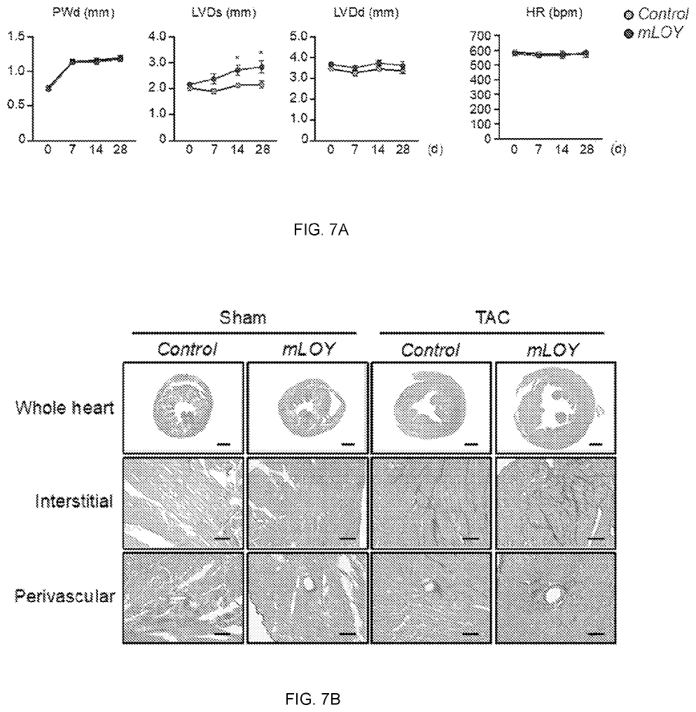

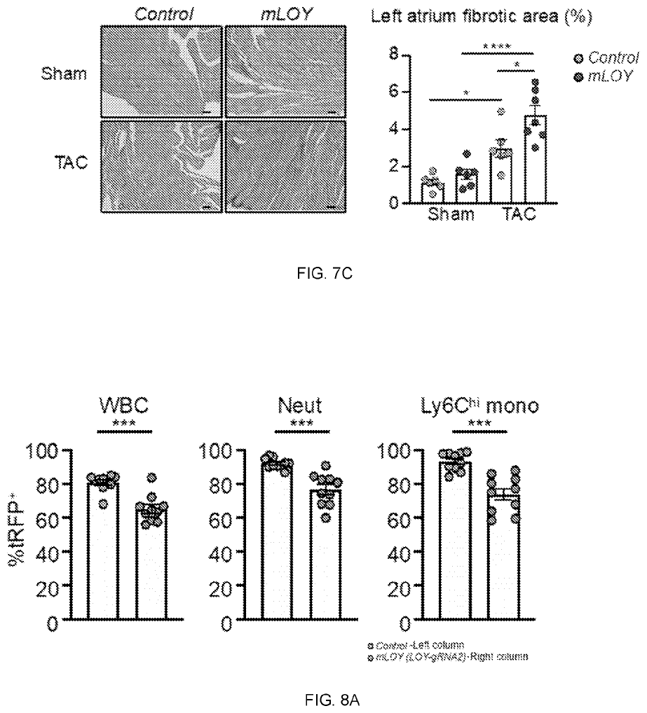

To further investigate the relationship between mLOY and cardiac disfunction, 12- to 16-week-old (4 weeks after BMT) mice were subjected to transverse aortic constriction (TAC) surgery (FIG. 6A). TAC represents a model of pressure overload cardiac dysfunction that is prevalent in the elderly and involves prominent myocardial fibrosis. In agreement with observations in the older unchallenged mice, echocardiographic analysis revealed a greater progressive decline in cardiac function in mLOY mice compared to control mice following TAC (FIGS. 6B and 7A). Consistent with these data, mLOY mice displayed greater increases in heart weight-to-tibia length as well as lung weight-to-tibia length ratios (FIG. 6C) that is indicative of lung congestion that can result from cardiac dysfunction. Transcripts encoding atrial natriuretic peptide A and the ratio of transcripts encoding myosin heavy chain R/a, markers of advanced heart failure, were significantly upregulated in the hearts from mLOY compared to control mice (FIG. 6D). Histological analysis revealed greater interstitial and perivascular fibrosis in both the left ventricle and atrium of LOY mice after TAC (FIG. 6E). Consistent with the increase in fibrosis, flow cytometric analysis of heart cells revealed that the number of MEF-SK4⁺ fibroblasts was significantly higher in the hearts from mLOY mice compared to control (FIG. 6F). In contrast, there was no detectable difference in the number of cardiac endothelial cells or the average myocyte cross-sectional area between the experimental groups (FIGS. 6G and 6H), indicating that the predominant cellular effect of mLOY is on the fibroblast content of the heart.

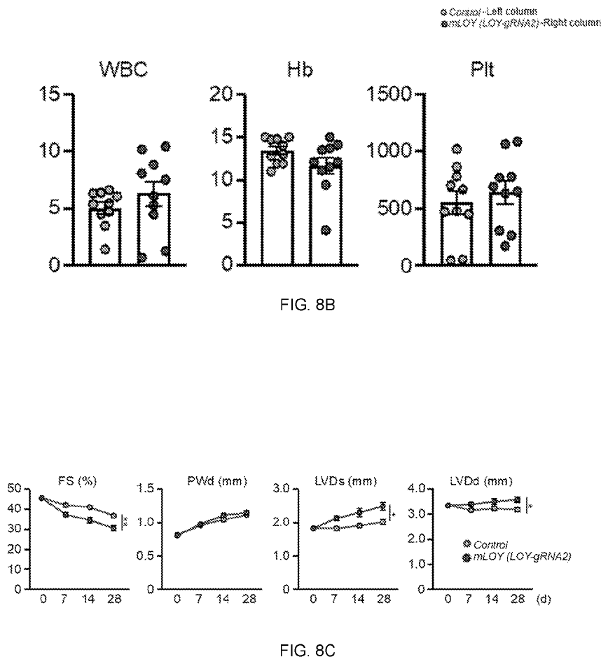

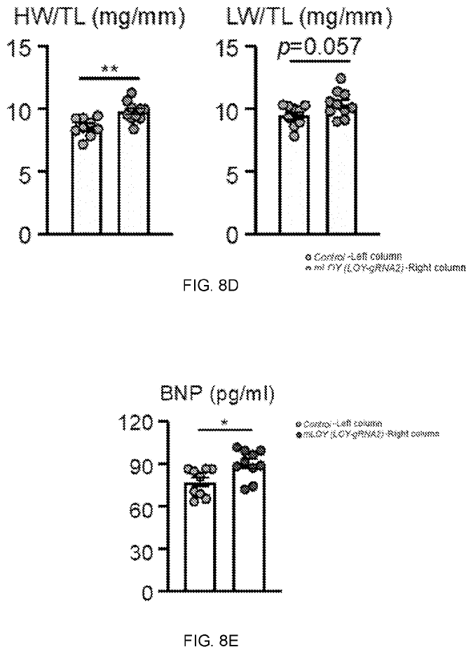

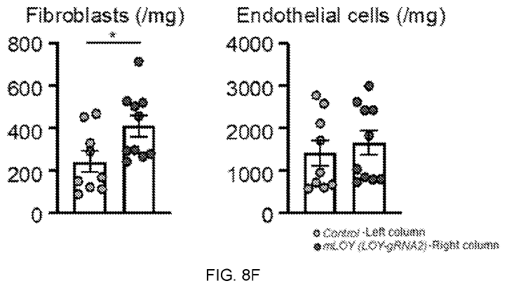

Accelerated cardiac disfunction could also be demonstrated in mice transplanted with lineage-negative bone marrow cells that were transduced with a gRNA that targets a different centromeric repeat in the Y chromosome (mLOY-gRNA2). The efficiency of Y chromosome ablation achieved by mLOY-gRNA2 was comparable but slightly less than that of mLOY-gRNA1 (FIGS. 1B and 8A). Transduction with mLOY-gRNA2 did not affect white blood cell counts, hemoglobin, or platelet levels (FIG. 8B). However, TAC surgery led to greater cardiac dysfunction, increased heart weights, higher concentrations of serum BNP, and higher numbers of cardiac fibroblasts were observed in the mLOY-gRNA2-treated compared to control mice (FIGS. 8C-8F). These data corroborate the results with the mLOY-gRNA1 reagent and provide additional support for the finding that Y chromosome deletion in blood cells contributes to cardiac dysfunction and an accelerated fibrotic response.

### Example 3

### Y Chromosome Deficiency Modulates the Transcriptional Profile of Cardiac Macrophages

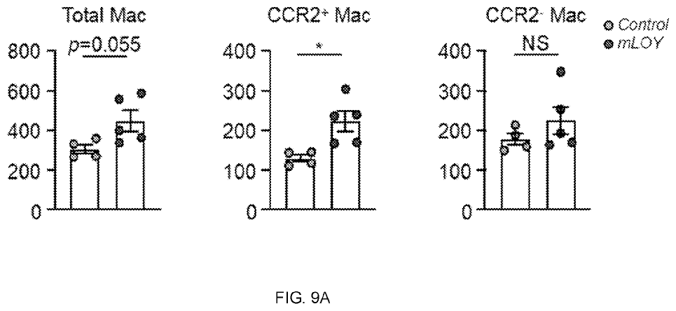

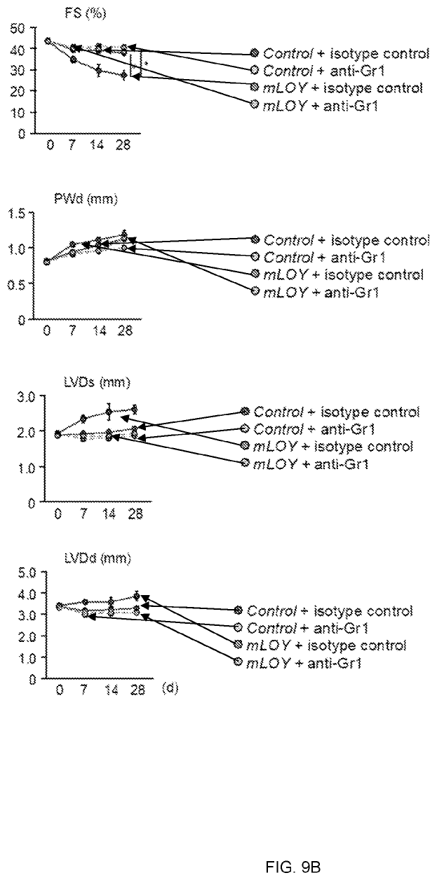

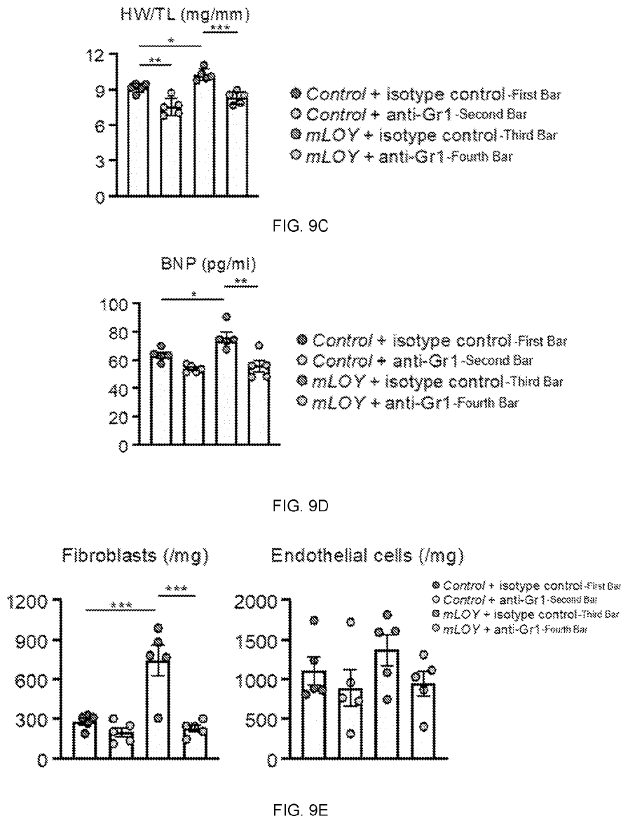

Myeloid cells typically display the greatest extents of Y chromosome deficiency in the blood of males (Dumanski et al., 2021). In the experimental model, analysis of the cardiac immune cell populations revealed significantly higher numbers of CCR2⁺ cardiac macrophages in TAC-treated hearts from mLOY-gRNA1 mice compared to those from control mice (FIG. 9A), suggesting that cardiac macrophages derived from Y chromosome-deficient hematopoietic stem cells have altered functional properties. Thus, to test whether Y chromosome deficiency in myeloid cells account for the accelerated heart failure phenotype, anti-Gr1 antibody, which blocks neutrophil and monocyte recruitment in injured tissue, was administered to the different experimental groups of mice (Daley et al., 2008). In mLOY mice that underwent TAC surgery, treatment with anti-Gr1 antibody attenuated the accelerated cardiac dysfunction (FIG. 9B) and reversed the elevations in heart weight and serum BNP (FIGS. 9C and 9D). Treatment with anti-Gr1 antibody also reversed the increase of the number of cardiac fibroblasts observed in the mLOY mice, but did not affect the quantity of vascular endothelial cells (FIG. 9E). Collectively, these results suggest that loss of Y chromosome in myeloid cells can largely account for the pathological cardiac phenotype in mLOY mouse model.

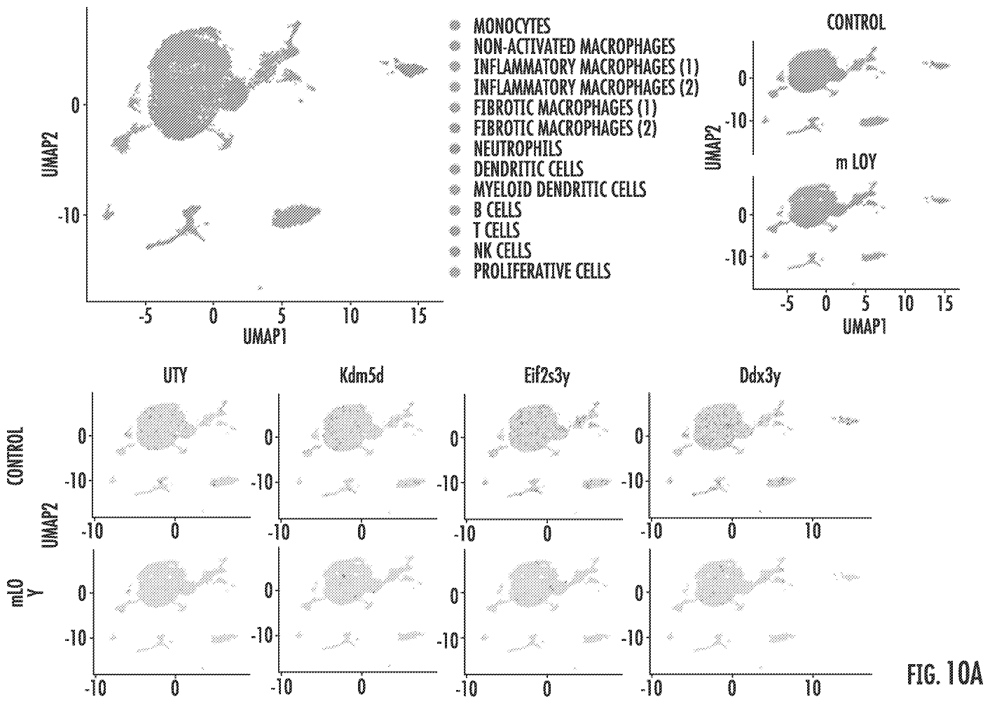

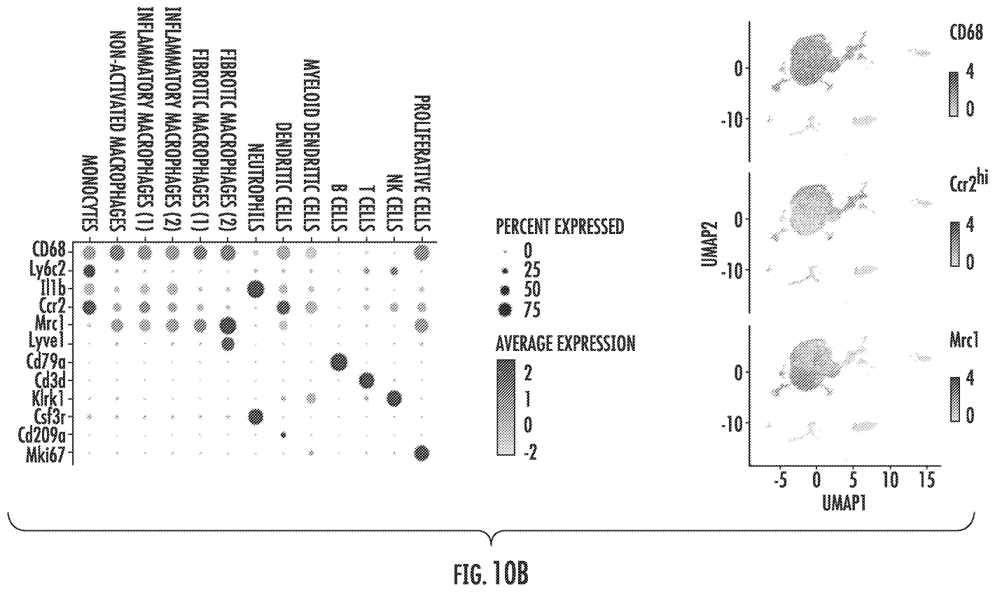

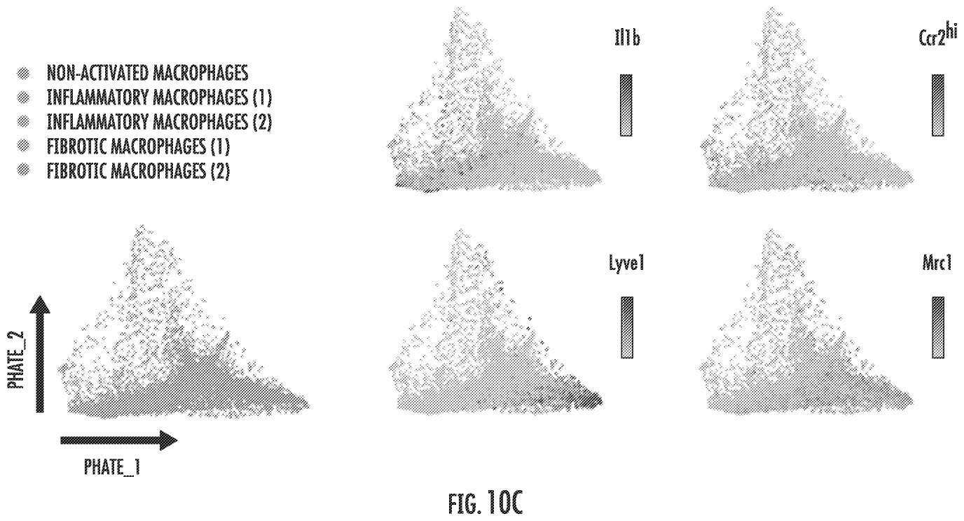

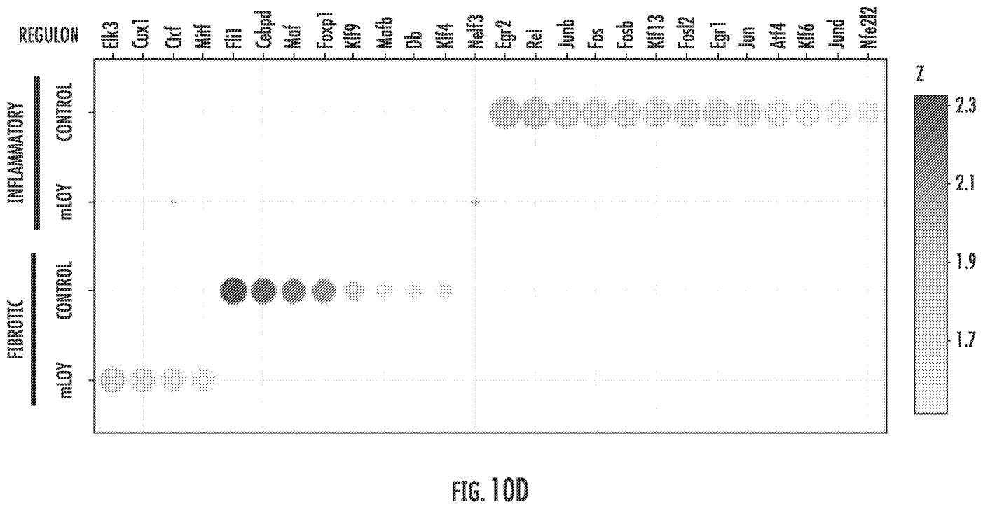

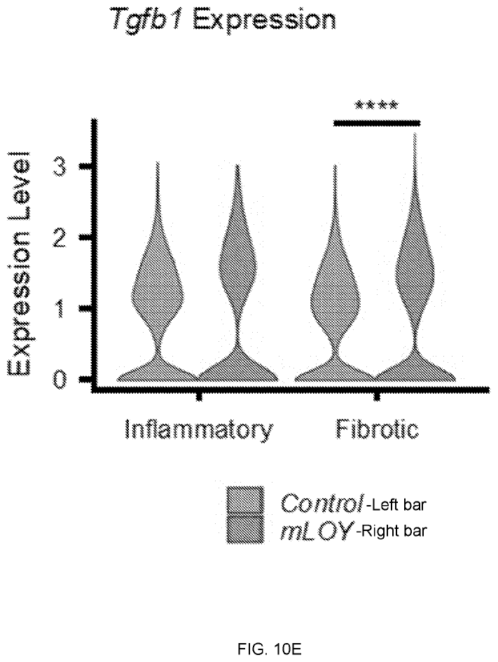

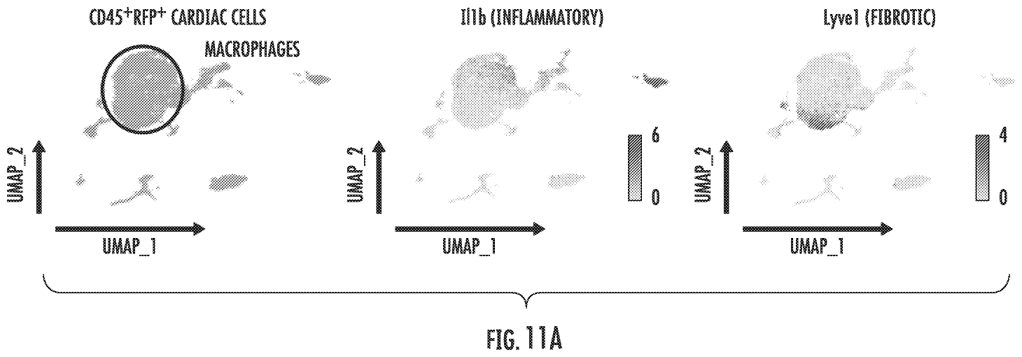

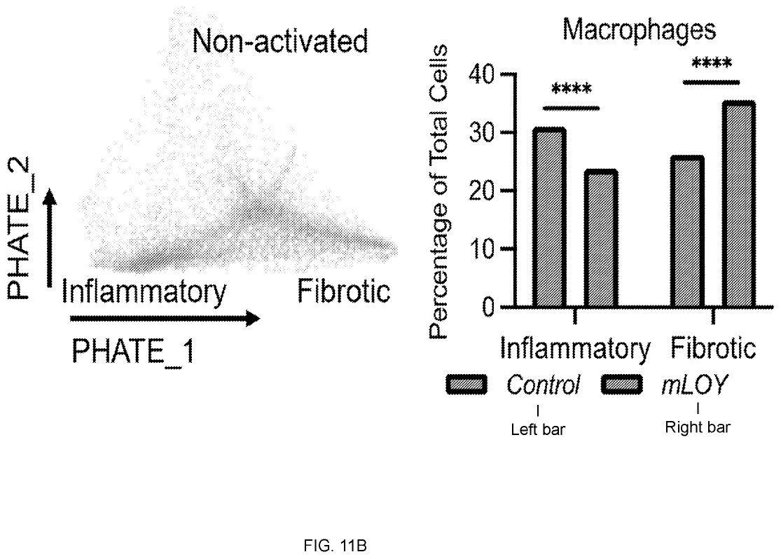

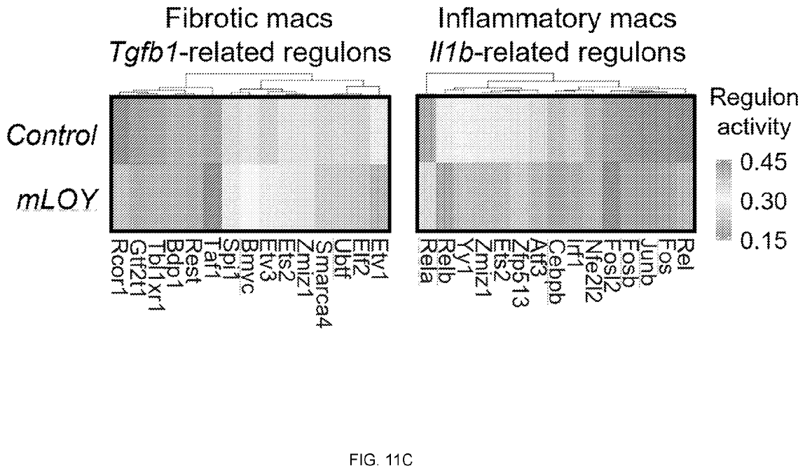

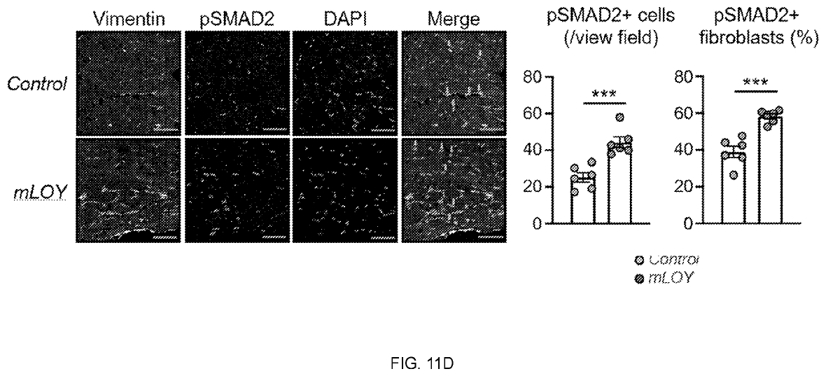

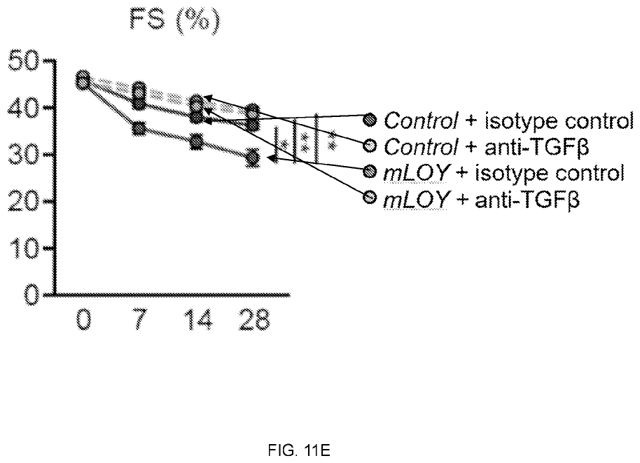

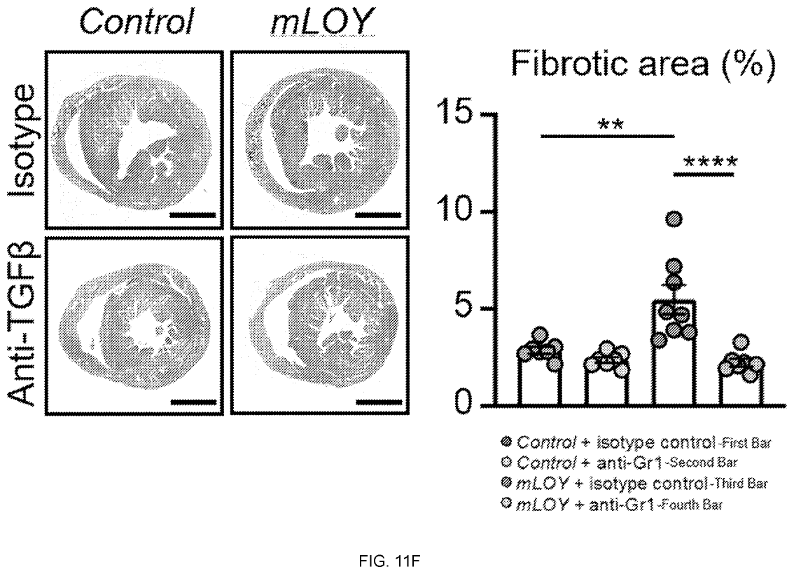

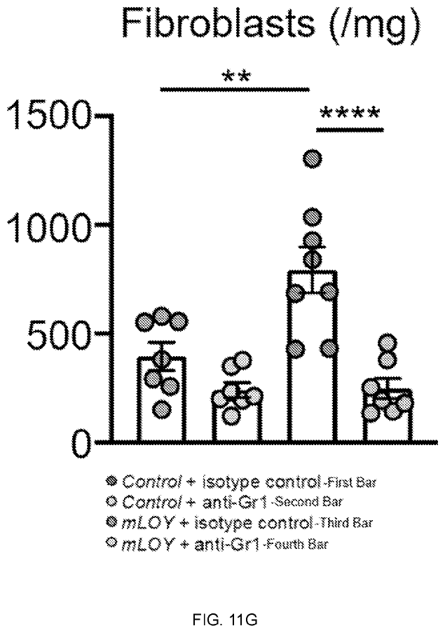

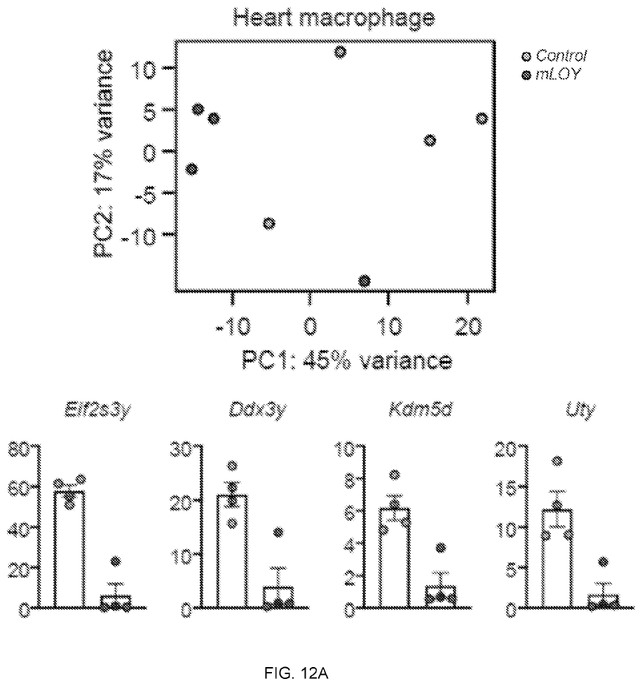

To address mechanistic aspects of mLOY-mediated cardiac dysfunction, single cell RNA sequencing (scRNAseq) was performed on BMT-derived (RFP⁺) immune cells (CD45+) at 7 days post-TAC from mLOY and control mice. Seurat analysis of gene expression enabled the clustering of immune cell populations, and the reduced expression of Y chromosome-encoded genes could be detected in various clusters (FIG. 10A). CD68⁺ macrophages accounted for the largest portion of immune cells and they displayed polarization based upon the expression of the marker genes Il1b and Ccr2^{hi}, that will henceforth define an inflammatory subpopulation, and the marker genes Lyve1 and Mrc1 that will henceforth define a fibrotic subpopulation (FIG. 11A and FIG. 10B). PHATE analysis, to assess differentiation progression and branching, revealed a trajectory from non-activated macrophages to a continuum of inflammatory and fibrotic macrophages (FIG. 10C), with the fibrotic macrophage subpopulation containing a greater percentage of mLOY cells and the inflammatory macrophage subpopulation containing a greater percentage of control cells (FIG. 11B). Consistent with the interpretation that mLOY promotes macrophage polarization toward a fibrotic phenotype, analysis with the SCENIC regulatory gene network algorithm revealed that profibrotic regulons were enriched by the mLOY condition in the fibrotic macrophages (FIG. 10D). Conversely, pro-inflammatory regulons were suppressed by the mLOY condition in the inflammatory macrophage subpopulation. Further analyses revealed that the mLOY condition promoted the enrichment of regulons specifically associated with TGFB signaling in the fibrotic macrophage subpopulation, while regulons associated with IL1β signaling were downregulated by mLOY in the inflammatory subpopulation (FIG. 11C). Analysis of the TGFB1 transcript revealed that the mLOY condition promoted the expression of this transcript in the fibrotic macrophages, but not in the inflammatory macrophages (FIG. 10E).

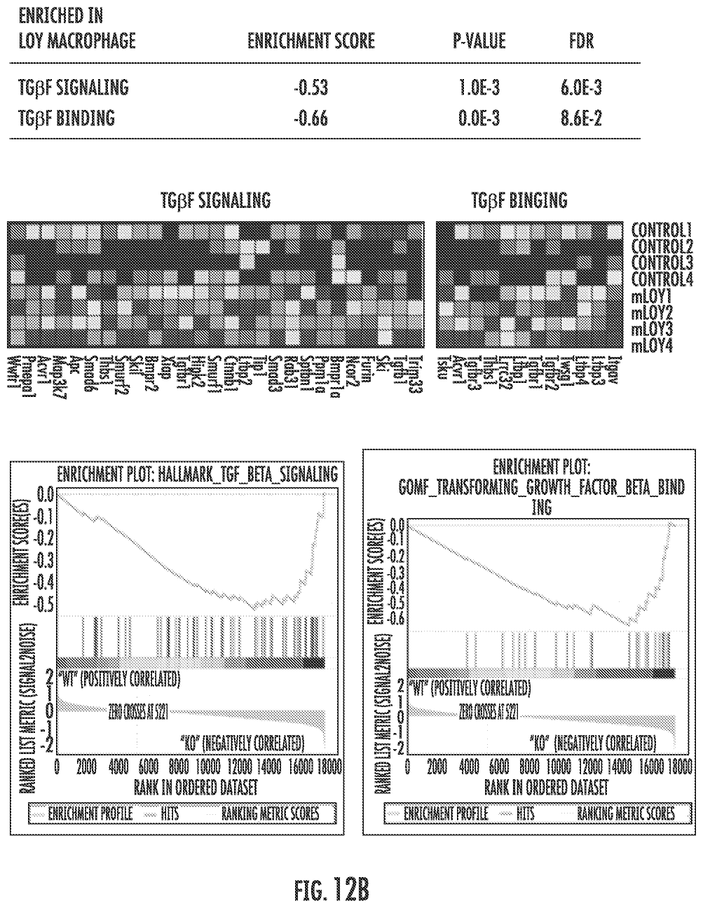

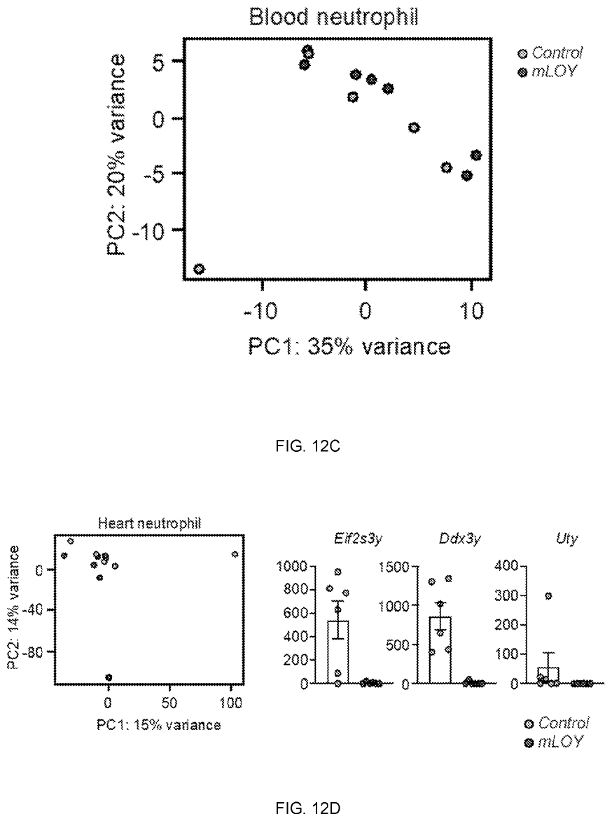

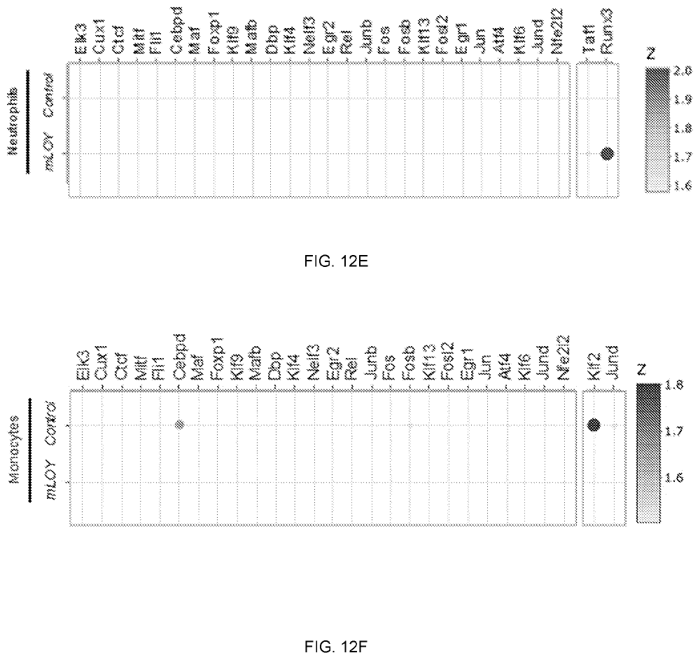

The phenotypic transition of Y chromosome-deficient cardiac macrophages was also evident in the bulk transcriptome analysis of BMT-derived (RFP⁺) cardiac macrophages. As shown in the principal components analysis (PCA) plots, mLOY macrophages isolated from heart displayed a distinct transcriptomic profile from control cardiac macrophages (FIG. 12A). The gene set enrichment analysis (GSEA) revealed that differentially expressed transcripts related to “TGFβ signaling,” were enriched in mLOY macrophages (FIG. 12B). In addition, GSEA revealed differentially expressed transcripts related to “TGFβ binding,” including LTBP1, LTBP3 and LTBP4, that facilitate the localization, secretion, and activation of TGFβ, were upregulated in macrophages in the mLOY condition. In contrast, bulk or single cell analysis revealed little or no differences in transcriptomes of cardiac monocytes, cardiac neutrophils or blood neutrophils between LOY and control conditions (FIGS. 12C-12F).

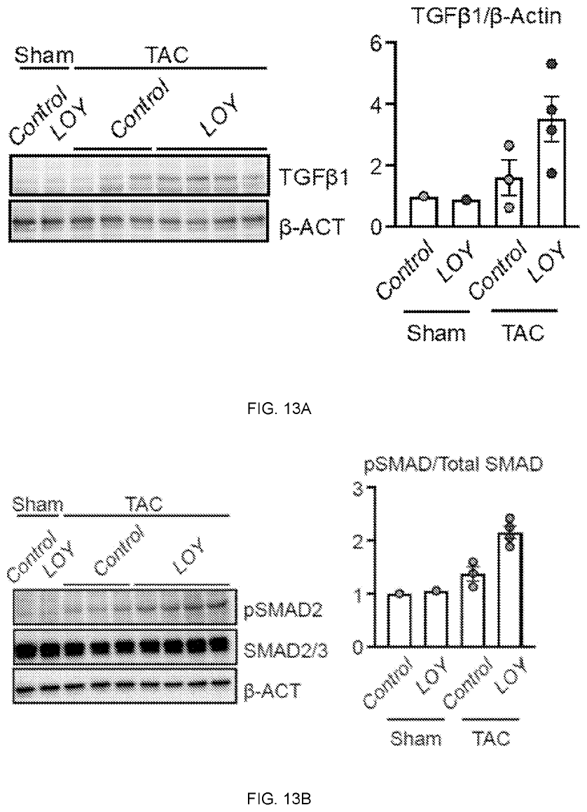

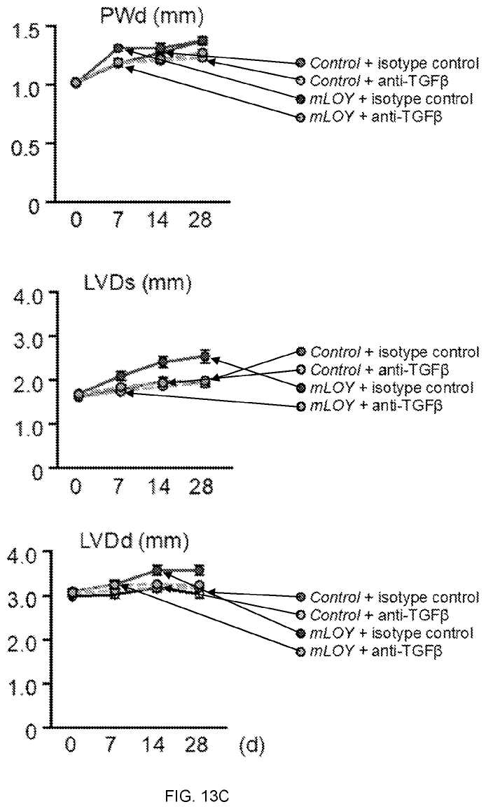

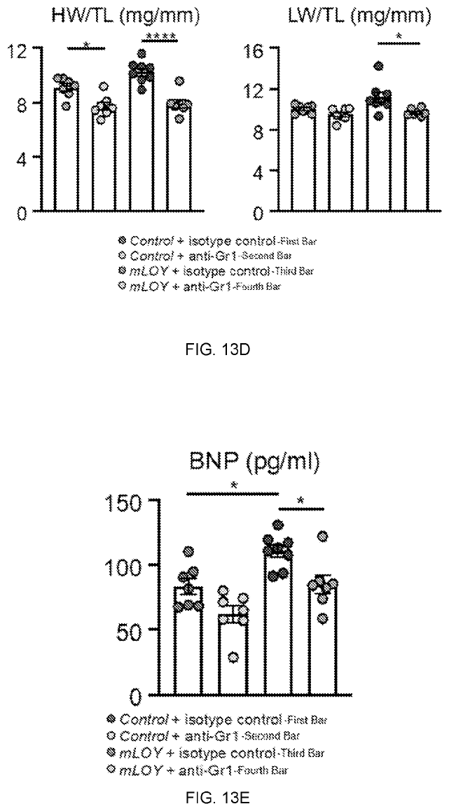

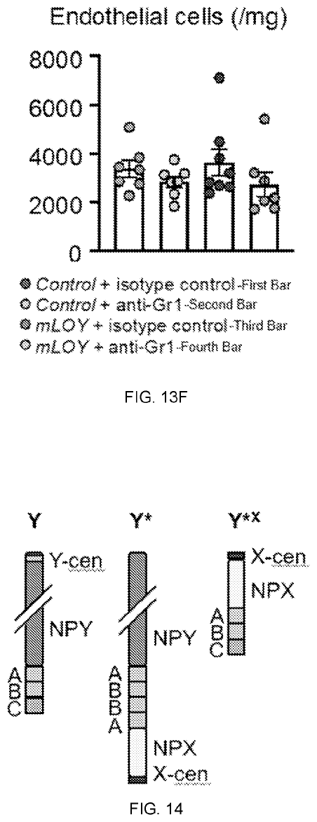

Consistent with the transcriptome analyses, elevated myocardial TGFB1 protein could be detected by immunoblot analysis in the mLOY condition compared to control at 1-week post-TAC (FIG. 13A). Elevated TGFB signaling in the myocardium was also indicated by the increase in the phosphorylation of SMAD2, a transcriptional regulator that is a major downstream target of TGFB1 (FIG. 13B). To examine TGFB signaling at a cellular level, SMAD2 phosphorylation was assessed by fluorescence immunohistochemistry. Consistently, the total number of phosphorylated SMAD2 positive cells was significantly higher in mLOY mice compared to control mice at 1 week after the TAC operation, as was a quantitative analysis of phosphorylated SMAD2-positive fibroblasts identified by co-immunostaining with vimentin (FIG. 11D). Collectively, these results suggest that Y chromosome deficiency upregulates the TGFβ signaling network in macrophages, and thereby promotes fibroblast activation and cardiac tissue fibrosis.

### Example 4

### Tgfβ Neutralization Reverses the Cardiac Dysfunction Observed in mLOY Mice

To assess the necessity of TGFβ signaling in accelerated cardiac dysfunction caused by the experimental mLOY condition, anti-TGFβ monoclonal antibody or control IgG was administered to mLOY mice that had undergone TAC surgery. Sequential echocardiographic analysis revealed that treatment with anti-TGFβ monoclonal antibody reversed the accelerated cardiac dysfunction observed in mLOY mice (FIGS. 11E and 13C). The anti-TGFβ antibody also reversed the mLOY-mediated increases in serum BNP levels, heart weight, and lung congestion (FIGS. 13D and 13E). Consistent with the recognized functions of TGFβ 1 to promote fibroblast proliferation and their conversion to myofibroblasts that produce higher amounts and matricellular proteins, the neutralizing anti-TGFβ antibody suppressed the increase in fibroblast number and extracellular matrix deposition that was associated with the mLOY condition, but did not alter endothelial cell number (FIGS. 11F, 11G, and 13F). These results indicate that elevation of a TGFβ signaling network in Y chromosome-deficient macrophages could contribute to the accelerated cardiac dysfunction in the mLOY mice.

### Discussion of Examples 1-4

Tissue fibrosis is a hallmark of aging and is estimated to contribute to 45% of deaths in industrialized countries (Rockey et al., 2015). Myocardial fibrosis results from the activation of cardiac-resident fibroblast and is often associated with heart failure, a major cause of mortality and morbidity in the elderly. Myocardial fibrosis can be triggered by bone marrow-derived macrophages that acutely infiltrate the heart in response to various forms of cardiac injury or progressively replace the cardiac-resident, yolk sac-derived macrophages with age (Rhee & Lavine, 2020; Wang et al., 2020). Here, we provide evidence supporting a causal link between hematopoietic mLOY and age-dependent cardiac dysfunction and heart failure in males. We report that Y chromosome-deficient cardiac macrophages over-activate a TGFβ signaling network, leading to cardiac fibroblast proliferation and activation, excessive matrix production and diminished heart function. Whereas there is a high co-occurrence of mLOY with CHIP (Zink et al., 2017; Ljungstrom et al., 2022), the observation of elevated TGFB signaling in mLOY is unexpected when compared with findings of the proinflammatory mechanisms by which CHIP mechanistically contributes to cardiovascular disease (Fuster et al., 2017; Sano et al., 2018a; Sano et al., 2018b; Yura et al., 2021). It is increasingly recognized that chronic diseases are caused by a spectrum of inflammation- and fibrosis-driven events (Wijsenbeek & Cottin, 2020). However, the interrelationship between inflammation and fibrosis is not simply reciprocal in that chronic inflammation will promote fibrosis, whereas fibrosis can function in the resolution of inflammatory processes. Thus, the somatic mosaicism that develops in the hematopoietic system with age may give rise to a complex interplay of pro- and anti-inflammatory processes that can differentially impact disease development. Finally, our experimental studies also found that a neutralizing TGFB antibody could reverse the pathological cardiac phenotypes caused by mLOY.

### Example 5

### Identification of the Uty Gene Target on the Y Chromosome that Confers (at Least in Part) the Effect of mLOY on Pathology

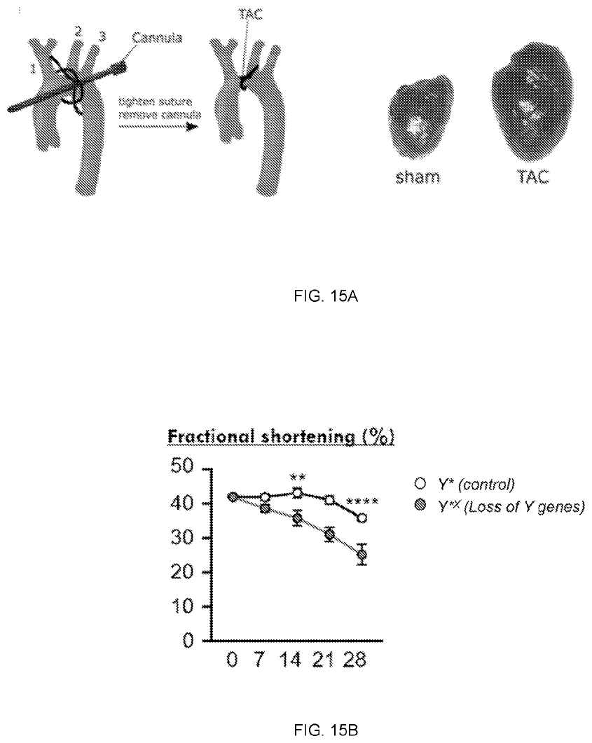

The model of mLOY disclosed herein employed CRISPR/Cas9 to ablate the Y chromosome in HSPC. See also Sano et al., 2022. To corroborate data in an independent model, we utilized a strain of mice that maintains a centromere, to ensure proper chromosome segregation and stability, but lacks the long and short arms of the Y chromosome (99% deletion of PAR region). This Y chromosome replacement variant is referred to as Y*^{X}. See FIG. 14. For our BMT experiments, the control is the Y* variant that contains the Y*^{X} centromere and the full complement of Y chromosome PAR region. This system has the representative non-limiting advantages of allowing corroboration our other findings with a system that avoids CRISPR/Cas9 methodology (that can theoretically be confounded by off-target editing); avoiding the potential issue of centromere loss that can lead to autosome mis-segregation, and providing a well-described and stable system that provides an alternative experimental methodology.

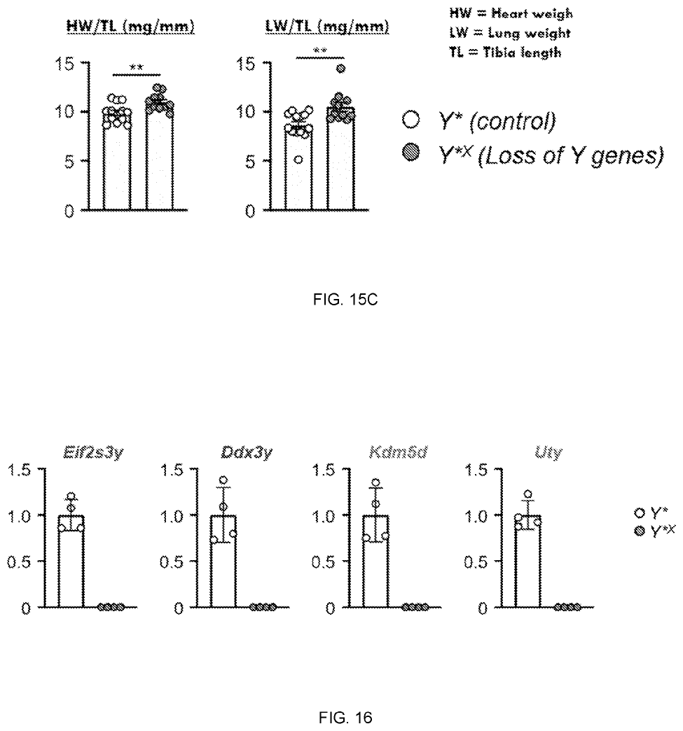

Referring to FIGS. 15A-15C, experiments that corroborate the findings with the CRISPR approach to LOY disclosed elsewhere herein (see also Sano et al. Science 2022). These experiments show that the Y*^{X} model of LOY leads to cardiac dysfunction and also suggests that the LOY effect in the transverse aortic constriction (TAC) model is due to loss of Y gene(s). FIG. 15A shows a pressure overload hypertrophy model showing Transverse Aortic Constriction (TAC). FIG. 15B is a graph showing fractional shortening (FS, %). FIG. 15C is a series of graphs suggesting that the LOY effect in the TAC model is due to loss of Y gene(s).

Referring to FIG. 16, a series of plots show four Y chromosome-encoded genes that are appreciably expressed in mouse leukocytes and are void in the LOY condition. These genes are Eif2s3y, Ddx3y, Kdm5d, and Uty.

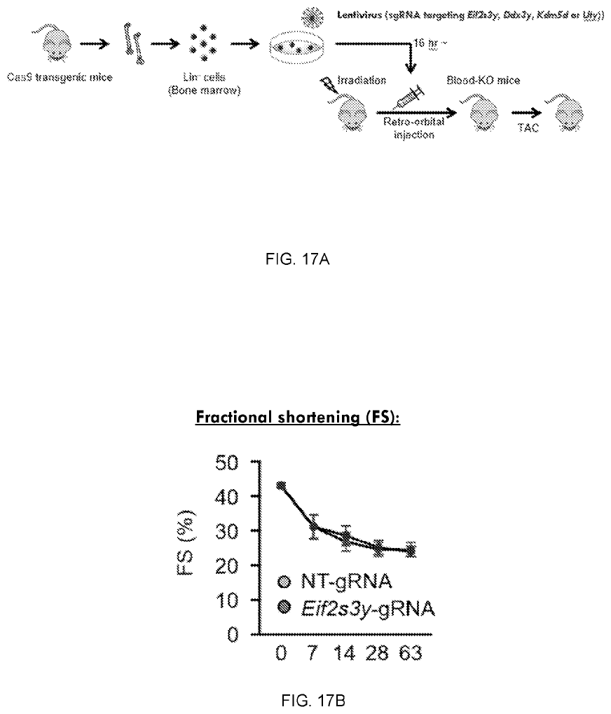

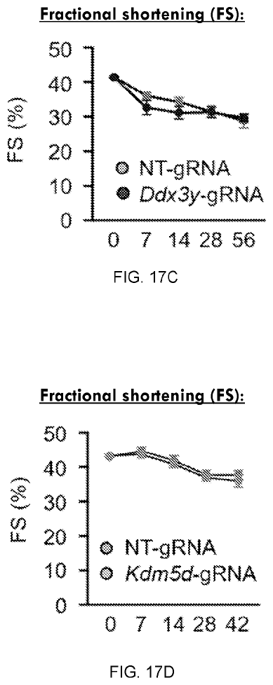

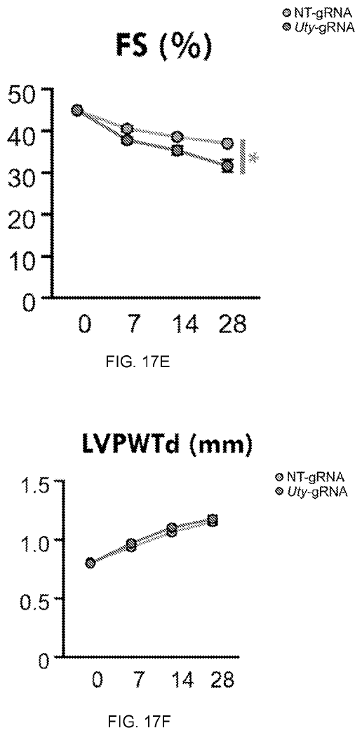

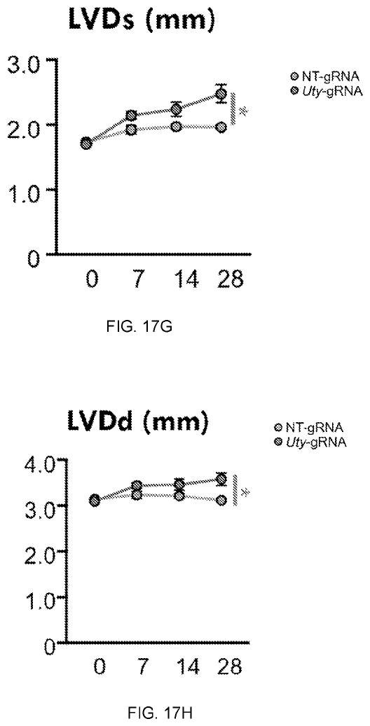

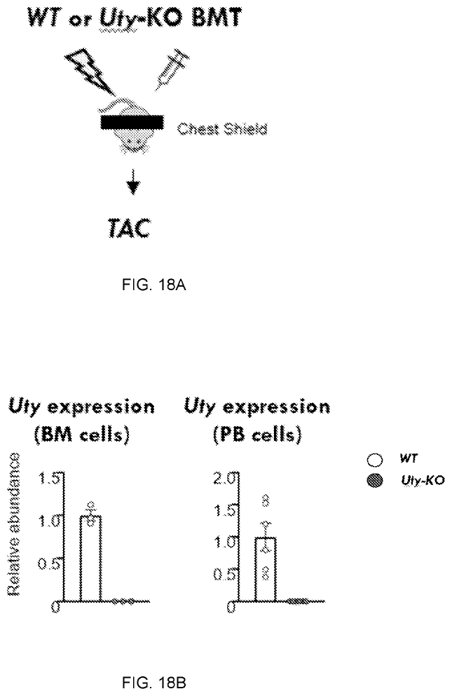

Referring to FIGS. 17A-17H, it was observed that CRISPR screening indicates only Uty loss affects cardiac function. An exemplary model TAC, a model of pressure overload cardiac hypertrophy, was employed. See FIG. 17A. FIGS. 17B-17D show fractional shortening associated with Eif2s3y, Ddx3y, and Kdm5d. Ablation of Eif2s3y, Ddx3y and Kdm5d showed no detectable effects on cardiac dysfunction in the TAC model. FIGS. 17E-17H show CRISP-mediated Uty ablation. CRISPR-mediated Uty ablation reveals greater pathological cardiac remolding in response to TAC.

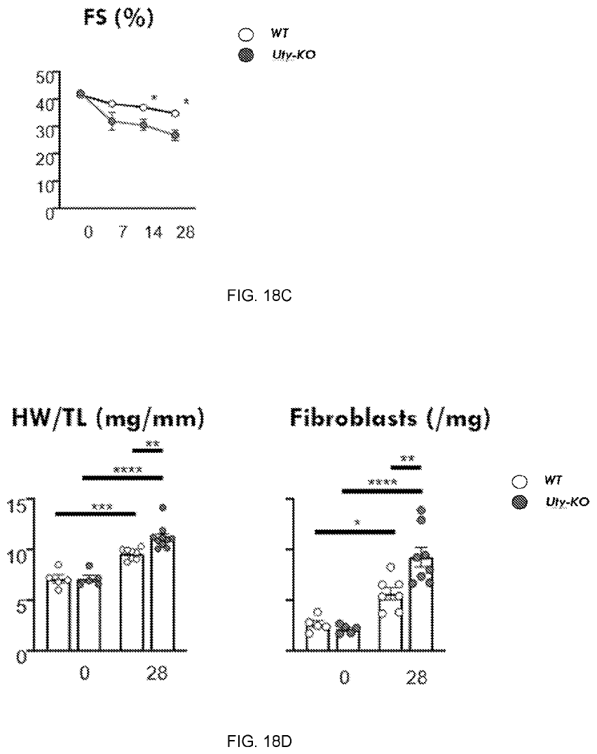

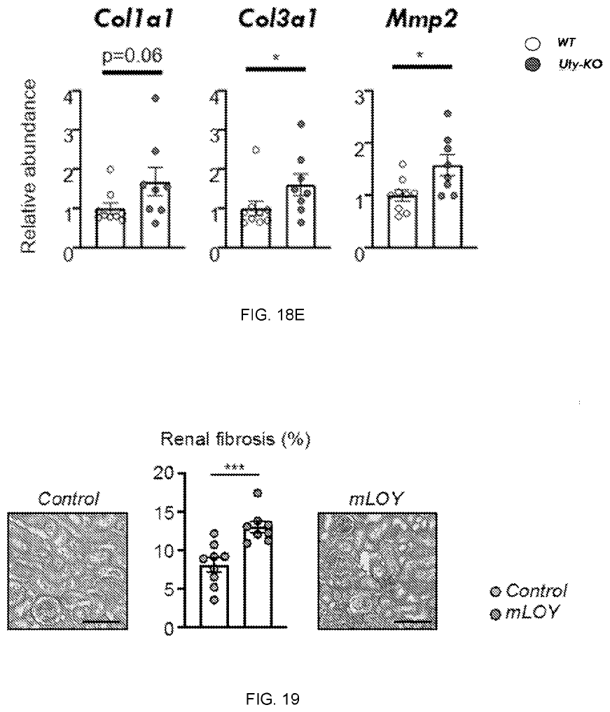

FIGS. 18A-18E show that bone marrow transplant (BMT) of Uty-deficient cells results in the same cardiac phenotype as the LOY mice, in the TAC model. An exemplary model of wild type (WT) or Uty-knockout (KO) bone marrow transplant mice employed in a TAC model is shown. See FIG. 18A. FIG. 18B shows Uty expression in bone marrow (BM) and peripheral blood (PB) cells, Y chromosome-encoded gene is void in the LOY condition. FIGS. 18C-18E are graphs show that Uty as a gene target on the Y chromosome confers, at least in part, the effect of mLOY on cardiac pathology. Here, FS=fractional shortening, HW=heart weight and TL=Tibia length. Collectively, these data identify Uty as a gene target on the Y chromosome that confers, at least in part, the effect of mLOY on cardiac pathology.

### Example 6

### Mloy/Uty-Deficiency Contributes to Kidney Dysfunction

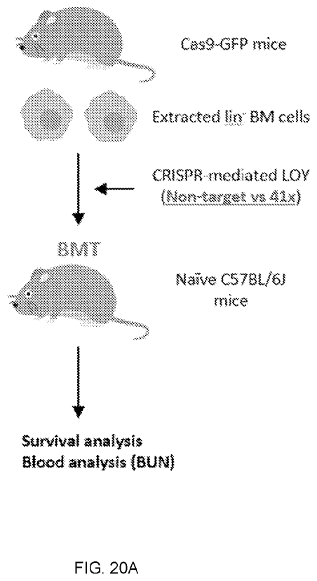

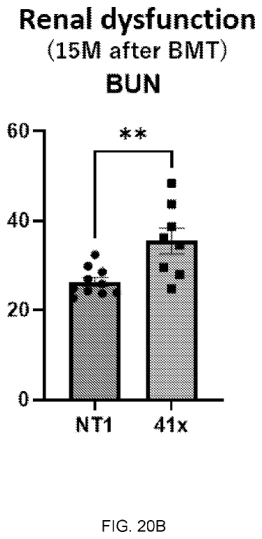

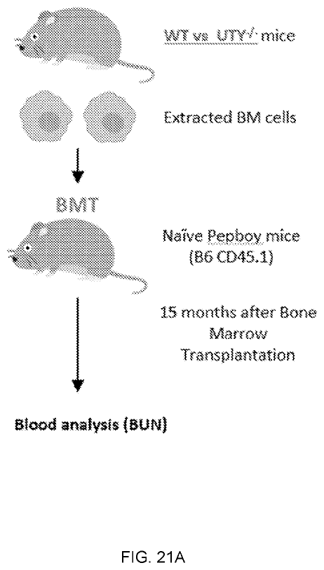

FIG. 19 shows renal fibrosis in aging mLOY mice. Quantitative analysis of fibrotic area in kidney sections, and representative images, at 60 weeks after bone marrow transplantation in mLOY and control mice (n=8-10 per group). Statistical analyses were performed using un-paired Student's t test. Scale bar: 0.1 mm. ***p<0.005. Also, mLOY is significantly associated with Chronic Kidney Disease (CKD)-related causes of death based on epidemiological data.

TABLE 3

| Registered causes of death | HR (95% CI) | Adj. p value |
|---|---|---|
| Heart failures | 1.65 (0.95-2.85) | 0.0745 |
| Peripheral artery diseases | 2.79 (1.30-5.97) | 0.0082 |
| Renal diseases | 1.97 (1.08-3.58) | 0.0265 |
| Renal failures | 2.15 (1.18-3.92) | 0.0121 |

mLOY is significantly associated with Chronic Kidney Disease (CKD)-related causes of death.

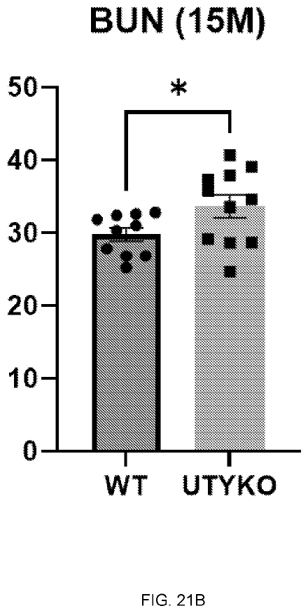

Referring to FIGS. 20A-20B, study of renal dysfunction in aging mLOY (CRISPR/Cas9) mice is shown, using an exemplary model of CRISPR-mediated LOY Referring to FIG. 20B, mLOY mice show increased Blood Urea Nitrogen (BUN) levels, a biomarker of kidney dysfunction. Referring to FIGS. 21A-21B, renal dysfunction in 15-month-old hematopoietic Uty-KO mice is evaluated in an exemplary model of Uty-KO mice. FIG. 21B shows that aged hematopoietic mosaic Uty^{-/-} mice display higher BUN levels.

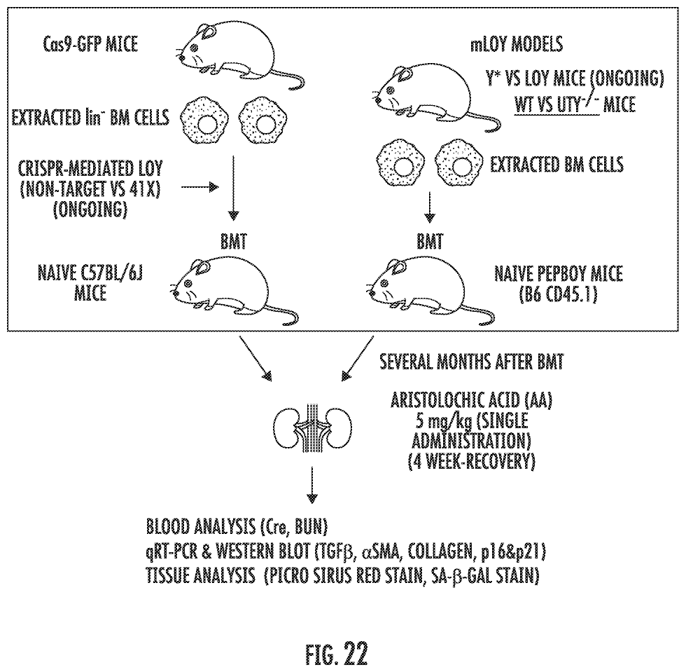

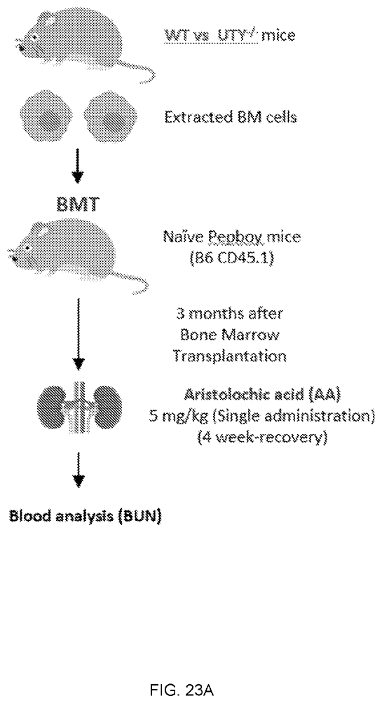

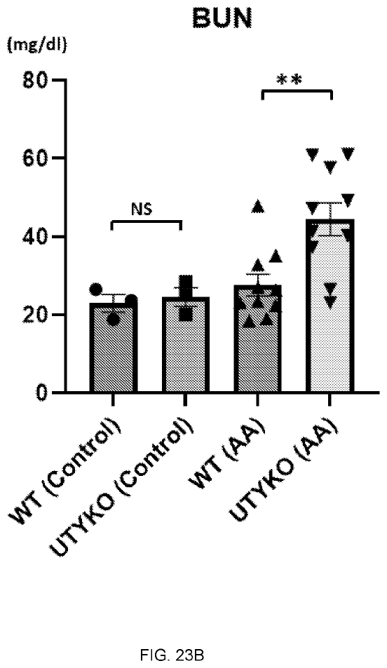

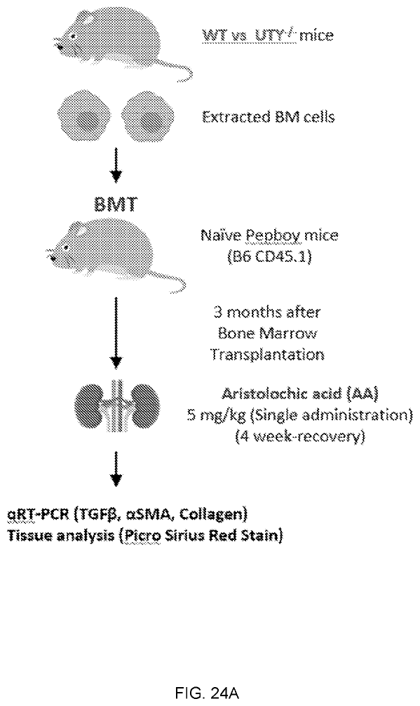

FIG. 22 schematically presents an aristolochic acid (AA)-induced chronic kidney disease (CKD) model, which is used for corroboration in a 2nd model of kidney dysfunction. Aristolochic acid (AA) 5 mg/kg is administered in a single administration with 4 week-recovery), followed by blood analysis (Cre, BUN) qRT-PCR & Western blot (TGFβ, αSMA, Collagen, p16&p21), and tissue analysis (Picro Sirius Red Stain, SA-3-gal stain).

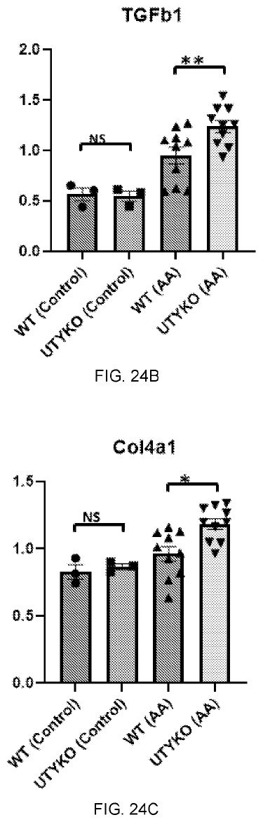

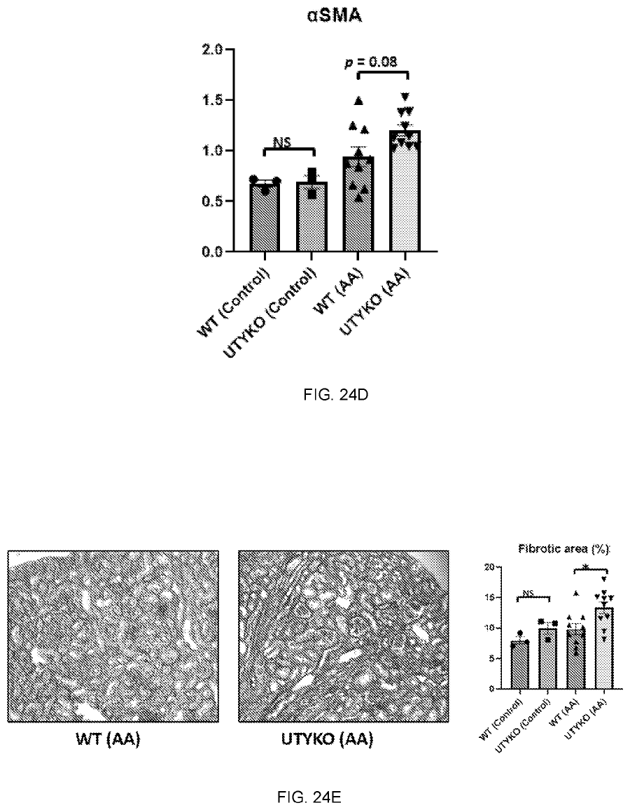

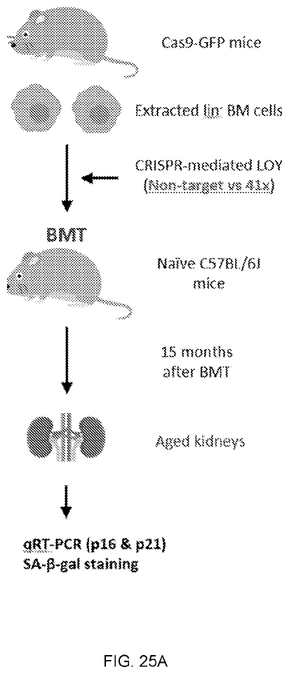

Referring to FIGS. 23A-23B and FIGS. 24A-24E, in the AA CKD model, elevated renal fibrosis with hematopoietic Uty-KO is observed. Hematopoietic Uty^{-/-} mice show more severe renal dysfunction (such by assessing Blood Urea Nitrogen; FIG. 23B). Referring to FIGS. 24B-24E, graphs and staining show hematopoietic Uty^{-/-} mice display more severe renal fibrosis.

### Example 7

### mLOY/Uty-Deficiency Promotes Cellular Senescence in Kidney and Senolytic Agents can Improve Pathology that Results from LOY/Uty-Deficiency

mLOY promotes macrophage polarization to a state that is “M2-like”; i.e. pro-fibrotic, quiescent and anti-inflammatory (Sano et al., 2022). Aging is characterized by macrophage polarization to an “M2-like” profibrotic phenotype that leads to macrophage expression of Tgfb1, fibroblast activation and tissue fibrosis. (Mahbub et al.; Meschiari et al.; Cui et al.; Stahl et al.; Tominaga & Suzuki.; Kale et al.). Thus, it was hypothesized that mLOY/Uty in blood cells can result in senescent cell accumulation, contributing to tissue dysfunction and biological aging.

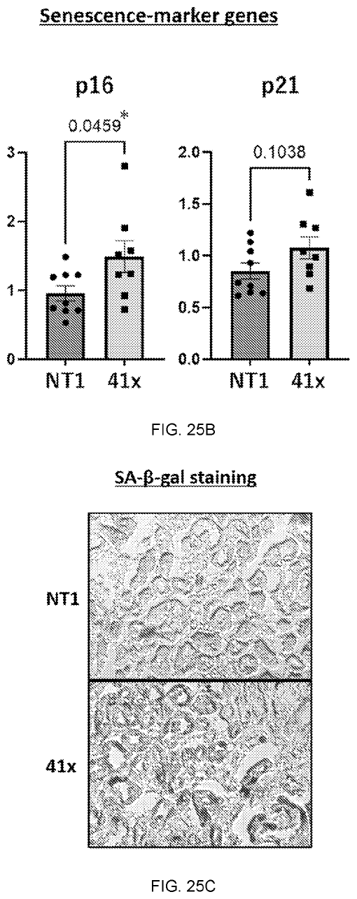

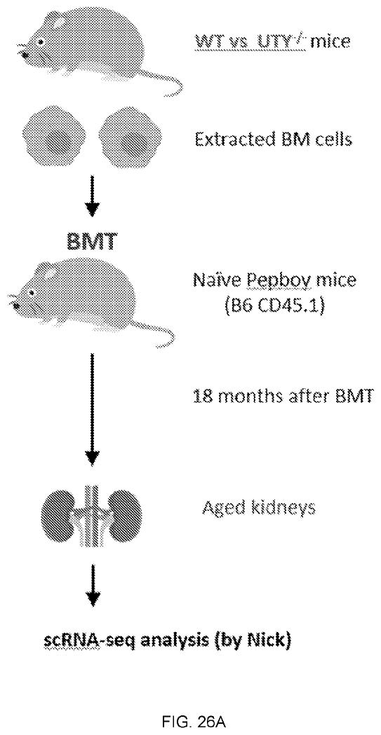

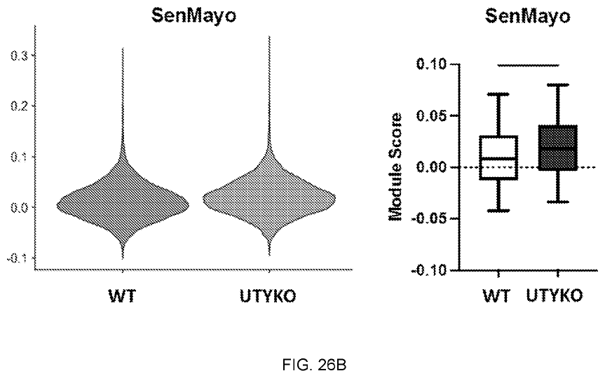

Referring to FIGS. 25A-25C, kidneys of aging mLOY mice (CRISPR/Cas9) display elevated markers of cellular senescence in an exemplary model of CRISPR-mediated LOY. Kidneys of mLOY mice show increased p16 & p21 (senescence-marker genes) expression and SA-3-gal staining shows positive in mLOY mice. Thus, it was further explored whether elevated levels of cellular senescence contribute to the pathological actions of MLOY, such as mortality, kidney disease, and other conditions, including but not limited to those disclosed elsewhere herein.

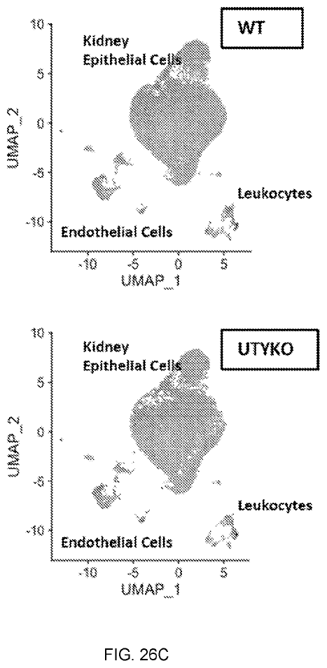

FIGS. 26A-26C show assessments of senescent kidneys in aging hematopoietic Uty-KO mice, using an exemplary model of wild type (WT) or Uty-knockout (KO) aged kidneys. As shown in FIGS. 26B-26C, kidneys in hematopoietic Uty^{-/-} mice display increased expression of the SenMayo diagnostic gene set. SenMayo is a gene set including 119 genes related to cellular senescence, Saul et al., 2022.

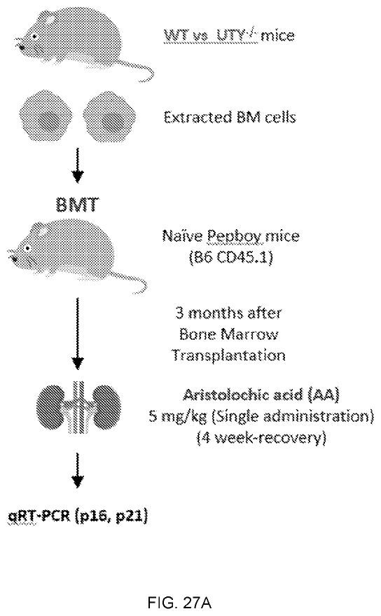

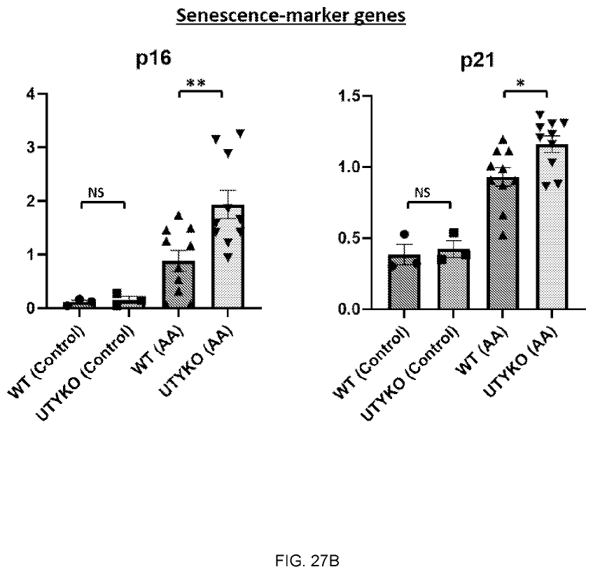

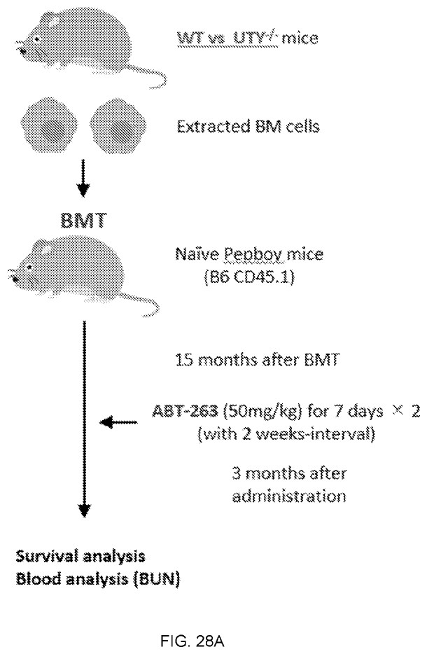

FIGS. 27A-27B show assessments of the AA CKD model with hematopoietic Uty-KO (Analysis of cellular senescence markers). Hematopoietic Uty^{-/-} mice show a higher expression of senescence-marker genes in kidneys in the AA CKD model.

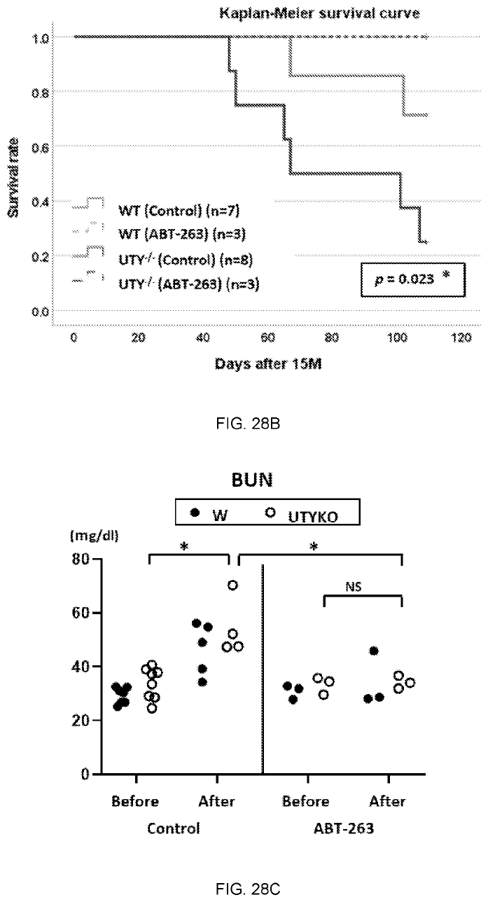

FIGS. 28A-28C show the effect of the senolytic ABT-263 on lifespan and kidney function in aging hematopoietic Uty-KO mice, using an exemplary model of ABT-263 mice. ABT-263 promotes survival and suppresses the progression of renal dysfunction in aging mice.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Adikusuma et al. (2017) Targeted deletion of an entire chromosome using CRISPR/Cas9. Mol Ther 25:1736-1738.
Aghajanian et al. (2019) Targeting cardiac fibrosis with engineered T cells. Nature 573:430-433.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Bird et al. (1988) Single-chain antigen-binding proteins. Science 242:423-426
Cui et al. Skewed macrophage polarization in aging skeletal muscle. Aging Cell. 2019.
Daley et al. (2008) Use of Ly6G-specific monoclonal antibody to deplete neutrophils in mice. J Leukoc Biol 83:64-70.
Danielsson et al. (2020) Longitudinal changes in the frequency of mosaic chromosome Y loss in peripheral blood cells of aging men varies profoundly between individuals. Eur J Hum Genet 28:349-357.
Devereux et al. (1984) A comprehensive set of sequence analysis programs for the VAX. Nucl Acids Res 12:387.
Dorsheimer et al. (2019) Association of Mutations Contributing to Clonal Hematopoiesis With Prognosis in Chronic Ischemic Heart Failure. JAMA Cardiol 4:25-33.
Dumanski et al. (2015) Mutagenesis. Smoking is associated with mosaic loss of chromosome Y. Science 347:81-83.
Dumanski et al. (2016) Mosaic loss of chromosome Y in blood is associated with Alzheimer Disease. Am J Hum Genet 98:1208-1219.
Dumanski et al. (2021) Immune cells lacking Y chromosome show dysregulation of autosomal gene expression. Cell Mol Life Sci 78:4019-4033.
Forsberg et al. (2014) Mosaic loss of chromosome Y in peripheral blood is associated with shorter survival and higher risk of cancer. Nat Genet 46:624-628.
Forsberg et al. (2019) Mosaic loss of chromosome Y in leukocytes matters. Nat Genet 51:4-7.
Fujiwara et al. (2020) Effects of pirfenidone targeting the tumor microenvironment and tumor-stroma interaction as a novel treatment for non-small cell lung cancer. Sci Rep 10:10900.
Fuster et al. (2017) Clonal hematopoiesis associated with TET2 deficiency accelerates atherosclerosis development in mice. Science 355:842-847.
Genaro (ed.) (1985) Remington's Pharmaceutical Sciences, Mack Publishing Co., Easton, Pennsylvania, United States of America.
Gennaro (ed.) (1990) Remington's Pharmaceutical Sciences, 18th ed., Mack Pub. Co., Easton, Pennsylvania, United States of America,
Gennaro (ed.) (2003) Remington: The Science and Practice of Pharmacy, 20th edition Lippincott, Williams & Wilkins, Philadelphia, Pennsylvania, United States of America.
Gross & Mienhofer (eds.) (1981) The Peptides, Vol. 3, Academic Press, New York, New York, United States of America. pages 3-88.
Haitjema et al. (2017) Loss of Y chromosome in blood is associated with major cardiovascular events during follow-up in men after carotid endarterectomy. Circ Cardiovasc Genet 10:e001544.
Harlow & Lane, 1988, Antibodies, A Laboratory Manual, Cold Spring Harbor Publications, Cold Spring Harbor, New York, United States of America.
Huston et al. (1988) Protein engineering of antibody binding sites: recovery of specific activity in an anti-digoxin single-chain Fv analogue produced in Escherichia coli. Proc Natl Acad Sci USA 85:5879.
Jobling & Tyler-Smith (2017) Human Y-chromosome variation in the genome-sequencing era. Nat Rev Genet 18:485-497.
Jones et al (1986) Replacing the complementarity-determining regions in a human antibody with those from a mouse. Nature 321:522.
Kale et al. Role of immune cells in the removal of deleterious senescent cells. Immun Ageing. 2020
Karlin & Altschul (1990) Methods for assessing the statistical significance of molecular sequence features by using general scoring schemes. Proc Natl Acad Sci USA 87(6):2264-2268.
Karlin & Altschul (1993) Applications and statistics for multiple high-scoring segments in molecular sequences. Proc Natl Acad Sci USA 90(12):5873-5877.
King, Jr. et al. (2014) A phase 3 trial of pirfenidone in patients with idiopathic pulmonary fibrosis. N Engl J Med 370:2083-2092.
Lewis et al. (2021) Pirfenidone in heart failure with preserved ejection fraction: a randomized phase 2 trial. Nat Med 27:1477-1482.
Ljungstrom et al. (2022) Loss of Y and clonal hematopoiesis in blood-two sides of the same coin? Leukemia 36:889-891.
Loftfield et al. (2018) Predictors of mosaic chromosome Y loss and associations with mortality in the UK Biobank. Sci Rep 8:12316.
Mahbub et al. Advanced age impairs macrophage polarization. J Interferon Cytokine Res. 2012.
Meschiari et al. The impact of aging on cardiac extracellular matrix. Geroscience 2017.
Patel et al. (1999) Impact of chimeric immune receptor extracellular protein domains on T cell function. Gene Therapy 6:412-419.
Pegram et al. (2012) Tumor-targeted T cells modified to secrete IL-12 eradicate systemic tumors without need for prior conditioning. Blood 119:4133-4141.
Rhee & Lavine (2020) New Approaches to Target Inflammation in Heart Failure: Harnessing Insights from Studies of Immune Cell Diversity. Annu Rev Physiol 82:1-20.
Riaz et al. (2021) A polygenic risk score predicts mosaic loss of chromosome Y in circulating blood cells. Cell Biosci 11:205.
Riechmann et al. (1988) Reshaping human antibodies for therapy. Nature 332(6162):323-327.
Rockey et al. (2015) Fibrosis—a common pathway to organ injury and failure. N Engl J Med 372:1138-1149.
Sano et al. (2018a) CRISPR-mediated gene editing to assess the roles of Tet2 and Dnmt3a in clonal hematopoiesis and cardiovascular disease. Circ Res 123:335-341.
Sano et al. (2018b) Tet2-mediated clonal hematopoiesis accelerates heart failure through a mechanism involving the IL-1beta/NLRP3 inflammasome. J Am Coll Cardiol 71:875-886.
Sano et al. (2019) Lentiviral CRISPR/Cas9-mediated genome editing for the study of hematopoietic cells in disease models. J Vis Exp 152:10.3791/59977.
Sano et al. (2022) Hematopoietic loss of Y chromosome leads to cardiac fibrosis and heart failure mortality. Science 377:292-297.
Saul et al. (2022) A new gene set identifies senescent cells and predicts senescence-associated pathways across tissues. Nat Commun 13(1):4827.
Stahl et al. Macrophages in the Aging Liver and Age-Related Liver Disease. Front Immunol. 2018.
Stephan et al. (2007) T cell-encoded CD80 and 4-1BBL induce auto- and transcostimulation, resulting in potent tumor rejection. Nat Med 13:1440-1449.
Thompson et al. (2019) Genetic predisposition to mosaic Y chromosome loss in blood. Nature 575:652-657.
Tominaga & Suzuki. TGF-β Signaling in Cellular Senescence and Aging-Related Pathology. Int J Mol Sci. 2019. Pro-inflammatory (M1-like) macrophages are critical for the clearance of senescent cells.
U.S. Patent Application Publication Nos. 2003/0017534, 2018/0298087, 2018/0312588, 2018/0346564, 2019/0151448,
U.S. Pat. Nos. 4,816,567; 5,482,856; 6,479,284; 6,677,436; 7,060,808; 7,906,625; 8,398,980; 8,436,150; 8,796,439; 8,802,374; 10,253,111.
Wang et al. (2020) Tet2-mediated clonal hematopoiesis in nonconditioned mice accelerates age-associated cardiac dysfunction. JCI Insight 5:e135204.
Wijsenbeek & Cottin (2020) Spectrum of fibrotic lung diseases. N Engl J Med 383:958-968.
Winter & Milstein (1991) Man-made antibodies. Nature 349(6307):293-299.
Yu et al. (2021) Supplemental association of clonal hematopoiesis with incident heart failure. J Am Coll Cardiol 78:42-52.
Yura et al. (2021) The cancer therapy-related clonal hematopoiesis driver gene Ppm1d promotes inflammation and non-ischemic heart failure in mice. Circ Res 129:684-698.
Zink et al. (2017) Clonal hematopoiesis, with and without candidate driver mutations, is common in the elderly. Blood 130:742-752.

TABLE 4
sgRNA Sequences

| NAME | Sequence* |
|---|---|
| LOY_gRNA1_fwd | CACCGGAGTTAATATAAAAAACA |
| | (SEQ ID NO: 1) |
| LOY_gRNA1_rv | AAACTGTTTTTTATATTAACTCC |
| | (SEQ ID NO: 2) |
| LOY_gRNA2_fwd | CACCGAAGAATTACAATGAAAAATA |
| | (SEQ ID NO: 3) |
| LOY_gRNA2_rv | AAACTATTTTTCATTGTAATTCTTC |
| | (SEQ ID NO: 4) |
| Control_(Neo)_gRNA1_ | CACCGGCAGCGCGGCTATCGTGGC |
| fwd | (SEQ ID NO: 5) |
| Control_(Neo)_gRNA1_ | AAACGCCACGATAGCCGCGCTGCC |
| fwd | (SEQ ID NO: 6) |
| hU6_fwd | GAGGGCCTATTTCCCATGATT |
| | (SEQ ID NO: 7) |

*SEQ ID NOs: 1-6 were employed for ex vivo genome editing, whereas SEQ ID NO: 7 was employed for Sanger sequencing.

TABLE 5
Antibodies used for flow cytometric analysis.

| | | | | |
|---|---|---|---|---|
| ANTIBODIES | FLUOROPHORE | CLONE | SOURCE | IDENTIFIER |

PERIPHERAL BLOOD

| | | | | |
|---|---|---|---|---|
| Anti-CD45.2 | eFluor450 | 104 | Thermo Fisher | Cat# 48-0454-80; |
| | | | | RRID: AB_11039533 |
| Anti-CD45.1 | PE-Cy7 | A20 | Invitrogen | Cat# 25-0453-82 |
| Anti-CD115 | PE-Cy7 | AFS98 | Invitrogen | Cat# 25-1152-82 |
| Anti-CD115 | PE | AFS98 | Thermo Fisher | Cat# 12-1152-81; |
| | | | | RRID: AB_465807 |
| Anti-Gr-1 | BV605 | RB6-8C5 | BioLegend | Cat# 108439 |
| Anti-Ly6G | PerCP-Cy5.5 | 1A8 | BD Biosciences | Cat# 560602 |
| Anti-Ly6C | APC | AL-21 | BD Biosciences | Cat# 560595 |
| Anti-CD45R | APC-Cy7 | RA3-6B2 | BD Biosciences | Cat# 552094 |
| Anti-CD3e | PE-eFluor610 | 145-2C11 | Thermo Fisher | Cat# 61-0031-80; |
| | | | | RRID: AB_2574513 |
| Anti-CD3e | BV711 | 145-2C11 | Biolegend | Cat# 100349 |
| Anti-CD4 | FITC | RM4-5 | Thermo Fisher | Cat# 11-0042-81; |
| | | | | RRID: AB_464895 |
| Anti-CD8a | BV510 | 53-6.7 | BioLegend | Cat# 100752; |
| | | | | RRID: AB_2563057 |

HEART

| | | | | |
|---|---|---|---|---|
| Anti-CD45 | PerCP-Cy5.5 | 30-F11 | BioLegend | Cat# 103131; |
| | | | | RRID: AB_{—} |
| Anti-CD45.1 | PE | A20 | BioLegend | Cat# 110707; |
| | | | | RRID: AB_{—} |
| Anti-CD45.2 | PerCP-Cy5.5 | 104 | BioLegend | Cat# 109828; |
| Anti-Ly6G | PE | 1A8 | BioLegend | Cat# 127602; |
| | | | | RRID: AB_1089180 |
| Anti-Ly6C | FITC | HK1.4 | BioLegend | Cat# 128006; |
| | | | | RRID: AB_1186135 |
| Anti-CD64 (FcgRI) | APC | X54-5/7.1 | BioLegend | Cat# 139306 |
| Anti-CD192 (CCR2) | BV421 | SA203G11 | BioLegend | Cat# 150605 |
| Anti-I-A/I-E | APC-Cy7 | M5/114.15.2 | BioLegend | Cat# 107627 |
| Anti-CD31 | BV421 | 390 | BioLegend | Cat# 102423 |
| Anti-Feeder Cell | APC | mEF-SK4 | Miltenyi Biotec | Cat# 130-120-802 |

TABLE 6
Primer sequences for PCR analyses.

| | Gene | | | |
|---|---|---|---|---|
| SEQ ID Number | Name | Species | Direction | Primer Sequence |
| SEQ ID NO. 8 | Nppa | Mus Musculus | Forward | 5′-AAGAACCTGCTAGACCACCTG-3′ |
| SEQ ID NO. 9 | Nppa | Mus Musculus | Reverse | 5′-TGCTTCCTCAGTCTGCTCAC-3′ |
| SEQ ID NO. 10 | aMHC | Mus Musculus | Forward | 5′-GCAGCAGCCCAGTACCTC-3′ |
| SEQ ID NO. 11 | aMHC | Mus Musculus | Reverse | 5′-GTCATCAGGCACGAAGCA-3′ |
| SEQ ID NO. 12 | bMHC | Mus Musculus | Forward | 5′-CCTCACATCTTCTCCATCTCTG-3′ |
| SEQ ID NO. 13 | bMHC | Mus Musculus | Reverse | 5′-TTGGATGACCCTCTTAGTGTTG-3′ |
| SEQ ID NO. 14 | 36b4 | Mus Musculus | Forward | 5′-GCTCCAAGCAGATGCAGCA-3′ |
| SEQ ID NO. 15 | 36b4 | Mus Musculus | Reverse | 5′-CCGGATGTGAGGCAGCAG-3′ |

It will be understood that various details of the presently disclosed subject matter can be changed without departing from the scope of the presently disclosed subject matter. Furthermore, the foregoing description is for the purpose of illustration only, and not for the purpose of limitation.

1-4. (canceled)

5. A method for preventing and/or treating a disease, disorder, and/or condition associated with mosaic loss of chromosome Y in blood (mLOY) in a subject in need thereof, the method comprising administering to the subject an inhibitor of TGFβ signaling via a route and in an amount sufficient to inhibit TGFβ signaling in the subject and/or administering an effective amount of a senolytic agent to the subject, whereby a disease, disorder, and/or condition associated with mosaic loss of chromosome Y in blood (mLOY) in the subject is treated.

6. The method of claim 5, wherein the inhibitor of TGFβ signaling is an anti-TGFβ antibody or a fragment thereof that binds to a TGFβ polypeptide to inhibit TGFβ signaling in the subject; a nucleic acid molecule that binds to and inhibits expression of a TGFβ gene product in the subject; a small molecule inhibitor of TGFβ signaling, optionally pirfenidone; or any combination thereof.

7. The method of claim 5, wherein the senolytic agent is selected from the group consisting of FOXO4-related peptide, a BCL-2 inhibitor, a Src inhibitor, a USP7 inhibitor, N-(2-chloro-6-methylphenyl)-2-[[6-[4-(2-hydroxyethyl)-1-piperazinyl]-2-methyl-4-pyrimidinyl]amino]-5-thiazole carboxamide monohydrate and 2-(3,4-dihydroxyphenyl)-3,5,7-trihydroxychromen-4-one, Fisetin (3,3′,4′,7-tetrahydroxyflavone), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-trifluoromethanesulfonyl)benzene-1-sulfonyl)benzamide, azithromycin and roxithromycin, a senescence-specific killing compound 1, (2R,3S,4S,5R,6S)-2-(acetoxymethyl)-6-(4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)-2-nitrophenoxy)tetrahydro-2H-pyran-3,4,5-triyl triacetate, a BIRC5 inhibitor, a glutaminase-1 (GLS1) inhibitor, an anti-Glycoprotein Nmb (GPNMB) vaccine, a cardiac glycoside, 25-hydroxycholesterol (25HC), (2R,3R,4S)-2-(3,4-dihydroxyphenyl)-4-[(2R,3R)-2-(3,4-dihydroxyphenyl)-3,5,7-trihydroxy-3,4-dihydro-2H-chromen-8-yl]-8-[(2R,3R,4R)-2-(3,4-dihydroxyphenyl)-3,5,7-trihydroxy-3,4-dihydro-2H-chromen-4-yl]-3,4-dihydro-2H-chromene-3,5,7-triol, (3E,5E)-3,5-bis[(2-fluorophenyl)methylidene]piperidin-4-one, a heat shock protein 90 (HSP90) inhibitor, and any combination thereof.

8. The method of claim 5, wherein the disease, disorder, and/or condition associated with mLOY in the subject is a reduced lifespan, age-associated cardiomyopathy, reduced cardiac function, heart failure, increased fibrosis, optionally increased fibrosis of the myocardium, lung, and/or kidney; idiopathic pulmonary fibrosis (IPF); elevated left ventricular filling pressure (E/e′) indicative of diastolic dysfunction; and/or reduced cognitive function in the subject.

9. The method of claim 5, further comprising administering an anti-fibrotic therapy to the subject.

10. The method of claim 9, wherein the anti-fibrotic therapy comprises administering to the subject an effective amount of a small molecule anti-fibrotic, an anti-fibroblast antibody or a fragment thereof that binds to a polypeptide expressed by a fibroblast; a nucleic acid molecule that binds to and inhibits expression of a gene product expressed by a fibroblast in the subject; and a small molecule anti-fibrotic, or any combination thereof.

11. The method of claim 10, wherein the small molecule anti-fibrotic is a withanolide compound, a fused ring derivative of 2-{[(2E)-3-(3,4-dimethoxyphenyl)prop-2-enoyl]amino}benzoic acid, tranilast (n-[3,4-dimethoxycinnamoyl]anthranilic acid), pirfenidone, nintedanib, metabolites thereof, precursors thereof, or any combinations thereof.

12. The method of claim 9, wherein the anti-fibrotic therapy comprises an anti-fibroblast CAR-T cell therapy, and further wherein the anti-fibroblast CAR-T cell therapy employs CAR-T cells that are directed to fibroblast activation protein (FAP).

13-14. (canceled)

15. A method for preventing and/or treating a disease, disorder, and/or condition associated with mosaic loss of chromosome Y in blood (mLOY) in a subject in need thereof, the method comprising: determining if the subject has mosaic loss of chromosome Y in blood (mLOY), wherein the presence of mLOY in the subject is indicative of the subject being at risk for reduced lifespan, age-associated cardiomyopathy, reduced cardiac function, heart failure, increased fibrosis of the myocardium, lung, and/or kidney; idiopathic pulmonary fibrosis (IPF); elevated left ventricular filling pressure (E/e′) indicative of diastolic dysfunction; and/or reduced cognitive function; and administering to the subject an inhibitor of TGFβ signaling via a route and in an amount sufficient to inhibit TGFβ signaling in the subject and/or administering an effective amount of a senolytic agent to the subject, whereby a disease, disorder, and/or condition associated with mosaic loss of chromosome Y in blood (mLOY) in the subject is treated.

16. The method of claim 15, wherein the determining comprises assaying gene expression in macrophages isolated from the subject, optionally from the subject's blood or heart, for mLOY or comprises detecting a presence or absence of a Uty gene in sample from the subject.

17. The method of claim 15, wherein the determining employs RT-PCR analysis of RNA isolated from a cell isolated from the subject, optionally wherein the cell is a macrophage

18. The method of claim 16, wherein the gene expression assayed comprises TGFβ1 gene expression or Uty gene expression.

19. The method of claim 15, wherein the inhibitor of TGFβ signaling is an anti-TGFβ antibody or a fragment thereof that binds to a TGFβ polypeptide to inhibit TGFβ signaling in the subject; a nucleic acid molecule that binds to and inhibits expression of a TGFβ gene product in the subject; a small molecule inhibitor of TGFβ signaling, optionally pirfenidone; or any combination thereof.

20. Then method of claim 15, wherein the senolytic agent is selected from the group consisting of FOXO4-related peptide, a BCL-2 inhibitor, a Src inhibitor, a USP7 inhibitor, N-(2-chloro-6-methylphenyl)-2-[[6-[4-(2-hydroxyethyl)-1-piperazinyl]-2-methyl-4-pyrimidinyl]amino]-5-thiazole carboxamide monohydrate and 2-(3,4-dihydroxyphenyl)-3,5,7-trihydroxychromen-4-one, Fisetin (3,3′,4′,7-tetrahydroxyflavone), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-trifluoromethanesulfonyl)benzene-1-sulfonyl)benzamide, azithromycin and roxithromycin, a senescence-specific killing compound 1, (2R,3S,4S,5R,6S)-2-(acetoxymethyl)-6-(4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)-2-nitrophenoxy)tetrahydro-2H-pyran-3,4,5-triyl triacetate, a BIRC5 inhibitor, a glutaminase-1 (GLS1) inhibitor, an anti-Glycoprotein Nmb (GPNMB) vaccine, a cardiac glycoside, 25-hydroxycholesterol (25HC), (2R,3R,4S)-2-(3,4-dihydroxyphenyl)-4-[(2R,3R)-2-(3,4-dihydroxyphenyl)-3,5,7-trihydroxy-3,4-dihydro-2H-chromen-8-yl]-8-[(2R,3R,4R)-2-(3,4-dihydroxyphenyl)-3,5,7-trihydroxy-3,4-dihydro-2H-chromen-4-yl]-3,4-dihydro-2H-chromene-3,5,7-triol, (3E,5E)-3,5-bis[(2-fluorophenyl)methylidene]piperidin-4-one, a heat shock protein 90 (HSP90) inhibitor, and any combination thereof.

21. The method of claim 15, wherein the disease, disorder, and/or condition associated with mLOY in the subject is a reduced lifespan, age-associated cardiomyopathy, reduced cardiac function, heart failure, increased fibrosis, optionally increased fibrosis of the myocardium, lung, and/or kidney; idiopathic pulmonary fibrosis (IPF); elevated left ventricular filling pressure (E/e′) indicative of diastolic dysfunction; and/or reduced cognitive function in the subject.

22. The method of claim 15, further comprising administering an anti-fibrotic therapy to the subject.

23. The method of claim 22, wherein the anti-fibrotic therapy comprises administering to the subject an effective amount of a small molecule anti-fibrotic, an anti-fibroblast antibody or a fragment thereof that binds to a polypeptide expressed by a fibroblast; a nucleic acid molecule that binds to and inhibits expression of a gene product expressed by a fibroblast in the subject; and a small molecule anti-fibrotic, or any combination thereof.

24. The method of claim 23, wherein the small molecule anti-fibrotic is a withanolide compound, a fused ring derivative of 2-{[(2E)-3-(3,4-dimethoxyphenyl)prop-2-enoyl]amino}benzoic acid, tranilast (n-[3,4-dimethoxycinnamoyl]anthranilic acid), pirfenidone, nintedanib, metabolites thereof, precursors thereof, or any combinations thereof.

25. The method of claim 22, wherein the anti-fibrotic therapy comprises an anti-fibroblast CAR-T cell therapy, and further wherein the anti-fibroblast CAR-T cell therapy employs CAR-T cells that are directed to fibroblast activation protein (FAP).
